(19) United States
(12) Patent Application Publication
(10) Pub. No.: US 2021/0393773 A1
(43) Pub. Date: Dec. 23, 2021

(54) COMPOSITIONS COMPRISING BACTERIAL STRAINS

(71) Applicant: 4D Pharma Research Limited, Aberdeen (GB)

(72) Inventors: Emma Elizabeth Clare Hennessy, Aberdeen (GB); Suaad AHMED, Aberdeen (GB); Amy Beth HOLT, Aberdeen (GB); Philip COWIE, Aberdeen (GB); Anna ETTORRE, Aberdeen (GB); Samantha YUILLE, Aberdeen (GB); Imke Elisabeth MULDER, Aberdeen (GB)

(21) Appl. No.: 17/245,060

(22) Filed: Apr. 30, 2021

Related U.S. Application Data
(63) Continuation of application No. PCT/EP2019/080131, filed on Nov. 4, 2019.

(30) Foreign Application Priority Data
Nov. 2, 2018 (EP) 18204199.6
Dec. 12, 2018 (GB) 1820261.4

(51) Int. Cl. A61K 39/39; C12N 1/20

(52) U.S. Cl. CPC A61K 39/39; A61K 2039/52; C12N 1/205

(57) ABSTRACT

The invention provides compositions comprising bacterial strains for use as a vaccine adjuvant; for use in treating, preventing or delaying immunosenescence; or for use in enhancing a cell therapy, such as CAR-T. The invention also provides vaccine compositions comprising bacterial strains and one or more antigens.

### CROSS-REFERENCE

This application is a continuation of International Application No. PCT/EP2019/080131, filed Nov. 4, 2019, which claims the benefit of European Application No. 18204199.6, filed Nov. 2, 2018, and Great Britain Application No. 1820261.4, filed Dec. 12, 2018, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Apr. 16, 2021, is named 56708_752_301_SL.txt and is 44,713 bytes in size.

### TECHNICAL FIELD

This invention is in the field of compositions comprising bacterial strains from the mammalian digestive tract and the use of such compositions to induce a desirable immune response for the prevention or treatment of a variety of diseases, ranging from infections to cancer.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 500-1000 different phylotypes belonging essentially to two major bacterial divisions, the Bacteroidetes and the Firmicutes [2]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized and is exemplified in germfree animals which have an impaired immune system that is functionally reconstituted following the introduction of commensal bacteria [3-5].

Dramatic changes in microbiota composition have been documented in gastrointestinal disorders such as inflammatory bowel disease (IBD). For example, the levels of Clostridium cluster XIVa bacteria are reduced in IBD patients whilst numbers of E. coli are increased, suggesting a shift in the balance of symbionts and pathobionts within the gut [6-9]. Interestingly, this microbial dysbiosis is also associated with imbalances in T effector cell populations.

In recognition of the potential positive effect that certain bacterial strains may have on the animal gut, various strains have been proposed for use in the treatment of various diseases (see, for example, [10-13]). Also, certain strains, including mostly Lactobacillus and Bifidobacterium strains, have been proposed for use in treating various inflammatory and autoimmune diseases that are not directly linked to the intestines, for example through anti-inflammatory mechanisms (see [14] and [15] for reviews). Certain Streptococcus and Veillonella strains, and to a lesser extent, Enterococcus and Lactobaccillus strains have been suggested to have immunomodulatory effects, with varying effects on different cytokines in vitro. However, the relationship between different diseases and different bacterial strains, and the precise effects of particular bacterial strains on the gut and at a systemic level and on any particular types of diseases, are poorly characterised.

Recently, various Parabacteroides species have been investigated for their anti-inflammatory properties and therapeutic properties. For instance, Parabacteroides distasonis was demonstrated as having a broad anti-inflammatory effect in a number of disease models, such as severe asthma, rheumatoid arthritis and multiple sclerosis [16]. Parabacteroides distasonis has also been tested in an animal model of colorectal cancer [17]. Anti-inflammatory effects of Parabacteroides goldsteinii have also been observed [18].

There is a requirement in the art for new methods of treating diseases. There is also a requirement for the potential effects of gut bacteria to be characterised so that new therapeutic strategies using gut bacteria can be developed.

### SUMMARY OF THE INVENTION

The inventors have developed new compositions comprising a bacterial strain of the genus Parabacteroides that can be used as a vaccine adjuvant.

The invention therefore provides a composition comprising a bacterial strain of the genus Parabacteroides, for use as a vaccine adjuvant in a subject. Preferably, the invention provides a composition comprising a strain from the species Parabacteroides distasonis, Parabacteroides goldsteinii and/or Parabacteroides merdae. In preferred embodiments, the composition of the invention comprises a strain from the species Parabacteroides distasonis. In such embodiments, the strain may be that deposited under accession number 42382 at NCIMB, or a derivative or biotype thereof, for use as a vaccine adjuvant.

In further aspects, the invention provides a composition comprising a bacterial strain of the genus Parabacteroides, for use in enhancing a cell therapy, such as CAR-T. Preferably, the invention provides a composition comprising a strain from the species Parabacteroides distasonis, Parabacteroides goldsteinii and/or Parabacteroides merdae. In preferred embodiments, the composition of the invention comprises a strain from the species Parabacteroides distasonis. In such embodiments, the strain may be that deposited under accession number 42382 at NCIMB, or a derivative or biotype thereof, for use in enhancing a cell therapy, such as CAR-T.

In further aspects, the invention provides a composition comprising a bacterial strain of the genus Parabacteroides, for use in treating, preventing or delaying immunosenescence. Preferably, the invention provides a composition comprising a strain from the species Parabacteroides distasonis, Parabacteroides goldsteinii and/or Parabacteroides merdae. In preferred embodiments, the composition of the invention comprises a strain from the species Parabacteroides distasonis. In such embodiments, the strain may be that deposited under accession number 42382 at NCIMB, or a derivative or biotype thereof, for use in treating, preventing or delaying immunosenescence.

Most preferably, the bacteria used in the composition of the invention is the strain deposited under accession number 42382 at NCIMB.

In preferred embodiments, the composition of the invention is for use in increasing the secretion level and/or activity of monocyte chemoattractant protein-1 (MCP-1) and/or expansion of B-cells, as demonstrated in the examples. Preferably, the invention provides a composition comprising the strain deposited under accession number 42382 at NCIMB, or a derivative or biotype thereof, for use in increasing the expression level and/or activity of MCP-1 and/or expansion of B-cells when used as a vaccine adjuvant.

Strains closely related to the Parabacteroides strain tested in the examples are expected to be particularly effective at enhancing the efficacy of a vaccine. In preferred embodiments, the composition of the invention comprises a bacterial strain which has a 16s rRNA gene sequence that is at least 95%, 96%, 97%, 98%, 99%, 99.5% or 99.9% identical to SEQ ID NO:9 or wherein the bacterial strain has a 16s rRNA gene sequence represented by SEQ ID NO:9.

In certain embodiments, the composition of the invention is for oral administration. Oral administration of the bacterial strains of the invention may be effective for vaccine adjuvancy. Also, oral administration is convenient for patients and practitioners and allows delivery to and/or partial or total colonisation of the intestine.

In certain embodiments, the composition of the invention comprises one or more pharmaceutically acceptable excipients or carriers.

In certain embodiments, the composition of the invention comprises a bacterial strain that has been lyophilised. Lyophilisation is an effective and convenient technique for preparing stable compositions that allow delivery of bacteria.

In certain embodiments, the invention provides a food product comprising the composition as described above, for use in the medical uses defined above.

In certain embodiments, the invention provides a vaccine composition comprising a bacterial strain as described above and one or more antigens, such as pathogen antigens or tumour antigens. Pathogen antigens include viral antigens, such as viral surface proteins; bacterial antigens, such as protein and/or saccharide antigens; fungal antigens; and parasite antigens. Where the antigen is a bacterial antigen it will not usually be from a Parabacteroides strain.

In certain embodiments, vaccine compositions of the invention comprise one or more antigens from the following pathogens: influenza virus, HIV, hookworm, hepatitis B virus, herpes simplex virus, rabies, respiratory syncytial virus, cytomegalovirus, Staphylococcus aureus, chlamydia, SARS coronavirus, varicella zoster virus, Streptococcus pneumoniae, Neisseria meningitidis, Mycobacterium tuberculosis, Bacillus anthracis, Epstein Barr virus, or human papillomavirus. Preferably, vaccine compositions of the invention comprise one or more influenza virus antigens

In certain embodiments, vaccine compositions of the invention comprise one or more of neoantigens, glycoprotein antigens, lipoglycan antigens, archaea antigens, melanoma antigen E (MAGE), Carcinoembryonic antigen (CEA), MUC-1, HER2, sialyl-Tn (STn), human telomerase reverse transcriptase (hTERT), Wilms tumour gene (WT1), CA-125, prostate-specific antigen (PSA), oncoproteins, amyloid-beta, Tau, PCSK9 or habit forming substances such as nicotine, alcohol or opiates.

The invention further provides the vaccine compositions, as defined above, for use in medicine, in particular for use as defined above.

Additionally, the invention provides a method of enhancing the efficacy of a vaccine; enhancing a cell therapy, such as CAR-T; or treating, preventing or delaying immunosenescence; in a subject, comprising administering a composition comprising a bacterial strain of the genus Parabacteroides.

The invention also provides the following numbered embodiments:
1. A composition comprising a bacterial strain of the genus Parabacteroides, for use as a vaccine adjuvant.
2. A composition comprising a bacterial strain of the genus Parabacteroides, for use in treating, preventing or delaying immunosenescence.
3. A composition comprising a bacterial strain of the genus Parabacteroides, for use in enhancing a cell therapy, such as CAR-T.
4. The composition of any preceding embodiment, wherein the bacterial strain belongs to the species Parabacteroides distasonis, Parabacteroides goldsteinii or Parabacteroides merdae.
5. The composition of any preceding embodiment, wherein the bacterial strain has a 16s rRNA gene sequence that is at least 95%, 96%, 97%, 98%, 99%, 99.5% or 99.9% identical to SEQ ID NO: 1, 2, 3, 4, 5, 6, 7, 8, 9, 10, 11, 12, 13, 14, 15, 16, 17, 18, 19, 20, 21, 22 or 23, or wherein the bacterial strain has a 16s rRNA gene sequence represented by SEQ ID NO: 1, 2, 3, 4, 5, 6, 7, 8, 9, 10, 11, 12, 13, 14, 15, 16, 17, 18, 19, 20, 21, 22 or 23.
6. The composition of any preceding embodiment, wherein the bacterial strain has a 16s rRNA gene sequence that is at least 95%, 96%, 97%, 98%, 99%, 99.5% or 99.9% identical to SEQ ID NO:9 or wherein the bacterial strain has a 16s rRNA gene sequence represented by SEQ ID NO:9.
7. The composition of embodiment 6, wherein the bacterial strain is the strain deposited under accession number 42382 at NCIMB.
8. The composition of any of embodiments 1-5, wherein the bacterial strain has a 16s rRNA gene sequence that is at least 98%, 99%, 99.5% or 99.9% identical to SEQ ID NO: 9, 12, 13, 16, 17, or 19, or wherein the bacterial strain has a 16s rRNA gene sequence represented 9, 12, 13, 16, 17, or 19.
9. The composition of any of embodiments 1-5, wherein the bacterial strain has a 16s rRNA gene sequence that is at least 98%, 99%, 99.5% or 99.9% identical to SEQ ID NO: 10 or 11, or wherein the bacterial strain has a 16s rRNA gene sequence represented by SEQ ID NO: 10 or 11.
10. The composition of embodiment 9, wherein the bacterial strain is the strain deposited under either accession number DSMZ19448 or DSMZ29187.
11. The composition of any of embodiments, 1-5, wherein the bacterial strain has a 16s rRNA gene sequence that is at least 98%, 99%, 99.5% or 99.9% identical to SEQ ID NO: 18, or wherein the bacterial strain has a 16s rRNA gene sequence represented by SEQ ID NO: 18.
12. The composition of any of embodiments 1-3 or 5, wherein the bacterial strain has a 16s rRNA gene sequence that is at least 98%, 99%, 99.5% or 99.9% identical to SEQ ID NO: 15, or wherein the bacterial strain has a 16s rRNA gene sequence represented by SEQ ID NO: 15.
13. The composition of any of embodiments 1 or 4-12, wherein the composition comprises one or more pathogen or tumour antigens.
14. The composition of embodiment 13, wherein the one or more pathogen antigens are selected from viral antigens, such as viral surface proteins; bacterial antigens, such as protein and/or saccharide antigens; fungal antigens; and parasite antigens.
15. The composition of embodiment 14 wherein the one or pathogen antigens are from any of the following pathogens: influenza virus, HIV, hookworm, hepatitis B virus, herpes simplex virus, rabies, respiratory syncytial virus, cytomegalovirus, Staphylococcus aureus, chlamydia, SARS coronavirus, varicella zoster virus, Streptococcus pneumoniae, Neisseria meningitidis, Mycobacterium tuberculosis, Bacillus anthracis, Epstein Barr virus, or human papillomavirus.
16. The composition of embodiment 15, wherein the composition comprises one or more influenza virus antigens.
17. The composition of any of embodiments 1 or 4-13, wherein the composition comprises one or more antigens selected from neoantigens, glycoprotein antigens, lipoglycan antigens, archaea antigens, melanoma antigen E (MAGE), Carcinoembryonic antigen (CEA), MUC-1, HER2, sialyl-Tn (STn), human telomerase reverse transcriptase (hTERT), Wilms tumour gene (WT1), CA-125, prostate-specific antigen (PSA), oncoproteins, amyloid-beta, Tau, PCSK9 or habit forming substances such as alcohol or opiates.
18. The composition of any preceding embodiment, wherein the composition is for oral administration.
19. The composition of any preceding embodiment, wherein the composition comprises one or more pharmaceutically acceptable excipients or carriers.
20. The composition of any preceding embodiment, wherein the bacterial strain is lyophilised.
21. The composition of any preceding embodiment, wherein the bacterial strain expresses a heterologous antigen, such as a pathogen antigen or a tumour antigen.
22. The composition of any preceding embodiment, for use in increasing the expression level and/or activity of MCP-1, and/or the expansion of B-cells.
23. The composition of embodiment 22, wherein the B cells include CD19+CD3⁻ B cells.
24. The composition of any preceding embodiment, for use in inducing TNF-α cytokine production.
25. The composition of any preceding embodiment, for use in inducing IL-1β cytokine production.
26. The composition of any preceding embodiment, for use in inducing IL-2 cytokine production.
27. The composition of any preceding embodiment, for use in inducing GM-CSF cytokine production.
28. The composition of any preceding embodiment, for use in inducing IFN-γ cytokine production.
29. The composition of any preceding embodiment, for use in inducing IL-27 cytokine production.
30. The composition of any preceding embodiment, for use in inducing IP-10 cytokine production.
31. The composition of any preceding embodiment, for use in inducing RANTES cytokine production.
32. The composition of any preceding embodiment, for use in inducing MIP-1α cytokine production.
33. The composition of any preceding embodiment, for use in inducing MIP-1β cytokine production.
34. The composition of any preceding embodiment, for use in inducing MIP-2 cytokine production.
35. The composition of any preceding embodiment, for use in inducing IL-10 cytokine production.
36. The composition of any preceding embodiment, for use in inducing IL-22 cytokine production.
37. The composition of any preceding embodiment, for use in inducing IL-5 cytokine production.
38. The composition of any preceding embodiment, for use in inducing IL-18 cytokine production.
39. The composition of any preceding embodiment, for use in inducing IL-23 cytokine production.
40. The composition of any preceding embodiment, for use in inducing CXCL1 cytokine production.
41. The composition of any preceding embodiment, for use in inducing IL-6 cytokine production.
42. The composition of any of embodiments 1 or 4-41, wherein the composition is for use in the therapy of a viral infection, bacterial infection, fungal infection, parasitic infection or a tumour.
43. The composition of embodiment 42, wherein the composition is for use in the therapy of an influenza virus infection.
44. The composition of any of embodiments 2, 4-12, or 18-41, wherein the composition is for use in treating, preventing or delaying B cell immunosenescence.
45. The composition of any of embodiments 2, 4-12, 18-41 or 44, wherein the composition is for use in the therapy of a cardiovascular disease; a neurodegenerative disease, such as Alzheimer's disease or Parkinson's disease; cancer; type 2 diabetes; or an autoimmune disease; by treating, preventing or delaying immunosenescence.
46. The composition of any of embodiments 3-12 or 17-41, wherein the composition is for use in the therapy of cancer by enhancing CAR-T.
47. The composition of embodiment 46, wherein the cancer is chronic lymphocytic leukaemia.
48. A vaccine composition comprising a bacterial strain of the genus Parabacteroides and one or more antigens.
49. A vaccine composition according to embodiment 48, comprising one or more pathogen antigens or tumour antigens.
50. The composition of embodiment 48 or 49, wherein the bacterial strain belongs to the species Parabacteroides distasonis, Parabacteroides goldsteinii or Parabacteroides merdae.
51. The composition of any of embodiments 48-50, wherein the bacterial strain has a 16s rRNA gene sequence that is at least 95%, 96%, 97%, 98%, 99%, 99.5% or 99.9% identical to SEQ ID NO: 1, 2, 3, 4, 5, 6, 7, 8, 9, 10, 11, 12, 13, 14, 15, 16, 17, 18, 19, 20, 21, 22 or 23, or wherein the bacterial strain has a 16s rRNA gene sequence represented by SEQ ID NO: 1, 2, 3, 4, 5, 6, 7, 8, 9, 10, 11, 12, 13, 14, 15, 16, 17, 18, 19, 20, 21, 22 or 23.
52. The composition of any of embodiments 48-51, wherein the bacterial strain has a 16s rRNA gene sequence that is at least 95%, 96%, 97%, 98%, 99%, 99.5% or 99.9% identical to SEQ ID NO:9 or wherein the bacterial strain has a 16s rRNA gene sequence represented by SEQ ID NO:9.
53. The composition of embodiment 52, wherein the bacterial strain is the strain deposited under accession number 42382 at NCIMB.
54. The composition of any of embodiments 48-51, wherein the bacterial strain has a 16s rRNA gene sequence that is at least 98%, 99%, 99.5% or 99.9% identical to SEQ ID NO: 9, 12, 13, 16, 17, or 19, or wherein the bacterial strain has a 16s rRNA gene sequence represented by SEQ ID NO: 9, 12, 13, 16, 17, or 19.
55. The composition of any of embodiments 48-51, wherein the bacterial strain has a 16s rRNA gene sequence that is at least 98%, 99%, 99.5% or 99.9% identical to SEQ ID NO: 10 or 11, or wherein the bacterial strain has a 16s rRNA gene sequence represented by SEQ ID NO: 10 or 11.
56. The composition of embodiment 55, wherein the bacterial strain is the strain deposited under either accession number DSMZ19448 or DSMZ29187.
57. The composition of any of embodiments 48-51, wherein the bacterial strain has a 16s rRNA gene sequence that is at least 98%, 99%, 99.5% or 99.9% identical to SEQ ID NO: 18, or wherein the bacterial strain has a 16s rRNA gene sequence represented by SEQ ID NO: 18.
58. The composition of any of embodiments 48, 49 or 51, wherein the bacterial strain has a 16s rRNA gene sequence that is at least 98%, 99%, 99.5% or 99.9% identical to SEQ ID NO: 15, or wherein the bacterial strain has a 16s rRNA gene sequence represented by SEQ ID NO: 15.
59. The composition of any of embodiments 49-58, wherein the composition comprises one or more pathogen antigens.
60. The composition of claim 59, wherein the one or more pathogen antigens are selected from viral antigens, such as viral surface proteins; bacterial antigens, such as protein and/or saccharide antigens; fungal antigens; and parasite antigens.
61. The composition of embodiment 59 or 60, wherein the one or more pathogen antigens are from any of the following pathogens: influenza virus, HIV, hookworm, hepatitis B virus, herpes simplex virus, rabies, respiratory syncytial virus, cytomegalovirus, Staphylococcus aureus, chlamydia, SARS coronavirus, varicella zoster virus, Streptococcus pneumoniae, Neisseria meningitidis, Mycobacterium tuberculosis, Bacillus anthracis, Epstein Barr virus, or human papillomavirus.
62. The composition of embodiment 61, wherein the composition comprises one or more influenza virus antigens.
63. The composition of any of embodiments 48-58, wherein the composition comprises one or more antigens selected from neoantigens, glycoprotein antigens, lipoglycan antigens, archaea antigens, melanoma antigen E (MAGE), Carcinoembryonic antigen (CEA), MUC-1, HER2, sialyl-Tn (STn), human telomerase reverse transcriptase (hTERT), Wilms tumour gene (WT1), CA-125, prostate-specific antigen (PSA), oncoproteins, amyloid-beta, Tau, PCSK9 or habit forming substances such as alcohol or opiates.
64. The composition of any of embodiments 48-63, wherein the composition is for oral administration.
65. The composition of any of embodiments 48-64, wherein the composition comprises one or more pharmaceutically acceptable excipients or carriers.
66. The composition of any of embodiments 48-65, wherein the bacterial strain is lyophilised.
67. The composition of any of embodiments 48-66, wherein the bacterial strain expresses a heterologous antigen, such as a pathogen antigen or a tumour antigen.
68. The composition of any of embodiments 48-67, for use in medicine.
69. The composition of embodiment 68, for use in increasing the expression level and/or activity of MCP-1 and/or the expansion of B-cells.
70. The composition of embodiment 69, wherein the B cells include CD19⁺CD3⁻ B cells.
71. The composition of any of embodiments 68-70, for use in inducing TNF-α cytokine production.
72. The composition of any of embodiments 68-71, for use in inducing IL-1B cytokine production.
73. The composition of any of embodiments 68-72, for use in inducing IL-2 cytokine production.
74. The composition of any of embodiments 68-73, for use in inducing GM-CSF cytokine production.
75. The composition of any of embodiments 68-74, for use in inducing IFN-γ cytokine production.
76. The composition of any of embodiments 68-75, for use in inducing IL-27 cytokine production.
77. The composition of any of embodiments 68-76, for use in inducing IFN-γ cytokine production.
78. The composition of any of embodiments 68-77, for use in inducing IL-27 cytokine production.
79. The composition of any of embodiments 68-78, for use in inducing IP-10 cytokine production.
80. The composition of any of embodiments 68-79, for use in inducing RANTES cytokine production.
81. The composition of any of embodiments 68-80, for use in inducing MIP-1α cytokine production.
82. The composition of any of embodiments 68-81, for use in inducing MIP-1β cytokine production.
83. The composition of any of embodiments 68-82, for use in inducing MIP-2 cytokine production.
84. The composition of any of embodiments 68-83, for use in inducing IL-10 cytokine production.
85. The composition of any of embodiments 68-84, for use in inducing IL-22 cytokine production.
86. The composition of any of embodiments 68-85, for use in inducing IL-5 cytokine production.
87. The composition of any of embodiments 68-86, for use in inducing IL-18 cytokine production.
88. The composition of any of embodiments 68-87, for use in inducing IL-23 cytokine production.
89. The composition any of embodiments 68-88, for use in inducing CXCL1 cytokine production.
90. The composition of any preceding embodiment, for use in inducing IL-6 cytokine production.
91. The composition of any of embodiments 68-90, for use in vaccination.
92. The composition of any of embodiments 68-91, wherein the composition is for use in the therapy of a viral infection, bacterial infection, fungal infection, parasitic infection or a tumour.
93. The composition of embodiment 92, wherein the composition is for use in the therapy of an influenza virus infection.
94. The composition of any of embodiments 68-90, wherein the composition is for use in treating, preventing or delaying immunosenescence.
95. The composition of embodiment 94, wherein the immunosenescence is B cell immunosenescence.
96. The composition of any of embodiments 94 or 95, wherein the composition is for use in the therapy of a cardiovascular disease; a neurodegenerative disease, such as Alzheimer's disease or Parkinson's disease; cancer; type 2 diabetes; or an autoimmune disease; by treating, preventing or delaying immunosenescence.
97. The composition of any of embodiments 68-90, wherein the composition is for use in the therapy of cancer by enhancing CAR-T.
98. The composition of embodiment 97, wherein the cancer is chronic lymphocytic leukaemia.
99. The composition of any of embodiments 1-47 or 68-98, for use in an immunocompromised subject.
100. The composition of any of embodiments 1-47 or 68-99, for use in an immunosuppressed subject.
101. The composition according to embodiment 99 or 100, wherein the subject has an elevated number of regulatory T cells (Tregs) within a lymph node, compared to a lymph node of a subject free of disease.
102. The composition according to any of embodiments 99-101, for use in a subject with cancer, wherein the subject has an elevated number of regulatory T cells (Tregs) within a lymph node, such as a metastatic lymph node, compared to a lymph node of a subject free of cancer.
103. The composition according to any of embodiments 99-102, wherein the subject has an elevated number of Tregs within a volume of peripheral blood mononuclear cells (PBMCs), compared to the same volume of PBMCs from a subject free of disease.
104. The composition according to any of embodiments 99-103, for use in a subject with cancer, wherein the subject has an elevated number of Tregs within a volume of peripheral blood mononuclear cells (PBMCs), compared to the same volume of PBMCs from a subject free of cancer.
105. The composition according to any of embodiments 99-104, wherein the subject has an elevated number of myeloid dendritic cells (mDCs) within a volume of PBMCs, compared to the same volume of PBMCs from a subject free of disease.
106. The composition according to any of embodiments 99-105, for use in a subject with cancer, wherein the subject has an elevated number of myeloid dendritic cells (mDCs) within a volume of PBMCs, compared to the same volume of PBMCs from a subject free of cancer.
107. The composition according to any of embodiments 99-106, wherein the subject has an elevated number of plasmacytoid dendritic cells (pDCs) within a volume of PBMCs, compared to the same volume of PBMCs from a subject free of disease.
108. The composition according to any of embodiments 99-207, for use in a subject with cancer, wherein the subject has an elevated number of plasmacytoid dendritic cells (pDCs) within a volume of PBMCs, compared to the same volume of PBMCs from a subject free of cancer.

### BRIEF DESCRIPTION OF DRAWINGS

FIGS. 1A-1F: Increased percentage of immune cells by NCIMB 42382 treatment.

FIGS. 2A-2C: Gating strategy used to analyse the different population of immune cells (CD4, CD8 and CD19+ cells) by Flow Cytometry for the data presented in FIG. 1.

FIG. 3: Increased secretion of MCP-1 by NCIMB 42382 treatment.

FIGS. 4A-4B: Induction of TNF-α secretion from HT29 cells by (FIG. 4A) NCIMB 42382 with conditioned media and (FIG. 4B) NCIMB 42382 alone.

FIG. 5: Fermentation profile of NCIMB 42382 obtained using the (left) Rapid ID 32 A and (right) API 50 CHL systems.

FIG. 6: Splenocyte proliferation following treatment with Parabacteroides strains (“YCFA”=YCFA+).

FIGS. 7A-7W: Cytokine secretion from splenocytes following treatment with various Parabacteroides strains—(FIG. 7A) TNF-α, (FIG. 7B) IL-113, (FIG. 7C) IL-2, (FIG. 7D) GM-CSF, (FIG. 7E) IFN-γ, (FIG. 7F) IL-27, (FIG. 7G) IL-10, (FIG. 7H) IL-6, (FIG. 7I) MIP-2, (FIG. 7J) MIP-1α, (FIG. 7K) MIP-113, (FIG. 7L) IL-22, (FIG. 7M) RANTES, (FIG. 7N) IP-10, (FIG. 7O) IL-4, (FIG. 7P), IL-5, (FIG. 7Q), IL-18, (FIG. 7R) IL-23, (FIG. 7S) IL-9, (FIG. 7T) CXCL1, (FIG. 7U) MCP-3, (FIG. 7V) MCP-1 and (FIG. 7W) IL-17A (“YCFA”=YCFA+).

FIGS. 8A-8I: Cytokine secretion from splenocytes following treatment with various Parabacteroides strains—(FIG. 8A) strain ref. 9 (P. distasonis), (FIG. 8B) strain ref. 10 (P. johnsonii), (FIG. 8C) strain ref 7 (P. merdae), (FIG. 8D) strain ref. 11 (Parabacteroides sp.), (FIG. 8E) strain ref 2 (P. distasonis), (FIG. 8F) strain ref 12 (Parabacteroides sp.), (FIG. 8G) strain ref 13 (Parabacteroides sp.), (FIG. 8H) strain ref 14 (Parabacteroides sp.) and (FIG. 8I) strain ref 15 (Parabacteroides sp.).

### DISCLOSURE OF THE INVENTION

### Bacterial Strains

The compositions of the invention comprise a strain of the genus Parabacteroides (e.g. of the species Parabacteroides distasonis, Parabacteroides goldsteinii, Parabacteroides merdae or Parabacteroides Parabacteroides johnsonii). The examples demonstrate that such bacterial strains elicit immunological responses which are strongly associated with vaccine adjuvancy. The preferred bacterial strains of the invention are those belonging to the species Parabacteroides distasonis, Parabacteroides goldsteinii and Parabacteroides merdae, particularly Parabacteroides distasonis. The preferred bacterial strain of the invention is the bacterium deposited under accession number NCIMB 42382.

The Parabacteroides resemble the Bacteroides and are Gram-negative, obligately anaerobic, non-spore-forming, non-motile and rod-shaped, and 0.8-1.6×1.2-12 μm in size. Parabacteroides distasonis is one of the most common species in human faeces. The type strain of P. distasonis is JCM 5825^{T} (=CCUG 4941^{T}=DSM 20701^{T}=ATCC 8503^{T}) The GenBank/EMBL/DDBJ accession numbers for the 16S rRNA gene sequences of P. distasonis strains JCM 5825T, JCM 13400, JCM 13401, JCM 13402, JCM 13403 and JCM 13404 and P. merdae strains JCM 9497T and JCM 13405 are AB238922-AB238929, respectively (disclosed herein as SEQ ID NOs:1-8). Exemplary strains are also described in [19].

The Parabacteroides distasonis bacterium deposited under accession number NCIMB 42382 was tested in the Examples and is also referred to herein as strain 755 (or NCIMB 42382 or strain NCIMB 42382). The strain was isolated from the digestive tract of a healthy human donor. A 16S rRNA gene sequence for the 755 strain that was tested is provided in SEQ ID NO:9. Strain 755 was deposited with the international depositary authority NCIMB, Ltd. (Ferguson Building, Aberdeen, AB21 9YA, Scotland) by GT Biologics Ltd. (Life Sciences Innovation Building, Aberdeen, AB25 2ZS, Scotland) on 12 Mar. 2015 as “Parabacteroides sp 755” and was assigned accession number NCIMB 42382. GT Biologics Ltd. subsequently changed its name to 4D Pharma Research Limited.

WO 2016/203220 describes administration of strain 755 to mice and shows that it can affect disease processes outside of the gut (such as asthma and arthritis). Furthermore, no morbidity or mortality was observed as a result of treatment with the bacterial strain, thus indicating its safety for therapeutic applications without needing to manipulate the naturally-occurring strain.

A genome sequence for strain NCIMB 42382 is provided in SEQ ID NO:10 of WO 2016/203220. This sequence was generated using the PacBio RS II platform.

The Parabacteroides goldsteinii strains deposited under accession numbers DSMZ19448 and DSMZ29187 were tested in the Examples. A 16s rRNA gene sequence for strain DSMZ19448 is provided in SEQ ID NO: 10. A 16s rRNA gene sequence for strain DSMZ29187 is provided in SEQ ID NO: 11. The strains were deposited with the DSMZ—German Collection of Microorganisms and Cell Cultures GmbH (Inhoffenstr. 7B 38124 Braunschweig, Germany) and are publically available.

The following Parabacteroides strains were also tested in the Examples: strain ref 1 (Parabacteroides distasonis), strain ref 2 (Parabacteroides distasonis), strain ref 3 (Parabacteroides sp.), strain ref 4 (Parabacteroides johnsonii), strain ref 5 (Parabacteroides distasonis), strain ref 6 (Parabacteroides distasonis), strain ref 7 (Parabacteroides merdae), strain ref 8 (Parabacteroides distasonis), strain ref 9 (Parabacteroides distasonis), strain ref 10 (Parabacteroides johnsonii), strain ref 11 (Parabacteroides sp.), strain ref 12 (Parabacteroides sp.), strain ref 13 (Parabacteroides sp.), strain ref 14 (Parabacteroides sp.), strain ref 15 (Parabacteroides sp.). A 16s rRNA gene sequence for strain ref 1 (P. distasonis) is provided in SEQ ID NO: 12. A 16s rRNA gene sequence for strain ref 2 (P. distasonis) is provided in SEQ ID NO: 13. A 16s rRNA gene sequence for strain ref 3 (Parabacteroides sp.) is provided in SEQ ID NO: 14. A 16s rRNA gene sequence for strain ref 4 (P. johnsonii) is provided in SEQ ID NO: 15. A 16s rRNA gene sequence for strain ref 5 (P. distasonis) is provided in SEQ ID NO: 16. A 16s rRNA gene sequence for strain ref 6 (P. distasonis) is provided in SEQ ID NO: 17. A 16s rRNA gene sequence for strain ref 7 (P. merdae) is provided in SEQ ID NO: 18. A 16s rRNA gene sequence for strain ref 9 (P. distasonis) is provided in SEQ ID NO: 19. A 16s rRNA gene sequence for strain ref 11 (Parabacteroides sp) is provided in SEQ ID NO: 20. A 16s rRNA gene sequence for strain ref 12 (Parabacteroides sp) is provided in SEQ ID NO: 21. A 16s rRNA gene sequence for strain ref 14 (Parabacteroides sp) is provided in SEQ ID NO: 22. A 16s rRNA gene sequence for strain ref. 15 (Parabacteroides sp) is provided in SEQ ID NO: 23.

Bacterial strains closely related to the strain tested in the examples are also expected to be effective as vaccine adjuvants. In certain embodiments, the bacterial strain for use in the invention has a 16s rRNA gene sequence that is (in increasing preference) at least 90%, 91%, 92%, 93%, 94%, 95%, 96%, 97%, 98%, 99%, 99.5% or 99.9% identical to the 16s rRNA gene sequence of a bacterial strain of Parabacteroides distasonis. The bacterial strain for use in the invention may have a 16s rRNA gene sequence that is (in increasing preference) at least 90%, 91%, 92%, 93% or 94% identical to SEQ ID NO: 1, 2, 3, 4, 5, 6, 7, 8, 9, 10, 11, 12, 13, 14, 15, 16, 17, 18, 19, 20, 21, 22 or 23, preferably to SEQ ID NO: 9. Preferably, the bacterial strain for use in the invention has a 16s rRNA gene sequence that is (in increasing preference) at least 95%, 96%, 97%, 98%, 99%, 99.5% or 99.9% identical to SEQ ID NO: 1, 2, 3, 4, 5, 6, 7, 8, 9, 10, 11, 12, 13, 14, 15, 16, 17, 18, 19, 20, 21, 22 or 23. Preferably, the sequence identity is to SEQ ID NO:9. Preferably, the bacterial strain for use in the invention has the 16s rRNA gene sequence represented by SEQ ID NO:9. Most preferably, the bacterial strain for use in the invention is of the Parabacteroides distasonis strain deposited under accession number NCIMB 42382.

In embodiments where the bacterial strain used in compositions of the invention is of the species Parabacteroides distasonis, preferred strains have a 16s rRNA gene sequence that is (in increasing preference) at least 98%, 99%, 99.5% or 99.9% identical to SEQ ID NO: 9, 12, 13, 16, 17, or 19, preferably to SEQ ID NO: 9. More preferably, such preferred strains have the 16s rRNA gene sequence represented by SEQ ID NO: 9, 12, 13, 16, 17, or 19, in particular SEQ ID NO: 9. Most preferably, the bacterial strain is the strain of Parabacteroides distasonis deposited under accession number NCIMB 43382.

In embodiments where the bacterial strain used in compositions of the invention is of the species Parabacteroides goldsteinii, preferred strains have a 16s rRNA gene sequence that is (in increasing preference) at least 98%, 99%, 99.5% or 99.9% identical to SEQ ID NO: 10 or 11, or more preferably have the 16s rRNA gene sequence represented by SEQ ID NO: 10 or 11, or most preferably are either of the Parabacteroides goldsteinii strains deposited under accession numbers DSMZ19448 and DSMZ29187.

In embodiments where the bacterial strain used in compositions of the invention is of the species Parabacteroides merdae, preferred strains have a 16s rRNA gene sequence that is (in increasing preference) at least 98%, 99%, 99.5% or 99.9% identical to SEQ ID NO: 18 or more preferably have the 16s rRNA gene sequence represented by SEQ ID NO: 18.

In embodiments where the bacterial strain used in compositions of the invention is of the species Parabacteroides johnsonii, preferred strains have a 16s rRNA gene sequence that is (in increasing preference) at least 98%, 99%, 99.5% or 99.9% identical to SEQ ID NO: 15, or more preferably have the 16s rRNA gene sequence represented by SEQ ID NO: 15.

In preferred embodiments, the composition of the invention comprises live bacteria. In preferred embodiments, the composition of the invention comprises live bacteria in an active state, preferably lyophilised.

In preferred embodiments, the bacterial strain of the invention increases the secretion of MCP-1, for example by PBMCs such as described in the examples. In a preferred embodiment, the composition of the invention comprises a bacteria that increases the expression of MCP-1 and is for use as a vaccine adjuvant. In a preferred embodiment, the composition of the invention comprises a bacterial strain that increases the expression of MCP-1 and is for use in enhancing a cell therapy, such as CAR-T.

In preferred embodiments, the bacterial strain of the invention increases the expansion of B-cells, for example by PBMCs such as described in the examples. In a preferred embodiment, the composition of the invention comprises a bacterial strain that increases the expansion of B-cells and is for use as a vaccine adjuvant. In a preferred embodiment, the composition of the invention comprises a bacterial strain that increases the expansion of B-cells and is for use in enhancing a cell therapy, such as CAR-T. In a preferred embodiment, the composition of the invention comprises a bacterial strain that increases the expansion of B-cells and is for use in treating, preventing or delaying immunosenescence.

In preferred embodiments, the bacterial strain of the invention increases the proliferation of splenocytes, for example as described in the examples. In a preferred embodiment, the composition of the invention comprises a bacterial strain that increases the proliferation of splenocytes and is for use as a vaccine adjuvant. In a preferred embodiment, the composition of the invention comprises a bacterial strain that increases the proliferation of splenocytes and is for use in enhancing a cell therapy, such as CAR-T. In a preferred embodiment, the composition of the invention comprises a bacterial strain that increases the proliferation of splenocytes and is for use in treating, preventing or delaying immunosenescence.

In preferred embodiments, the bacterial strain of the invention increases the production of one or more, preferably all of, the cytokines TNF-α, IL-1β, IL-27, IL-10, MIP-2, MIP-1α, MIP-1β, IL-22, IL-5, IL-18, IL-23, CXCL1, IL-2, GM-CSF, IFN-γ, IL-6, IP-10 and/or RANTES, for example by splenocytes e.g. such as described in the examples. In a preferred embodiment, the composition of the invention comprises a bacterial strain that increases the production of one or more, preferably all of, the cytokines TNF-α, IL-1β, IL-27, IL-10, MIP-2, MIP-1α, MIP-1β, IL-22, IL-5, IL-18, IL-23, CXCL1, IL-2, GM-CSF, IFN-γ, IL-6, IP-10 and/or RANTES and is for use as a vaccine adjuvant. In a preferred embodiment, the composition of the invention comprises a bacterial strain that increases the production of one or more, preferably all of, the cytokines TNF-α, IL-1β, IL-27, IL-10, MIP-2, MIP-1α, MIP-1β, IL-22, IL-5, IL-18, IL-23, CXCL1, IL-2, GM-CSF, IFN-γ, IL-6, IP-10 and/or RANTES and is for use in enhancing a cell therapy, such as CAR-T. In a preferred embodiment, the composition of the invention comprises a bacterial strain that increases the production of one or more, preferably all of, the cytokines TNF-α, IL-1β, IL-27, IL-10, MIP-2, MIP-1α, MIP-1β, IL-22, IL-5, IL-18, IL-23, CXCL1, IL-2, GM-CSF, IFN-γ, IL-6, IP-10 and/or RANTES and is for use in treating, preventing or delaying immunosenescence.

In certain embodiments, a composition of the invention comprises a biotype of the bacterium deposited under accession number NCIMB 42382. Bacterial strains that are biotypes of the bacterium deposited under accession number NCIMB 42382 are also expected to be useful as vaccine adjuvants. A biotype will have comparable activity to the original NCIMB 42382 strain. A biotype is a closely related strain that has the same or very similar physiological and biochemical characteristics.

A biotype will elicit comparable effects on the expression of MCP-1 and/or expansion of B-cells to the effects shown in the examples, which may be identified by using the culturing and administration protocols described in the examples. For example, a biotype of strain NCIMB 42382 may increase the percentage of B-cells (e.g. CD19+CD3− cells) in a population of peripheral blood mononuclear cells (PBMCs), e.g. to greater than 40% of the cell population (e.g. to a mean of greater than 40% of the cell population based on 5 repetitions), which may be determined using the culturing and administration protocols described in the examples. For example, in addition or alternatively, a biotype of strain NCIMB 42382 may increase expression of MCP-1 by PBMCs, e.g. to greater than 1000 pg/ml MCP-1 protein of cell-free co-culture supernatant, which may be determined using the culturing and administration protocols described in the examples.

In addition or alternatively, a biotype of strain NCIMB 42382 will increase the proliferation of splenocytes, e.g. to a greater extent than untreated splenocytes or splenocytes treated with a control media (e.g. YCFA+media), which may be determined using an assay which measures the conversion of 3-[4,5-dimethylthiazole-2-yl]-2,5-diphenyltetrazolium bromide (MTT) to MTT-formazan, e.g. by colourimetric detection of MTT-formazan (e.g. as in Example 10). In addition or alternatively, a biotype of strain NCIMB 42382 will increase the production of one or more, preferably all of, the cytokines TNF-α, IL-1β, IL-27, IL-10, MIP-2, MIP-1α, MIP-1β, IL-22, IL-5, IL-18, IL-23, CXCL1, IL-2, GM-CSF, IFN-γ, IL-6, IP-10 and/or RANTES from splenocytes, e.g. to a greater extent than untreated splenocytes or splenocytes treated with a control media (e.g. YCFA+media), which may be determined by a cytokine immunoassay (e.g. the 26-plex Mouse ProcartaPlex™ multiplex immunoassay from Thermo Fischer Scientific as used in Examples 11 and 12).

Strains that are biotypes of a bacterium deposited under accession number NCIMB 42382 and that are suitable for use in the invention may be identified by sequencing other nucleotide sequences for a bacterium deposited under accession number NCIMB 42382. For example, substantially the whole genome may be sequenced and a biotype strain for use in the invention may have at least 95%, 96%, 97%, 98%, 99%, 99.5% or 99.9% sequence identity across at least 80% of its whole genome (e.g. across at least 85%, 90%, 95% or 99%, or across its whole genome). For example, in some embodiments, a biotype strain has at least 98% sequence identity across at least 98% of its genome or at least 99% sequence identity across 99% of its genome. Other suitable sequences for use in identifying biotype strains may include hsp60 or repetitive sequences such as BOX, ERIC, (GTG)₅, or REP [20].

Biotype strains may have such sequences with at least 95%, 96%, 97%, 98%, 99%, 99.5% or 99.9% sequence identity to the corresponding sequence of a bacterium deposited under accession number NCIMB 42382. In some embodiments, a biotype strain may have a 16S rRNA gene sequence with at least 95%, 96%, 97%, 98%, 99%, 99.5% or 99.9% sequence identity to the corresponding sequence of a bacterium deposited under accession number NCIMB 42382. In some embodiments, a biotype strain may comprises a 16S rRNA gene sequence that is at least 99% identical (e.g. at least 99.5% or at least 99.9% identical) to SEQ ID NO:9. In some embodiments, a biotype strain has the 16S rRNA gene sequence of SEQ ID NO:9.

In certain embodiments, the bacterial strain for use in the invention has a genome with sequence identity to SEQ ID NO:10 of WO 2016/203220. In preferred embodiments, the bacterial strain for use in the invention has a genome with at least 90% sequence identity (e.g. at least 92%, 94%, 95%, 96%, 97%, 98%, 99% or 100% sequence identity) to SEQ ID NO:10 of WO 2016/203220 across at least 60% (e.g. at least 65%, 70%, 75%, 80%, 85%, 95%, 96%, 97%, 98%, 99% or 100%) of SEQ ID NO:10 of WO 2016/203220. For example, the bacterial strain for use in the invention may have a genome with at least 90% sequence identity to SEQ ID NO:10 of WO 2016/203220 across 70% of SEQ ID NO:10 of WO 2016/203220, or at least 90% sequence identity to SEQ ID NO:10 of WO 2016/203220 across 80% of SEQ ID NO:10 of WO 2016/203220, or at least 90% sequence identity to SEQ ID NO:10 of WO 2016/203220 across 90% of SEQ ID NO:10 of WO 2016/203220, or at least 90% sequence identity to SEQ ID NO:10 of WO 2016/203220 across 100% of SEQ ID NO:10 of WO 2016/203220, or at least 95% sequence identity to SEQ ID NO:10 of WO 2016/203220 across 70% of SEQ ID NO:10 of WO 2016/203220, or at least 95% sequence identity to SEQ ID NO:10 of WO 2016/203220 across 80% of SEQ ID NO:10 of WO 2016/203220, or at least 95% sequence identity to SEQ ID NO:10 of WO 2016/203220 across 90% of SEQ ID NO:10 of WO 2016/203220, or at least 95% sequence identity to SEQ ID NO:10 of WO 2016/203220 across 100% of SEQ ID NO:10 of WO 2016/203220, or at least 98% sequence identity to SEQ ID NO:10 of WO 2016/203220 across 70% of SEQ ID NO:10 of WO 2016/203220, or at least 98% sequence identity to SEQ ID NO:10 of WO 2016/203220 across 80% of SEQ ID NO:10 of WO 2016/203220, or at least 98% sequence identity to SEQ ID NO:10 of WO 2016/203220 across 90% of SEQ ID NO:10 of WO 2016/203220, or at least 98% sequence identity to SEQ ID NO:10 of WO 2016/203220 across 100% of SEQ ID NO:10 of WO 2016/203220.

Alternatively, strains that are biotypes of a bacterium deposited under accession number NCIMB 42382 and that are suitable for use in the invention may be identified by using the accession number NCIMB 42382 deposit, and restriction fragment analysis and/or PCR analysis, for example by using fluorescent amplified fragment length polymorphism (FAFLP) and repetitive DNA element (rep)-PCR fingerprinting, or protein profiling, or partial 16S or 23 s rDNA sequencing. In preferred embodiments, such techniques may be used to identify other Parabacteroides strains.

In certain embodiments, strains that are biotypes of a bacterium deposited under accession number NCIMB 42382 and that are suitable for use in the invention are strains that provide the same pattern as a bacterium deposited under accession number NCIMB 42382 when analysed by amplified ribosomal DNA restriction analysis (ARDRA), for example when using Sau3AI restriction enzyme (for exemplary methods and guidance see, for example [21]).

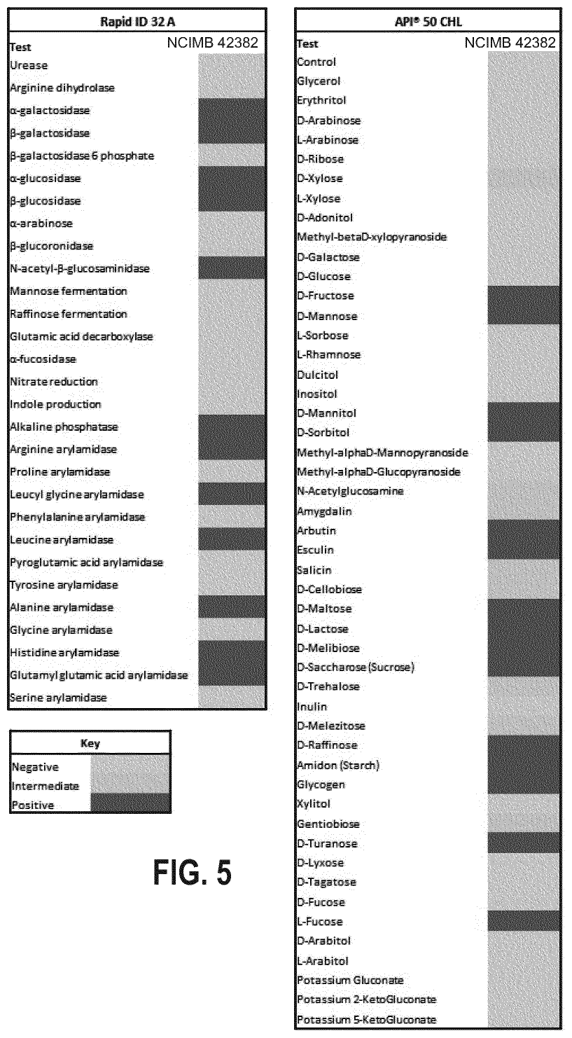

Alternatively, biotype strains are identified as strains that have the same carbohydrate fermentation patterns as a bacterium deposited under accession number NCIMB 42382 (see Example 4 and FIG. 5). Alternatively, biotype strains are identified as strains that have the same amino acid fermentation patterns as the bacterium deposited under accession number NCIMB 42382 (see Example 4 and FIG. 5).

In preferred embodiments, the biotype bacterial strain (in particular, a Parabacteroides distasonis bacterial strain) used in the invention exhibits enzymatic activity for one or more, such as (in increasing preference) 2, 3, 4 or all 5 of: α-galactosidase, β-galactosidase, α-glucosidase, β-glucosidase and alkaline phosphatase, for example when cultured in an appropriate suspension medium (such as API suspension medium) at 37° C. for 4 hours. The biotype bacterial strain (in particular, a Parabacteroides distasonis bacterial strain) used in the invention is preferably able to ferment one or more, such as (in increasing preference) 2, 3, 4, 5 or all 6 of: arginine, leucyl-glycine, leucine, alanine, histidine and glutamyl glutamic acid, for example when cultured in an appropriate suspension medium (such as API suspension medium) at 37° C. for 4 hours. The biotype bacterial strain (in particular, a Parabacteroides distasonis bacterial strain) used in the invention is more preferably able to ferment one or more, such as (in increasing preference) 2, 3, 4, 5 or all 6 of: arginine, leucyl-glycine, leucine, alanine, histidine and glutamyl glutamic acid and exhibits enzymatic activity for one or more, such as (in increasing preference) 2, 3, 4 or all 5 of: α-galactosidase, β-galactosidase, α-glucosidase, β-glucosidase and alkaline phosphatase, for example when cultured in an appropriate suspension medium (such as API suspension medium) at 37° C. for 4 hours. Any suitable assay known in the art may be used to assess the ability of a bacterium to ferment a carbohydrate source or amino acid. Preferably, the Rapid ID 32A analysis is used (preferably using the Rapid ID 32A system from bioMérieux).

In alternative preferred embodiments, the biotype bacterial strain (in particular, a Parabacteroides distasonis bacterial strain) used in the invention is able to ferment one or more, such as (in increasing preference) 2, 3, 4, 5, 6, 7, 8, 9, 10, 11, 12, 13, 14 or all 15 of: fructose, mannose, mannitol, sorbitol, arbutin, esculin, maltose, lactose, melibiose, sucrose, raffinose, starch, glycogen, turanose and fucose. The biotype bacterial strain (in particular, a Parabacteroides distasonis bacterial strain) used in the invention preferably furthermore exhibits intermediate fermentation of one or more, such as (in increasing preference) 2, 3, 4, 5, 6, 7 or all 8 of: xylose, N-acetylglucosamine, amygdalin, salicin, cellobiose, trehalose, melezitose and gentiobiose. In such embodiments, any suitable assay known in the art may be used to assess the ability of a bacterium to ferment a carbohydrate source. Preferably, the API 50 CH analysis is used (preferably using the API 50 CH system from bioMérieux).

An especially preferred biotype bacterial strain (in particular, a Parabacteroides distasonis bacterial strain) used in the invention (i) exhibits enzymatic activity for α-galactosidase, β-galactosidase, α-glucosidase, β-glucosidase and alkaline phosphatase; (ii) is able to ferment arginine, leucyl-glycine, leucine, alanine, histidine and glutamyl glutamic acid; and (iii) is able to ferment fructose, mannose, mannitol, sorbitol, arbutin, esculin, maltose, lactose, melibiose, sucrose, raffinose, starch, glycogen, turanose and fucose. The biotype bacterial strain preferably furthermore (iv) exhibits intermediate fermentation of xylose, N-acetylglucosamine, amygdalin, salicin, cellobiose, trehalose, melezitose and gentiobiose. (i) and (ii) are preferably assessed when the bacterial strain is cultured in an appropriate suspension medium (such as API suspension medium) at 37° C. for 4 hours, and assessed by Rapid ID 32A analysis (preferably using the Rapid ID 32A system from bioMérieux). (iii) and (iv) are preferably assessed by API 50 CH analysis (preferably using the API 50 CH system from bioMérieux).

Other Parabacteroides strains that are useful in the compositions and methods of the invention, such as biotypes of a bacterium deposited under accession number NCIMB 42382, may be identified using any appropriate method or strategy, including the assays described in the examples. In particular, bacterial strains that have similar growth patterns, metabolic type and/or surface antigens to a bacterium deposited under accession number NCIMB 42382 may be useful in the invention.

In certain embodiments, a composition of the invention comprises a derivative of the bacterium deposited under accession number NCIMB 42382. A derivative of the strain deposited under accession number NCIMB 42382 may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable vaccine adjuvant activity to the original NCIMB 42382 strain. A derivative of the NCIMB 42382 strain will generally be a biotype of the NCIMB 42382 strain.

A derivative strain will elicit comparable vaccine adjuvant effects to the effects shown in the examples, which may be identified by using the culturing and administration protocols described in the examples. In particular, a derivative strain will elicit an effect on MCP-1 expression and B-cell expansion comparable to those of a bacterium deposited under accession number NCIMB 42382. A derivative of the NCIMB 42382 strain will generally be a biotype of the NCIMB 42382 strain. For example, a derivative of strain NCIMB 42382 may increase the percentage of B-cells (e.g. CD19+CD3− cells) in a population of peripheral blood mononuclear cells (PBMCs), e.g. to greater than 40% of the cell population (e.g. to a mean of greater than 40% of the cell population based on 5 repetitions), which may be determined using the culturing and administration protocols described in the examples. For example, in addition or alternatively, a derivative of strain NCIMB 42382 may increase expression of MCP-1 by PBMCs, e.g. to greater than 1000 pg/ml MCP-1 protein of cell-free co-culture supernatant, which may be determined using the culturing and administration protocols described in the examples.

In addition or alternatively, a derivative of strain NCIMB 42382 will increase the proliferation of splenocytes, e.g. to a greater extent than untreated splenocytes or splenocytes treated with a control media (e.g. YCFA+media), which may be determined using an assay which measures the conversion of 3-[4,5-dimethylthiazole-2-yl]-2,5-diphenyltetrazolium bromide (MTT) to MTT-formazan, e.g. by colourimetric detection of MTT-formazan (e.g. as in Example 10). In addition or alternatively, a derivative of strain NCIMB 42382 will increase the production of one or more, preferably all of, the cytokines TNF-α, IL-1β, IL-27, IL-10, MIP-2, MIP-1α, MIP-1β, IL-22, IL-5, IL-18, IL-23, CXCL1, IL-2, GM-CSF, IFN-γ, IL-6, IP-10 and/or RANTES from splenocytes, e.g. to a greater extent than untreated splenocytes or splenocytes treated with a control media (e.g. YCFA+media), which may be determined by a cytokine immunoassay (e.g. the 26-plex Mouse ProcartaPlex™ multiplex immunoassay from Thermo Fischer Scientific as used in Examples 11 and 12).

References to cells of the Parabacteroides strain deposited under accession number NCIMB 42382 encompass any cells that have the same safety and therapeutic efficacy characteristics as the strain deposited under accession number NCIMB 42382, and such cells are encompassed by the invention. The composition can therefore comprise a Parabacteroides strain that is not the strain deposited under accession number NCIMB 42382 but has the same safety and therapeutic efficacy characteristics as the strain deposited under accession number NCIMB 42382. The safety characteristics of a strain can be established for example by testing the resistance of the strain to antibiotics, for example distinguishing between intrinsic and transmissible resistance to antibiotics. The safety characteristics of a strain can also be established by evaluating the pathogenic properties of a strain in vitro, for example the levels of toxin production. Other safety tests include testing the acute or chronic toxicity of the bacterial strain in rat and mice models. The therapeutic efficacy of a strain can be established by functional characterization of the bacterial strain in vitro and in vivo using a relevant model.

In preferred embodiments, the bacterial strains in the compositions of the invention are viable and capable of partially or totally colonising the intestine.

In certain preferred embodiments, the bacterial strain for use in the invention is able to increase the expression of MCP-1 and/or expansion of B-cells (especially B-lymphocytes) from PBMCs.

In certain preferred embodiments, the bacterial strains for use in the invention are able to increase the proliferation of splenocytes. This may be determined using an assay which measures the conversion of 3-[4,5-dimethylthiazole-2-yl]-2,5-diphenyltetrazolium bromide (MTT) to MTT-formazan, e.g. by colourimetric detection of MTT-formazan (e.g. as in Example 5).

In certain preferred embodiments, the bacterial strains for use in the invention are able to increase the production of one or more, preferably all of, TNF-α, IL-113, IL-27, IL-10, MIP-2, MIP-1α, MIP-1β, IL-22, IL-5, IL-18, IL-23, CXCL1, IL-2, GM-CSF, IFN-γ, IL-6, IP-10 and/or RANTES from splenocytes. This may be determined by a cytokine immunoassay (e.g. the 26-plex Mouse ProcartaPlex® multiplex immunoassay from Thermo Fischer Scientific as used in Examples 6 and 7).

In certain preferred embodiments, the bacterial strains for use in the invention produce acetic acid. In certain preferred embodiments, the bacterial strains for use in the invention produce propionic acid. In certain preferred embodiments, the bacterial strains for use in the invention produce acetic acid and propionic acid. The production of acetic and/or propionic acid may be determined using gas chromatography/mass spectrometry (e.g. as in Examples 8 and 9).

In some embodiments, the bacterial strain in the compositions of the invention is a bacterial strain of the genus Parabacteroides, wherein the bacterial strain is not of the strain deposited under accession number NCIMB 42382.

In some embodiments, the bacterial strain in the compositions of the invention is a bacterial strain of the species Parabacteroides distasonis, wherein the bacterial strain is not of the strain deposited under accession number NCIMB 42382.

### Therapeutic Uses

### Use as a Vaccine Adjuvant

The examples show that administration of the compositions of the invention can lead to an increase in expression of MCP-1. MCP-1 is known to be important for vaccine responses. Studies published on adjuvants like MF59 and Alum highlighted that secretion of chemokines, including MCP-1, is associated with adjuvant efficacy [22]. Chemokines have been used as vaccine adjuvants due to their ability to modulate lymphocyte development, priming and effector functions, and enhance protective immunity [23]. Additional chemokines which Parabacteroides strains have been found to upregulate include IL-5, CXCL1, IP-10, RANTES, MIP-1α, MIP-1B and MIP-2 (see the examples), similar to established vaccine adjuvants such as MF59 (which upregulates inter alia RANTES, MIP-1α, MIP-1B [57]). Furthermore Parabacteroides strains have been found to increase the production of GM-CSF from splenocytes (see the examples), which is itself used to provide an adjuvant effect for clinically-approved vaccines [58]. TNF-α, which Parabacteroides strains were found to induce the expression of from the HT29 cell line and from splenocytes in the examples, also has reported vaccine adjuvant effects [55]. Since administration of the compositions of the invention were shown to increase inter alia, MCP-1 expression, compositions of the invention may be useful as a vaccine adjuvant. In one embodiment, the compositions of the invention are for use as a vaccine adjuvant by increasing the expression level and/or activity of MCP-1. In another embodiment, compositions of the invention are for use as a vaccine adjuvant by increasing the expression level and/or activity (preferably expression level) of one or more, preferably all of, IL-5, CXCL1, IP-10, RANTES, MIP-1α, MIP-1β, MIP-2, GM-CSF and/or TNFα. In one embodiment, the compositions of the invention are for use as a vaccine adjuvant. In one embodiment, the compositions of the invention are for use as a vaccine adjuvant in influenza therapy. In certain embodiments, the compositions of the invention are for use in enhancing an immune response against an antigen. In certain embodiments, the invention provides a composition to be administered in combination with an antigen. In certain embodiments, the bacterial strain present in the composition of the invention may be engineered to express an antigen. In certain embodiments, the compositions of the invention are for administration to a patient shortly prior to or after vaccination. Preferably, the invention provides a composition comprising the strain deposited under accession number 42382 at NCIMB, or a derivative or biotype thereof, for any such use as a vaccine adjuvant.

The examples also show that administration of the compositions of the invention can lead to an expansion of a B-cell population. B-cells are known to enhance the immune response to an antigen. Since administration of the compositions of the invention were shown to increase B-cell percentage within the PBMCs, compositions of the invention may be useful as a vaccine adjuvant.

Generally, when used as a vaccine adjuvant, the compositions of the invention will be administered on their own to provide an adjuvant effect for an antigen that has been separately administered to the patient. In certain embodiments, the composition of the invention is administered orally, whilst the antigen is injected parenterally.

In certain embodiments, the bacterial strain of the invention expresses one or more antigens. Generally the antigen will be expressed recombinantly and will be heterologous to the bacterial cell. Therefore, in embodiments of the invention a bacterial strain of the Parabacteroides genus is provided in the composition that expresses a heterologous antigen.

Exemplary antigens, which may be expressed by the bacterial strain of the Parabacteroides genus and/or which may be separately provided in the compositions or administered sequentially or separate to the composition of the invention include: viral antigens, such as viral surface proteins; bacterial antigens, such as protein and/or saccharide antigens; fungal antigens; parasite antigens; and tumor antigens.

The invention is particularly useful for antigens from the following pathogens: influenza virus, HIV, hookworm, hepatitis B virus, herpes simplex virus, rabies, respiratory syncytial virus, cytomegalovirus, Staphylococcus aureus, chlamydia, SARS coronavirus, varicella zoster virus, Streptococcus pneumoniae, Neisseria meningitidis, Mycobacterium tuberculosis, Bacillus anthracis, Epstein Barr virus, human papillomavirus.

Further antigens include glycoprotein and lipoglycan antigens, archaea antigens, melanoma antigen E (MAGE), Carcinoembryonic antigen (CEA), MUC-1, HER2, sialyl-Tn (STn), human telomerase reverse transcriptase (hTERT), Wilms tumour gene (WT1), CA-125, prostate-specific antigen (PSA), Epstein-Barr virus antigens, neoantigens, oncoproteins, amyloid-beta, Tau, PCSK9 and habit forming substances, for example nicotine, alcohol or opiates.

The invention also provides the use of: (i) an aqueous preparation of an antigen (e.g. one or more of those identified above); and (ii) a composition comprising a bacterial strain of the genus Parabacteroides, in the manufacture of a medicament for use as a vaccine adjuvant. Preferably, the bacterial strain is the strain deposited under accession number 42382 at NCIMB, or a derivative or biotype thereof.

The immune response raised by these methods and uses will generally include an antibody response, preferably a protective antibody response.

As used herein, “enhancing” the efficacy of a vaccine, or a subject's immune response, refers to a vaccine of the invention eliciting a greater immune response (such as a humoral immune response) in a subject, when compared to the immune response in a subject who receives the same antigen(s) without the addition of a bacterial strain of the genus Parabacteroides.

### Cell Therapies

### Chimeric Antigen Receptor T Cell (CAR-T) Therapy

Therefore, compositions of the invention may be useful in cell therapy, in particular CAR-T cell therapy. In one embodiment, the compositions of the invention are for use in cell therapy. In one embodiment, the compositions of the invention are for use in CAR-T cell therapy. In one embodiment, compositions of the invention are for use in the therapy of cancer, by enhancing CAR-T. In one preferred embodiment, compositions of the invention are for use in the treatment of chronic lymphocytic leukaemia by enhancing CAR-T. Preferably, the invention provides a composition comprising the strain deposited under accession number 42382 at NCIMB, or a derivative or biotype thereof, for any such use.

In certain embodiments, the compositions of the invention are administered to a patient before T cell adoptive transfer during CAR-T therapy.

In certain embodiments, the compositions of the invention are administered to a patient after T cell adoptive transfer during CAR-T therapy.

Therefore, the compositions of the invention may be useful in cell therapy, in particular in enhancing the response to a cell therapy.

As used herein, “enhancing” the efficacy of a cell therapy, such as CAR-T, refers to a composition of the invention eliciting a greater therapeutic effect from the cell therapy (such as, in the case of CAR-T, a T cell-mediated immune response, in particular against a tumour antigen) in a subject as a result of its administration, when compared to the absence of its administration. For example, a subject treated with a composition of the invention and a cell therapy may exhibit a greater such therapeutic effect from the cell therapy, when compared to a control subject treated with the cell therapy but not the composition of the invention.

### Mesenchymal Stem Cell (MSC) Therapy

Mesenchymal stem cell (MSC) therapy has been reported to have immunostimulatory properties. When MSCs are treated with LPS, they upregulate pro-inflammatory cytokine IL-8 which causes increased B cell proliferation [24]. Therefore, since compositions of the invention were shown to increase B cell proliferation, they may be useful in combination with MSC cell therapy.

### Stem Cell Transplantation Therapy

It has been reported that, instead of using undifferentiated stem cells in stem cell transplantation therapy, it may be beneficial to differentiate stem cells to some extent prior to transplantation. For example, Heng et al. [25] reported that cardiomyogenic differentiation of stem cells may be beneficial by having a higher engraftment efficiency, enhanced regeneration of myocytes and increased restoration of heart function. Also, studies have shown that GI colonisation with certain commensal strains of bacteria can improve survival following allogeneic haematopoietic cell transplant [26]. Since administration of the compositions of the invention stimulated cells, compositions of the invention may be useful for stem cell differentiation in stem cell transplantation therapy. In particular, compositions of the invention are for use in a method of haematopoietic cell transplantation, such as allogeneic haematopoietic cell transplantation.

### Immunosenescence

Fulop et al. [27] identified that a decrease in B cell number are associated with aging in the adaptive immune system. Therefore, compositions of the invention may be used to prevent or delay immunosenescence. In one embodiment, compositions of the invention are for use in preventing immunosenescence. In another embodiment, compositions of the invention are for use in delaying immunosenescence characterised by a decrease in B cell number (B cell immunosenescence). In one embodiment, compositions of the invention are for use in delaying immunosenescence by increasing B cell number. In one embodiment, compositions of the invention are for use in treating diseases caused by immunosenescence. In one embodiment, compositions of the invention are for use in treating aging-related diseases by delaying and/or preventing immunosenescence. Preferably, the invention provides a composition comprising the strain deposited under accession number 42382 at NCIMB, or a derivative or biotype thereof, for any such use.

Furthermore, it has been proposed that vaccine adjuvants may overcome immunosenescence [28]. Since the compositions of the invention are suitable for use as a vaccine adjuvant, compositions of the invention may be useful for preventing or delaying immunosenescence. In another embodiment, compositions of the invention are for use in delaying and/or preventing immunosenescence as a vaccine adjuvant. In another embodiment, compositions of the invention are for use as a vaccine adjuvant, wherein the compositions delay and/or prevent immunosenescence.

Diseases that are associated with immunosenescence include cardiovascular disease, neurodegenerative diseases, such as Alzheimer's disease and Parkinson's disease, cancer, diabetes mellitus type 2 [29] and autoimmune disorders [30].

### Patient Subgroups

As shown in the examples, numerous Parabacteroides strains elicit immunostimulatory effects, such as splenocyte proliferation and cytokine secretion. Accordingly, in any of the therapeutic uses detailed above, compositions of the invention may be particularly effective in immunocompromised or immunosuppressed subjects. The subject may be immunocompromised or immunosuppressed for any reason including, but not limited to, organ recipiency, iatrogenic immunosuppression, the presence of an immunosuppressive infection (such as an HIV infection), and/or tumour-induced immunosuppression. Preferably, the subject has cancer, and is immunocompromised or immunosuppressed as a result of tumour-induced immunosuppression.

Subjects that are immunocompromised or immunosuppressed (e.g., as a result of tumour-induced immunosuppression) may exhibit elevated numbers of regulatory T cells (Tregs) within the lymph nodes and/or within a volume of peripheral blood mononuclear cells (PBMCs), compared to subjects free of disease, in particular subjects free of cancer (see, e.g. [31], [32]). Accordingly, in any of the therapeutic uses detailed above, compositions of the invention are preferably for use in a subject having an elevated number of regulatory T cells (Tregs) within a lymph node, compared to a lymph node of a subject free of disease. More preferably, the subject has cancer, and compositions of the invention are for use in a subject having an elevated number of regulatory T cells (Tregs) within a lymph node (such as a metastatic lymph node), compared to a lymph node of a subject free of cancer. In addition or alternatively, in any of the therapeutic uses detailed above, compositions of the invention are preferably for use in a subject having an elevated number of Tregs within a volume of PBMCs, compared to the same volume of PBMCs from a subject free of disease. More preferably, the subject has cancer, and compositions of the invention are for use in a subject having an elevated number of Tregs within a volume of PBMCs, compared to the same volume of PBMCs from a subject free of cancer. In these embodiments, Tregs may alternatively be defined as CD4+CD25+ cells, or FOXP3+ cells, or CD4+CD25+ and Foxp3+ cells (see [32]) Immunocompromised or immunosuppressed subjects may also exhibit a higher number of myeloid dendritic cells (mDCs) and/or plasmacytoid dendritic cells (pDCs), compared to subjects free of disease, in particular free of cancer (see, e.g. [33]). Accordingly, in addition or alternatively, in any of the therapeutic uses detailed above, compositions of the invention are preferably for use in a subject having an elevated number of mDCs within a volume of PBMCs, compared to the same volume of PBMCs from a subject free of disease. More preferably, the subject has cancer, and compositions of the invention are for use in a subject having an elevated number of mDCs within a volume of PBMCs, compared to the same volume of PBMCs from a subject free of cancer. In addition or alternatively, in any of the therapeutic uses detailed above, compositions of the invention are preferably for use in a subject having an elevated number of pDCs within a volume of PBMCs, compared to the same volume of PBMCs from a subject free of disease. More preferably, the subject has cancer, and compositions of the invention are for use in a subject having an elevated number of pDCs within a volume of PBMCs, compared to the same volume of PBMCs from a subject free of cancer. In these embodiments, pDCs may alternatively be defined as CD11c+ cells, and/or mDCs may alternatively be defined as CD123+ cells (see [33]). Cell numbers and the expression of cell surface markers may be determined using standard methods available in the art, such as flow cytometry (see e.g. [32]).

Modes of Administration Preferably, the compositions of the invention are to be administered to the gastrointestinal tract in order to enable delivery to and/or partial or total colonisation of the intestine with the bacterial strain of the invention. Generally, the compositions of the invention are administered orally, but they may be administered rectally, intranasally, or via buccal or sublingual routes.

In certain embodiments, the compositions of the invention may be administered as a foam, as a spray or a gel.

In certain embodiments, the compositions of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In certain embodiments, the composition of the invention is administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum and other suitable access ports.

The compositions of the invention may be administered once, or they may be administered sequentially as part of a treatment regimen. In certain embodiments, the compositions of the invention are to be administered daily.

In certain embodiments of the invention, treatment according to the invention is accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful and efficacy is observed.

In certain embodiments, the composition of the invention may be administered to a pregnant animal, for example a mammal such as a human in order to reduce the likelihood of disease developing in her child in utero and/or after it is born.

The compositions of the invention may be administered to a patient that has been identified as having a decrease in B-cell number.

The compositions of the invention may be administered as a food product, such as a nutritional supplement.

Generally, the compositions of the invention are for the treatment of humans, although they may be used to treat animals including monogastric mammals such as poultry, pigs, cats, dogs, horses or rabbits. The compositions of the invention may be useful for enhancing the growth and performance of animals. If administered to animals, oral gavage may be used.

### Compositions

The composition of the invention comprises bacteria. In preferred embodiments of the invention, the composition is formulated in freeze-dried form. For example, the composition of the invention may comprise granules or gelatin capsules, for example hard gelatin capsules, comprising a bacterial strain of the invention.

Preferably, the composition of the invention comprises lyophilised bacteria. Lyophilisation of bacteria is a well-established procedure and relevant guidance is available in, for example, references [34,36].

Alternatively, the composition of the invention may comprise a live, active bacterial culture.

In preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [37] and [38].

The composition may be administered orally and may be in the form of a tablet, capsule or powder. Encapsulated products are preferred because organisms from the genus Parabacteroides are anaerobes. Other ingredients (such as vitamin C, for example), may be included as oxygen scavengers and prebiotic substrates to improve the delivery and/or partial or total colonisation and survival in vivo. Alternatively, the probiotic composition of the invention may be administered orally as a food or nutritional product, such as milk or whey based fermented dairy product, or as a pharmaceutical product.

The composition may be formulated as a probiotic.

A composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU; in another example from about 1×10⁷ to about 1×10¹¹ CFU; in another example from about 1×10⁸ to about 1×10¹⁰ CFU; in another example from about 1×10⁸ to about 1×10¹¹ CFU.

In certain embodiments, the dose of the bacteria is at least 10⁹ cells per day, such as at least 10¹⁰, at least 10¹¹, or at least 10¹² cells per day.

In certain embodiments, the composition contains the bacterial strain in an amount of from about 1×10⁶ to about 1×10¹¹ CFU/g, respect to the weight of the composition; for example, from about 1×10⁸ to about 1×10¹⁰ CFU/g. The dose may be, for example, 1 g, 3 g, 5 g, and 10 g.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the pharmaceutical composition comprises 16, 15, 14, 13, 12, 11, 10, 9, 8, 7, 6, 5 or fewer distinct bacterial species. In certain embodiments, the pharmaceutical composition comprises 4 or fewer distinct bacterial species. In certain embodiments, the pharmaceutical composition comprises 3 or fewer distinct bacterial species. In certain embodiments, the pharmaceutical composition comprises 2 or fewer distinct bacterial species. In certain embodiments, the pharmaceutical composition comprises Parabacteroides distasonis, merdae, johnsonii or goldsteinii and no other bacterial species. In preferred embodiments, the compositions of the invention comprise a single strain of Parabacteroides distasonis, merdae, johnsonii or goldsteinii and no other bacterial strains or species. Such compositions may comprise only de minimis or biologically irrelevant amounts of other bacterial strains or species. Strikingly, the examples demonstrate that compositions comprising only a single strain of the invention can have potent effects, with no reliance on co-administration with other strains or species.

In certain embodiments, the invention provides the above pharmaceutical composition, wherein the composition is administered at a dose of between 500 mg and 1000 mg, between 600 mg and 900 mg, between 700 mg and 800 mg, between 500 mg and 750 mg or between 750 mg and 1000 mg. In certain embodiments, the invention provides the above pharmaceutical composition, wherein the lyophilised bacteria in the pharmaceutical composition is administered at a dose of between 500 mg and 1000 mg, between 600 mg and 900 mg, between 700 mg and 800 mg, between 500 mg and 750 mg or between 750 mg and 1000 mg.

Typically, a probiotic, such as the composition of the invention, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

In certain embodiments, the probiotic composition of the present invention includes a prebiotic compound in an amount of from about 1 to about 30% by weight, respect to the total weight composition, (e.g. from 5 to 20% by weight). Carbohydrates may be selected from the group consisting of: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

The compositions of the invention may comprise pharmaceutically acceptable excipients or carriers. Examples of such suitable excipients may be found in the reference [39]. Acceptable carriers or diluents for therapeutic use are well known in the pharmaceutical art and are described, for example, in reference [40]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavoured milks, ice cream; milk-containing food such as sweets.

In certain embodiments, the compositions of the invention contain a single bacterial strain or species and do not contain any other bacterial strains or species. Such compositions may comprise only de minimis or biologically irrelevant amounts of other bacterial strains or species. Such compositions may be a culture that is substantially free from other species of organism.

The compositions for use in accordance with the invention may or may not require marketing approval.

In some cases, the lyophilised bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain of the invention; and a pharmaceutically acceptable excipient, carrier or diluent; wherein the bacterial strain is in an amount sufficient to treat a disorder when administered to a subject in need thereof.

In certain embodiments, the invention provides a pharmaceutical composition comprising: a bacterial strain of the invention; and a pharmaceutically acceptable excipient, carrier or diluent; wherein the bacterial strain is in an amount sufficient to treat or prevent a disease or condition.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent a disease or condition mediated by a reduced immune response.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the invention provides the above pharmaceutical composition, wherein the composition is administered at a dose of 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of p-hydroxybenzoic acid.

In certain embodiments, the invention provides the above pharmaceutical composition, wherein said bacterial strain is lyophilised.

In certain embodiments, the invention provides the above pharmaceutical composition, wherein when the composition is stored in a sealed container at about 4.0 or about 25.0 and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

### Culturing Methods

The bacterial strains for use in the present invention can be cultured using standard microbiology techniques as detailed in, for example, references [41-43].

The solid or liquid medium used for culture may be YCFA agar or YCFA medium. YCFA medium may include (per 100 ml, approximate values): Casitone (1.0 g), yeast extract (0.25 g), NaHCO₃ (0.4 g), cysteine (0.1 g), K₂HPO₄ (0.045 g), KH₂PO₄ (0.045 g), NaCl (0.09 g), (NH₄)₂SO₄ (0.09 g), MgSO₄.7H₂O (0.009 g), CaCl₂ (0.009 g), resazurin (0.1 mg), hemin (1 mg), biotin (1 μg), cobalamin (1 μg), p-aminobenzoic acid (3 μg), folic acid (5 μg), and pyridoxamine (15 μg). YCFA+medium has the following composition:

| | | |
|---|---|---|
| Bacto casitione | 1.0 | g |
| Yeast extract | 0.25 | g |
| Sodium hydrogen carbonate | 0.4 | g |
| Glucose | 0.2 | g |
| Cellobiose | 0.2 | g |
| Soluble starch | 0.2 | g |
| Mineral solution 1 | 15 | ml |
| Mineral solution 2 | 15 | ml |
| SCFA solution | 0.31 | ml |
| Haemin solution | 1 | ml |
| Vitamin solution 1 | 100 | μl |
| Vitamin solution 2 | 100 | μl |
| Resazurin solution | 0.1 | ml |
| Cysteine | 0.1 | g |
| d. H2O to a total volume of: | 100 | m |

Mineral solution 1: K₂HPO₄-3.0 g; d.H₂O to a total volume of 11
Mineral solution 2: KH₂PO₄-3.0 g; (NH₄)₂SO₄_6.0 g; NaCl-6.0 g; MgSO₄-0.6 g; CaCl₂-0.6 g; d. H₂O to a total volume of 11
Resazurin solution: 0.1% powdered resazurin in 100 ml distilled water.
Short chain fatty acid solution: Acetic acid—17 ml; Propionic acid-6 ml; n-Valeric acid-1 ml; Iso-Valeric acid-1 ml; Iso-Butyric acid-1 ml
Haemin solution: KOH-0.28 g Ethanol 95%-25 ml; Haemin-100 mg; d. H₂O to a total volume of 100 ml
Vitamin solution 1: Biotin-1 mg; Cobalamin-1 mg; p-Aminobenzoic acid-3 mg; Folic acid-5 mg; Pyridoxamine-15 mg; d. H₂O to a total volume of 100 ml
Vitamin solution 2: Thiamine-5 mg; Riboflavin-5 mg; d. H₂O to a total volume of 100 ml

### Vaccine Compositions

The inventors have identified that the bacterial strains of the invention are useful as vaccine adjuvants. Therefore, the bacterial strains of the invention may also be useful for preventing diseases or conditions, when administered in vaccine compositions as the adjuvant, in combination with one or more antigens, such as pathogen antigens or tumour antigens. Accordingly, the invention also provides a vaccine composition comprising a bacterial strain of the genus Parabacteroides (as defined above), and one or more antigens, such as pathogen antigens or tumour antigens. Pathogen antigens include viral antigens, such as viral surface proteins; bacterial antigens, such as protein and/or saccharide antigens; fungal antigens; and parasite antigens.

Antigens in vaccine compositions of the invention include those from the following pathogens:

influenza virus, HIV, hookworm, hepatitis B virus, herpes simplex virus, rabies, respiratory syncytial virus, cytomegalovirus, Staphylococcus aureus, chlamydia, SARS coronavirus, varicella zoster virus, Streptococcus pneumoniae, Neisseria meningitidis, Mycobacterium tuberculosis, Bacillus anthracis, Epstein Barr virus, human papillomavirus. Influenza virus antigens are preferred.

Further antigens in vaccine compositions of the invention include glycoprotein and lipoglycan antigens, archaea antigens, melanoma antigen E (MAGE), Carcinoembryonic antigen (CEA), MUC-1, HER2, sialyl-Tn (STn), human telomerase reverse transcriptase (hTERT), Wilms tumour gene (WT1), CA-125, prostate-specific antigen (PSA), neoantigens, oncoproteins, amyloid-beta, Tau, PCSK9 and habit forming substances, for example nicotine, alcohol or opiates.

In some embodiments, the vaccine composition comprises a pharmaceutically acceptable excipient or carrier. In some embodiments, the vaccine composition comprises further adjuvants, such aluminium salts (in particular aluminium hydroxide, aluminium phosphate or aluminium sulphate). In other embodiments the vaccine composition does not comprise a further adjuvant (that is, the bacterial strain according to the invention is the only adjuvant in the composition).

In some embodiments, the bacterial strain of the genus Parabacteroides (as defined above) expresses the one or more antigens in the vaccine composition. Generally the antigen will be expressed recombinantly and will be heterologous to the bacterial cell. Therefore, in some embodiments, the bacterial strain of the genus Parabacteroides (as defined above) is provided in the vaccine composition that expresses a heterologous antigen.

In certain such embodiments, the bacterial strains of the invention may be killed, inactivated or attenuated. In certain embodiments, the vaccine compositions are for administration via injection, such as via subcutaneous injection.

Vaccine compositions of the invention may further comprise the composition features as defined above (see “Compositions” section).

Vaccine compositions of the invention are also for use in medicine, including any of the therapeutic uses defined above.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references [44] and [45,51], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

The word “substantially” does not exclude “completely” e.g. a composition which is “substantially free” from Y may be completely free from Y. Where necessary, the word “substantially” may be omitted from the definition of the invention.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref. [52]. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref [53].

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### MODES FOR CARRYING OUT THE INVENTION

### Example 1—Basic Cell Phenotyping on PBMCs from Healthy Donors

### Bacterial Strain

Parabacteroides distasonis strain NCIMB 42382

### Method

### PBMCs Treatment

Frozen healthy human PBMCs were purchased from Stem Cells Technologies (Cambridge UK). Briefly cells were thawed and left to rest overnight in full growth media (RPMI 1640 with 10% FBS, 2 mM L. Glutamine and 100 U/ml penicillin, 100 μg/ml streptomycin) in CO2 incubator at 37° C. For the experiment cells were plated at a density of 750,000 Cell/well in 48 well plates and treated in full growth media with 10% bacteria supernatants in the presence or absence of 1 ng/ml LPS. Cell culture media was added to untreated wells. Cells were left to rest for 72 h, thereafter cell free supernatants were collected and spun down for 3 minutes at 10,000 g at 4° C. Samples were stored at −80° C. for cytokine analysis.

### Immunophenotyping

1.5×10⁶ cells per sample were stained with viability fixable dye (Miltenyi) to discriminate between live and dead cells for 10 min at RT. Afterwards the cells were stained with the cocktail of antibodies listed below (Miltenyi) for basic immunophenotyping (CD3/CD4/CD8/CD25/CD127 and CD19) and incubated for 10 min at RT.

Experiments were carried out to measure the percentage of the following cell populations:
CD4+CD3+ cells (markers of CD4 T-helper cells)
CD4+CD25+ cells (markers of CD4+ activated cells)
CD25++CD17− cells out of the CD4+ cell population (markers of Tregs cells)
CD8+CD3+ cells (markers of cytotoxic T cells)
CD25+CD8+ cells (markers of CD8+ activated cells)
CD19+CD3− cells (markers of B cells).

The ratio of CD8+/Tregs and the ratio of activated CD8/Treg cells were determined.

### Antibodies

| | |
|---|---|
| Aria | AB-Fluorochrome |
| V2 | CD3-VioBlue |
| APC | CD4-APC-Vio 770 |
| Cy7 | |
| PE-Cy7 | CD8-PE-Vio 770 |
| PE | CD25-PE |
| APC | CD127-APC |
| FITC | CD19-VioBright 515 |

### Results

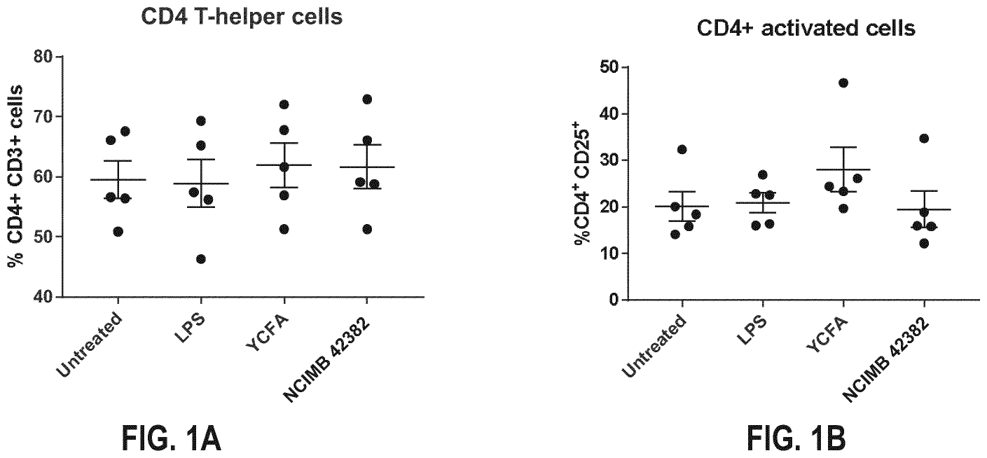

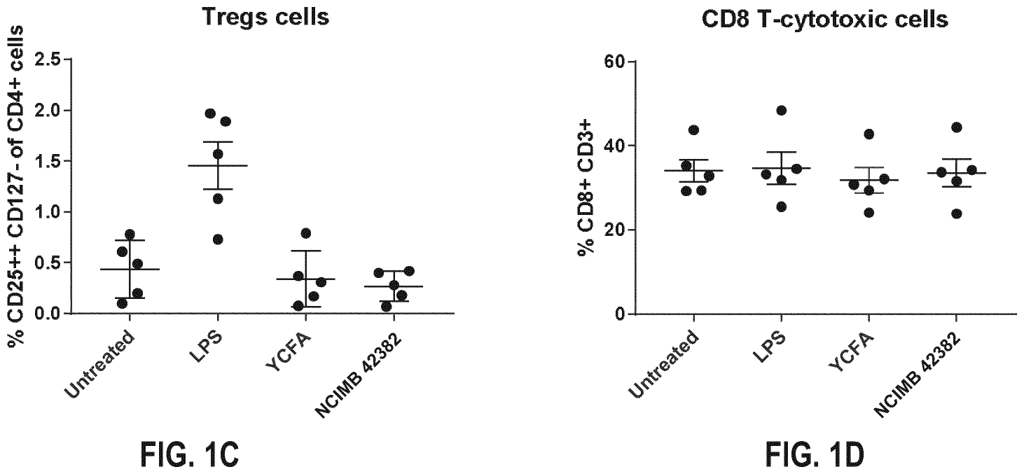

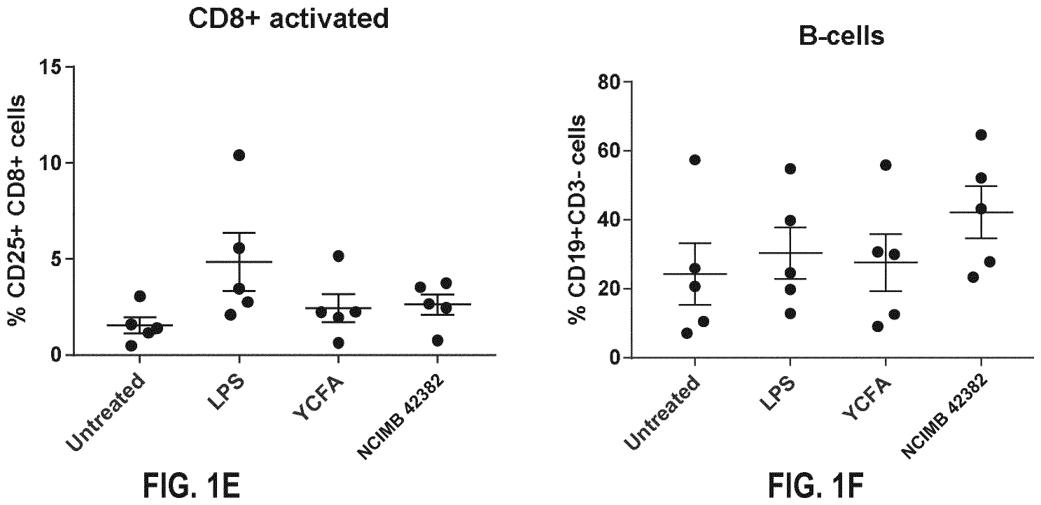

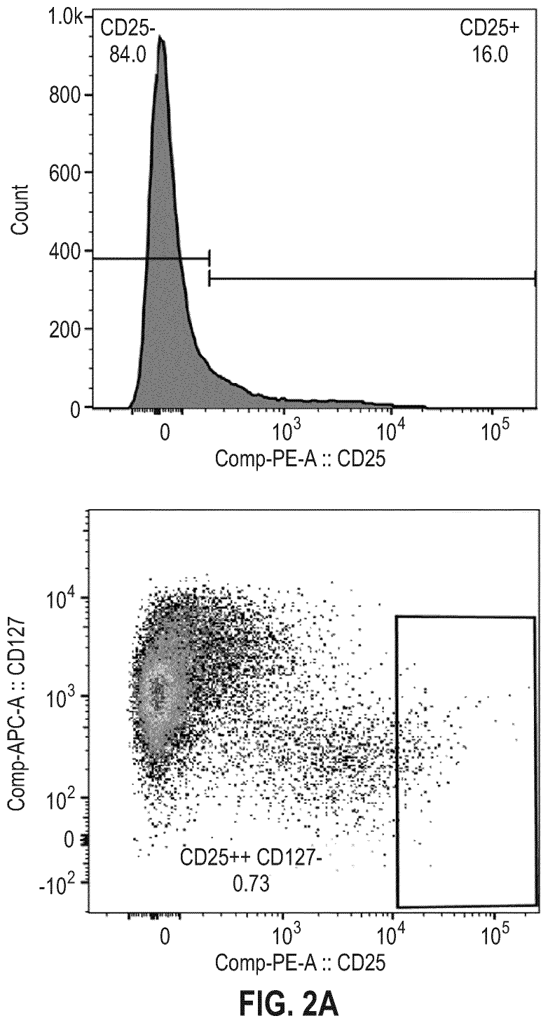

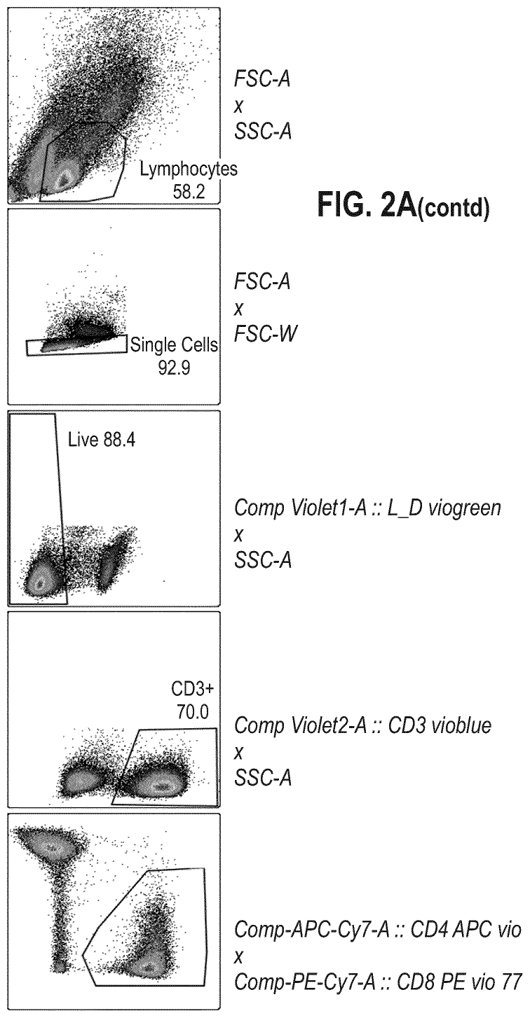

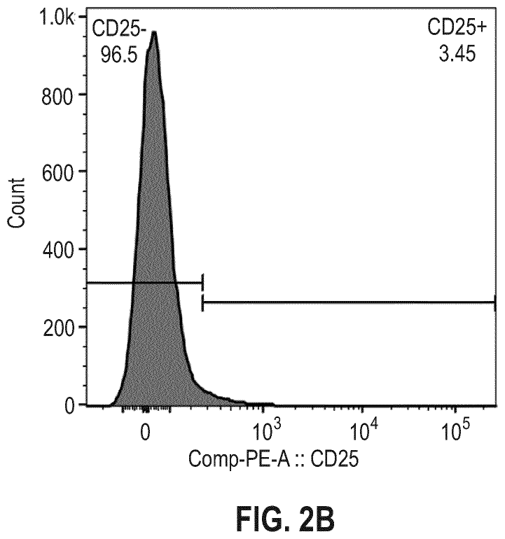

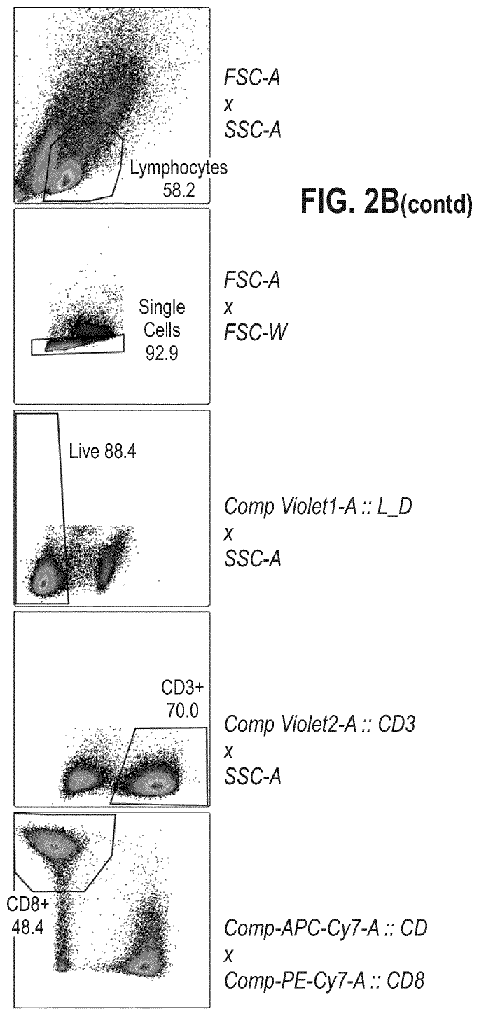

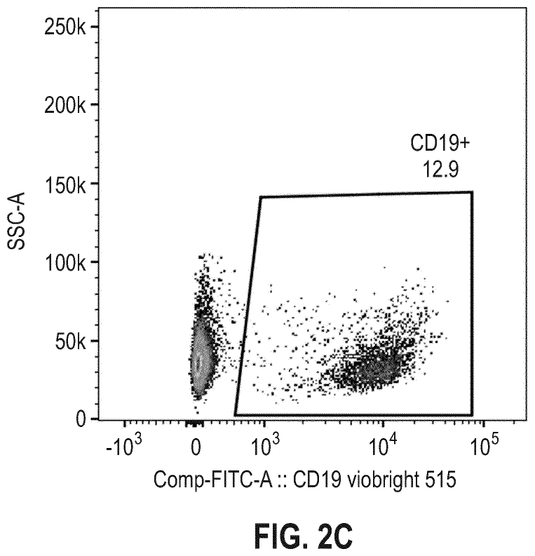

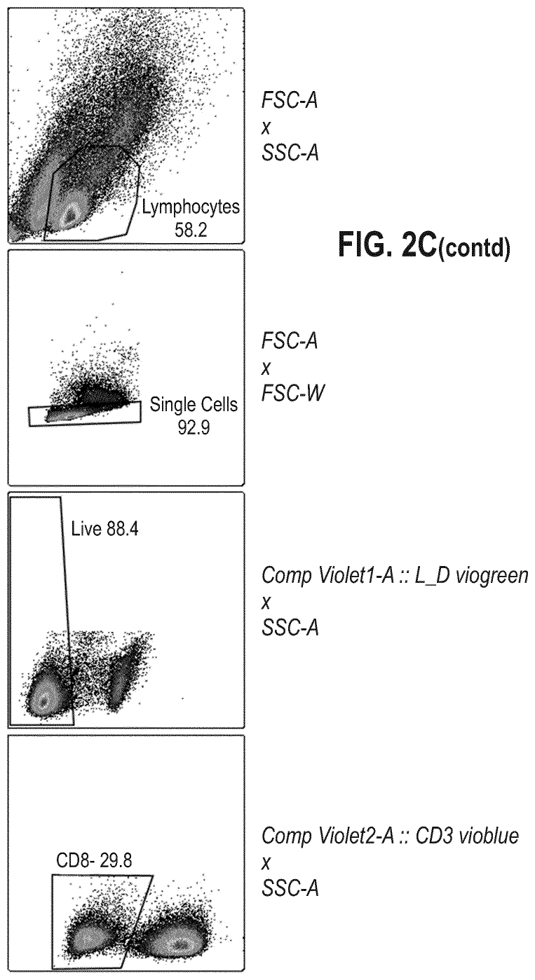

The results of the experiments are shown in FIG. 1.

The most surprising result is the effect of NCIMB 42382 treatment on the percentage of CD19+CD3-cells, which represent B cells (see FIG. 1F). NCIMB 42382 selectively increased the percentage of B-cells in the PBMC population. NCIMB 42382 treatment did not significantly change the percentage of CD4 T-helper cells, CD4+ activated cells, Treg cells, cytotoxic T cells or CD8+ activated cells.

### Discussion

The observation that treatment with NCIMB 42382 selectively increased the percentage of B cells is supportive of the efficacy of strains from the Parabacteroides genus as vaccine adjuvants as well as being effective in the treatment of other conditions characterised by decreases in B-cell levels such as immunosenescence.

### Example 2—Cytokine Analysis of PBMCs from Healthy Donors

### Introduction

The inventors sought to further analyse PBMCs post-incubation with NCIMB 42382. The inventors analysed the expression of particular cytokines from PBMCs known to be associated with vaccine adjuvant efficacy, namely MCP-1.

### Bacterial Strain

Parabacteroides distasonis strain NCIMB 42382

### Method

### PBMCs Treatment

PBMCs were treated as described in Example 1.

### Cytokine Quantification

Cytokine quantification was conducted using a ProcartaPlex multiplex immunoassay following the manufacturer's recommendations (Thermo Fischer Scientific). Briefly, 50 μl of cell-free co-culture supernatants were used for cytokine quantification using a MAGPIX® MILLIPLEX® system (Merck) with the xPONENT software (Luminex, Austin, Tex., USA). Data was analysed using the MILLIPLEX® analyst software (Merck) using a 5-parameter logistic curve and background subtraction to convert mean fluorescence intensity to pg/ml values.

### Results

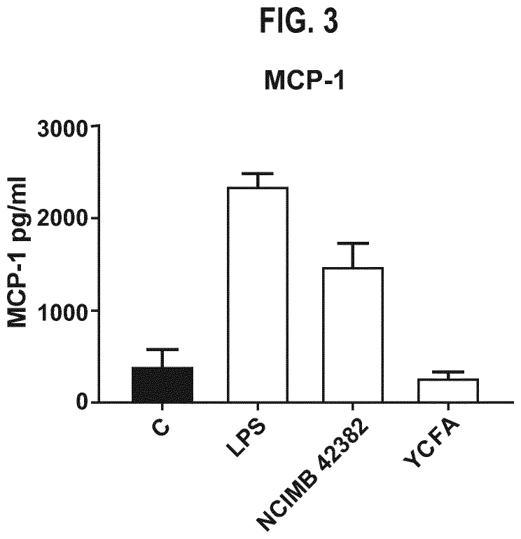

The results are shown in FIG. 3. The results for the Cytokine analysis of NCIMB 42382 in PBMC culture from healthy donors showed an increase in the expression of MCP-1.

### Discussion

This shows that NCIMB 42382 effectively elicits an increase in MCP-1 from PBMCs, a cytokine associated with vaccine adjuvant activity.

### Example 3—Effect of NCIMB 42382 on TNF-Alpha Secretion by the HT29 Cell Line

### Method

Differentiated HT29 cells form polarized apical/mucosal and basolateral/serosal membranes that are impermeable and are structurally and functionally similar to epithelial cells of the small intestine.

HT29 cells were plated in 12 well plates at a density of 200,000 cells/well. Cells were differentiated for 10 days (media change every 2 days). The day of the experiment cells were placed in the anaerobic hood and washed with anaerobic equilibrated HANKs solution. Then 900 μl of growth media (without FBS and antibiotics) was added to the cells. Bacterial cells were resuspended growth media (without FBS and antibiotics) and were then added at 10\hat{ }7 in total in 100 μl. Cells were co-incubated with bacteria for 2 hr in an anaerobic hood. Afterwards cells were washed in growth media without FBS but containing antibiotics. Cells were left to rest in 1 ml of ThP1 condition media for 24 h. After 24 h incubation the supernatant was collected and spun down at 10,000 g for 3 min and 4° C. Samples were frozen at −80° C. until further use.

ThP1 condition media: Thp1 were seeded on T25 flask at density of 4×10\hat{ }6/flask. Cells were treated in RPMI media (contain 2 mM L-glutamine without FBS) with 1 ug/ml LPS or LPS+5 mM ATP (ATP added 3 hours after LPS). Cells were left to rest for 24 hr. Thereafter Condition Media (CM) was collected by spinning down the cells at 250 g for 5 min and RT. Different CMs were used to treat HT29 Cells. A small aliquot was frozen at 80° C. for ELISA.

Supernatants from the different samples were collected and cytokine analysis performed according to manufacturer's instruction using the human TNF-α ELISA kit from Peprotech. GraphPad Prism7 was used to plot and analysed the data.

### Results and Discussion

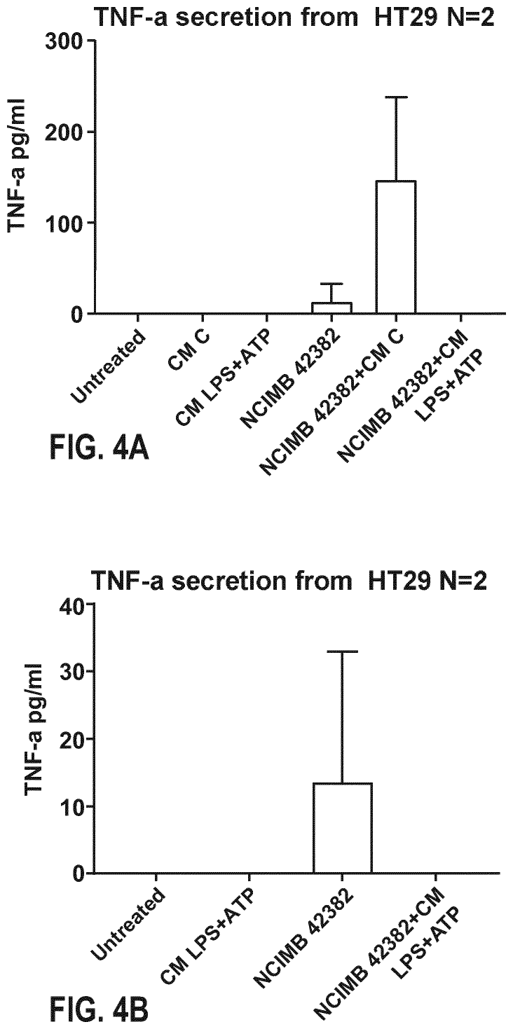

NCIMB 42382 supernatant either alone or with Thp1 conditioned media (CM) induced TNF-α secretion from the HT29 cancer cell line (colorectal cancer)—see FIGS. 4B and 4A respectively. TNF-α is a potent immunostimulatory cytokine, the secretion of which is induced by vaccine adjuvants [54]; moreover TNF-α itself has reported effects as a vaccine adjuvant [55]. These data provide further evidence for NCIMB 42382 having efficacy as a vaccine adjuvant, for example in cancer therapy.

### Example 4—Fermentation Profile of NCIMB 42382

### Method

Rapid ID 32A testing was carried out on NCIMB 42382 colonies as per manufacturer's instructions. A single bead from an NCIMB 42382 bead stock generated on 26 Jun. 2015 was used to inoculate a YCFA agar plate (E&O Labs) which was incubated for 24 hours at 37° C. in an anaerobic workstation. Colonies were removed from the plate and resuspended in a 2 ml ampoule of API® Suspension Medium (bioMerieux), and this suspension was used to inoculate a Rapid ID 32A strip (bioMerieux) as per manufacturer's instructions. The strip was incubated and developed according to manufacturer's instructions, and the colour of each cupule was recorded and assigned a value of negative, intermediate or positive.

API® 50 CHL testing was carried out as per manufacturer's instructions with some slight alterations. A single bead from an NCIMB 42382 glycerol stock generated on 14 Aug. 2015 was used to inoculate an YCFA agar plate (E&O Labs) which was incubated for 24 hours at 37° C. in an anaerobic workstation. A single colony from this plate was used to inoculate a culture in YCFA broth (E&O Labs) and this was incubated for 16-18 hours at 37° C. anaerobically. This culture was diluted tenfold in API® CHL Medium (bioMerieux) to create a suspension that was used to inoculate each cupule on an API® 50 CH test panel (bioMerieux). Test strips were incubated in a humidified incubation box at 37° C. anaerobically for 48 hours, following which the colour of each cupule was recorded and assigned a value of negative, intermediate or positive.

### Results and Discussion

Using Rapid ID 32A analysis, NCIMB 42382 tested positive for fermentation of α-galactosidase, β-galactosidase, α-glucosidase, β-glucosidase, alkaline phosphatase, and utilisation of arginine, leucyl-glycine, leucine, alanine, histidine and glutamyl glutamic acid (FIG. 5A). Using API® 50 CHL, NCIMB 42382 tested positive for utilisation of the following carbohydrate sources: fructose, mannose, mannitol, sorbitol, arbutin, esculin, maltose, lactose, melibiose, sucrose, raffinose, starch, glycogen, turanose and fucose (FIG. 5B). Intermediate reactions were observed for xylose, N-acetylglucosamine, amygdalin, salicin, cellobiose, trehalose, melezitose and gentiobiose. Bacterial strains exhibiting either a highly similar or the same fermentation profile as NCIMB 42382, for carbohydrates and/or amino acids (in particular, carbohydrates), are expected to be useful as vaccine adjuvants, for treating, preventing or delaying immunosenescence, or for use in enhancing a cell therapy.

### Example 5—Effect of Parabacteroides Strains on Splenocyte Proliferation

### Method

Splenocytes were freshly prepared from spleen dissected from female C57BL/6 mice between 6 and 8 weeks of age. Briefly, splenocytes were plated at 900,000 cells/well in 96 well plates in RPMI 1640 with 10% FBS, 2 mM L-Glutamine, 100 U/ml penicillin, 100 μg/ml streptomycin and 55 W of β-mercaptoethanol. Cells were left untreated (resting) or treated with 10% bacterial media YCFA+(blank media) or 10% cell-free bacterial supernatant from stationary culture of various strains and incubated for 72 h in a CO₂ incubator at 37° C. Each Parabacteroides strain was cultured and supernatant prepared as follows: 100 μL of a Research Cell Bank vial was used to inoculate 10 mL of YCFA+broth. The culture was incubated overnight in an anaerobic workstation at 37° C. Each overnight culture was used to inoculate five Hungate tubes containing 10 mL of fresh growth medium with a 10% subculture. Culture tubes were incubated until they reached early stationary phase, following which cell-free supernatants (CFS) were collected as follows. Individual culture tubes were combined and the bacterial density (O.D. 600 nm) was recorded. Cell-free supernatant of the Parabacteroides strain was obtained by centrifugation (5000×g for 15 minutes) and filtration through a 0.45 μm followed by a 0.22 μm filter.

MTT assay kit was purchased from Merck Millipore (Cat n. CT01). After 72 h incubation, 10 μl of MTT solution was added to each well, cells were incubated in a CO₂ incubator for 4 h. Afterwards 100 μl of isopropanol/0.04 M HCL solution was added to each well and the absorbance was measured at 560 nm wavelength with a reference wavelength of 655 nm.

### Results

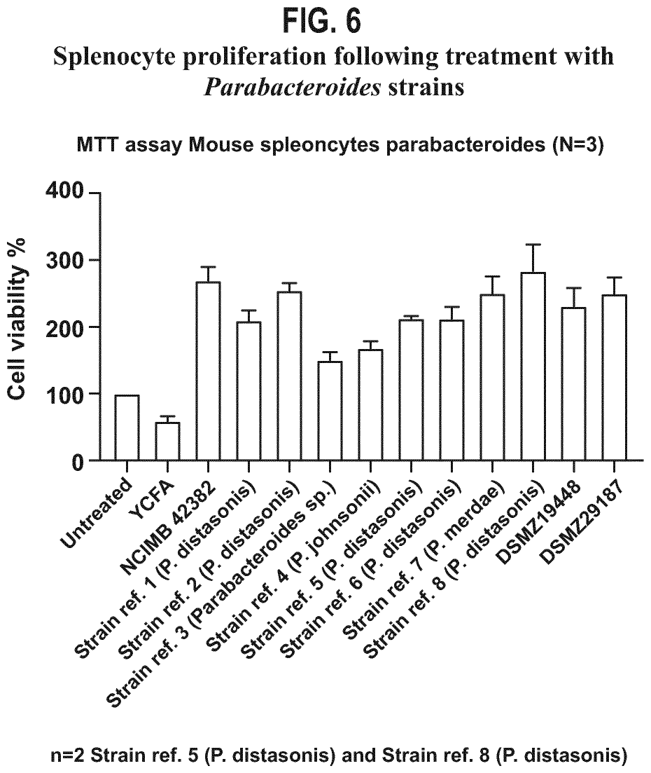

The Parabacteroides strains tested were NCIMB 42382 (P. distasonis), strain ref 1 (P. distasonis), strain ref 2 (P. distasonis), strain ref 3 (Parabacteroides sp.), strain ref 4 (P. johnsonii), strain ref 5 (P. distasonis), strain ref 6 (P. distasonis), strain ref 7 (P. merdae), strain ref 8 (P. distasonis), the strain deposited under accession no. DSMZ19448 (P. goldsteinii), the strain deposited under accession no. DSMZ29187 (P. goldsteinii). All strains induced proliferation of the splenocytes after 72 h culture when compared to YCFA+ or untreated cells (FIG. 6). Splenocytes include various subsets of immune cells such as T cells, B cells and macrophages [56]. Therefore, these data demonstrate that treatment with Parabacteroides strains elicits immunostimulatory effects, indicating that they may act as vaccine adjuvants, therapeutics for treating, preventing or delaying immunosenescence, or adjunct therapeutics for enhancing a cell therapy such as CAR-T.

### Example 6—Effect of Parabacteroides Strains on Cytokine Secretion from Splenocytes

### Method

Splenocytes were prepared and treated with bacterial supernatant as per Example 5. Afterwards the cells were spun down for 5 minutes at 500 g at 4° C. and cell free supernatants were collected, and stored at −80° C. for cytokine analysis. Cytokine quantification was conducted using a 26-plex Mouse ProcartaPlex multiplex immunoassay following the manufacturer's recommendations (Thermo Fischer Scientific). Briefly, 50 μl of cell-free co-culture supernatants were used for cytokine quantification using a MAGPIX® MILLIPLEX® system (Merck) with the xPONENT software (Luminex, Austin, Tex., USA). Data was analysed using the MILLIPLEX® analyst software (Merck) using a 5-parameter logistic curve and background subtraction to convert mean fluorescence intensity to pg/ml values.

### Results

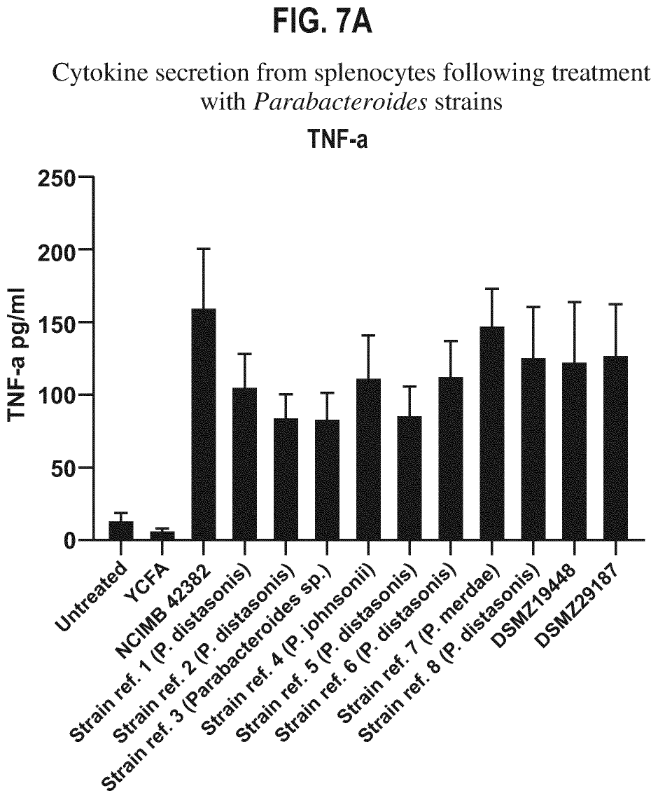

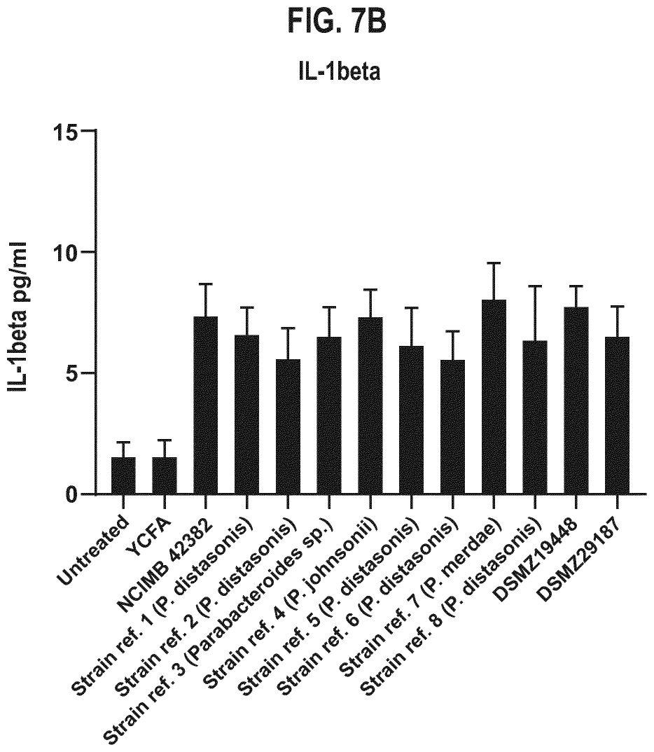

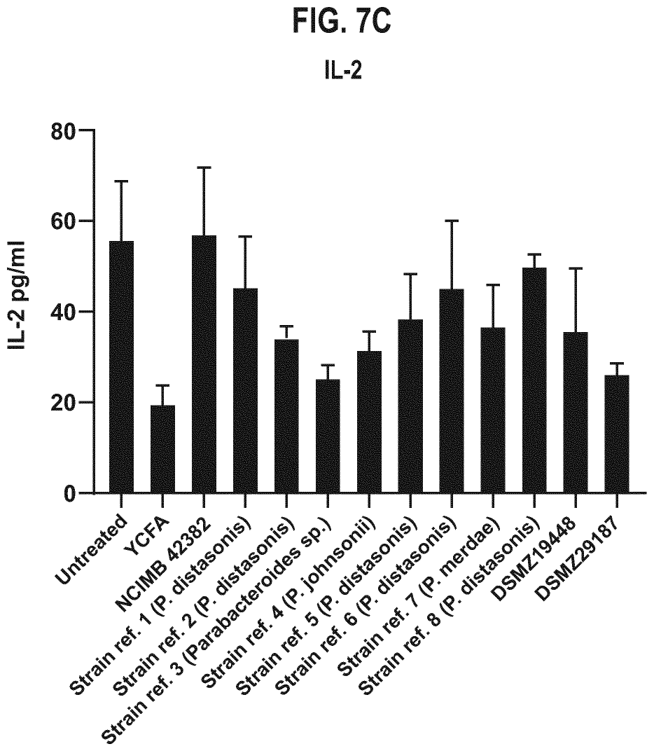

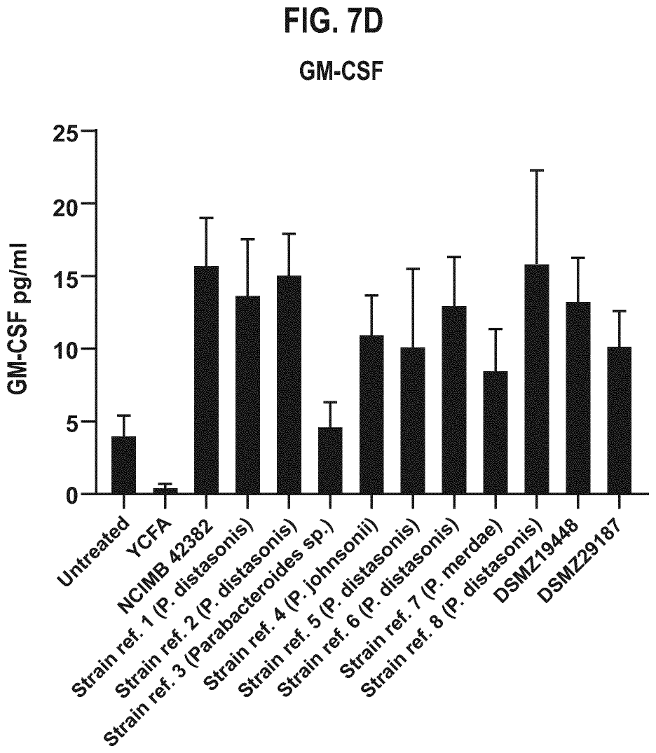

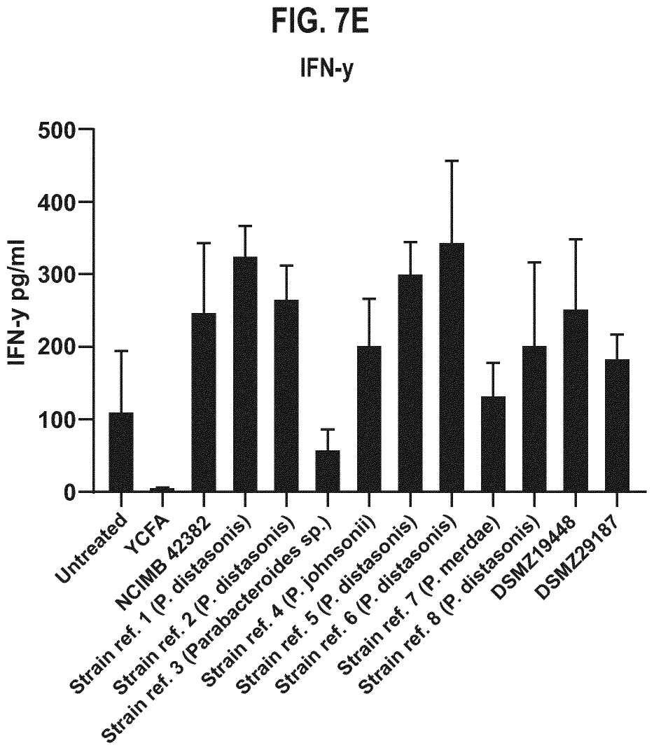

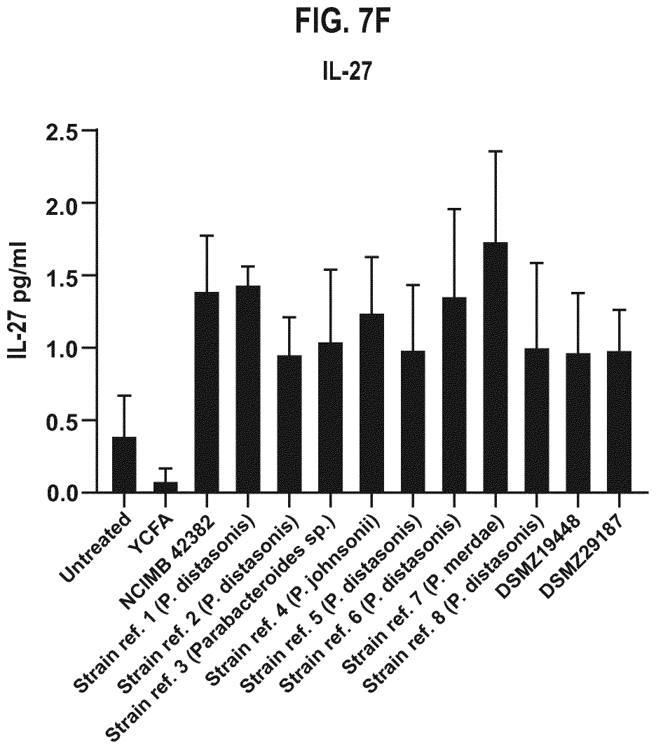

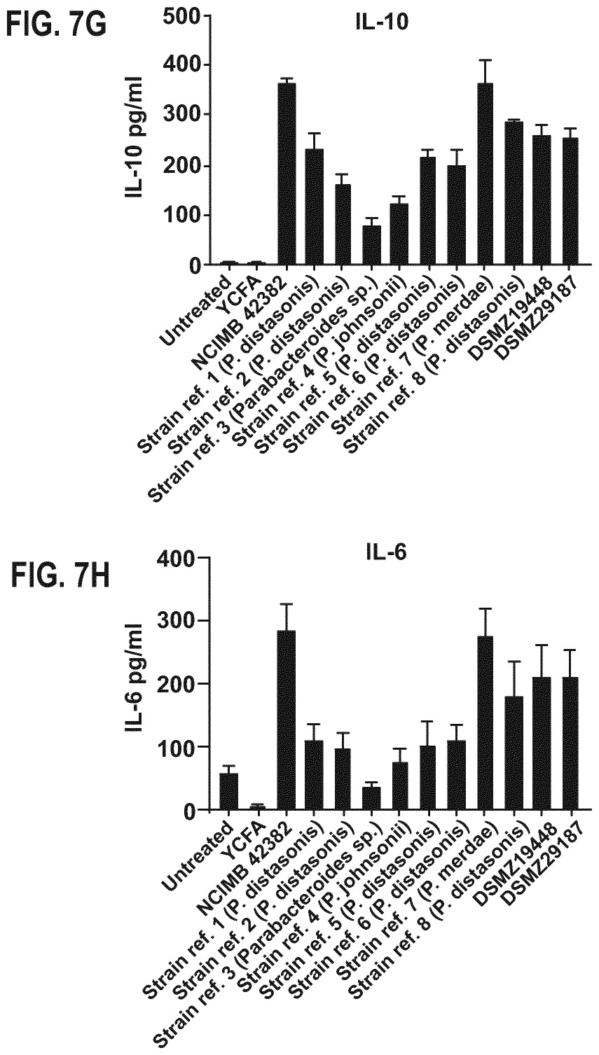

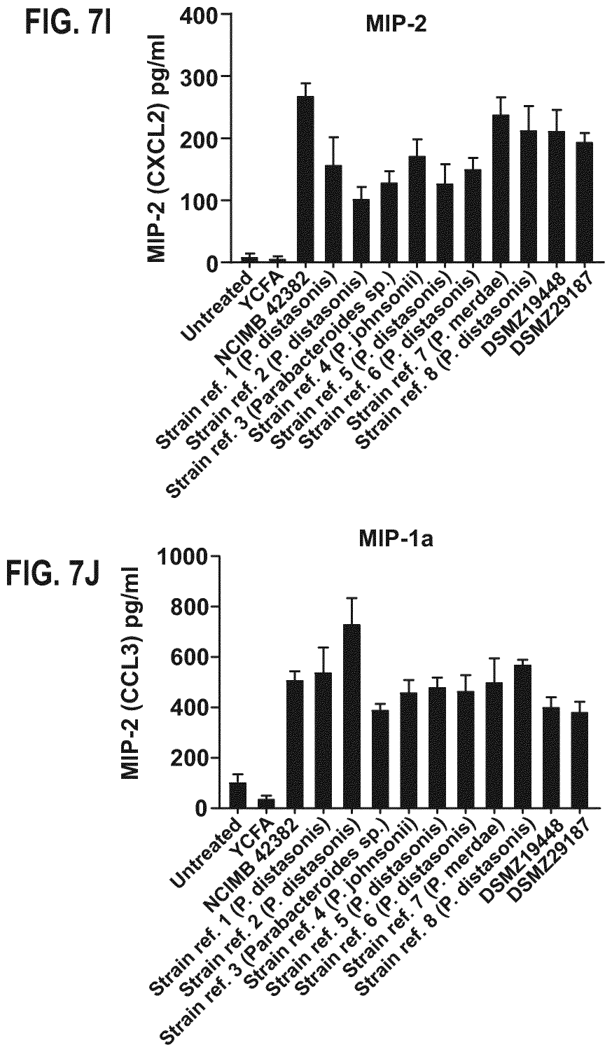

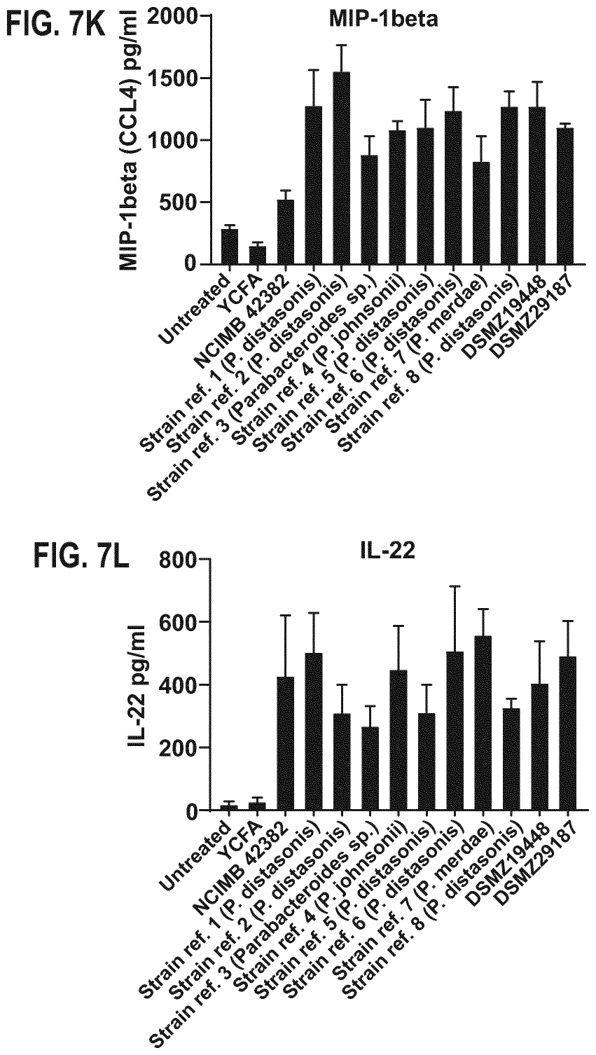

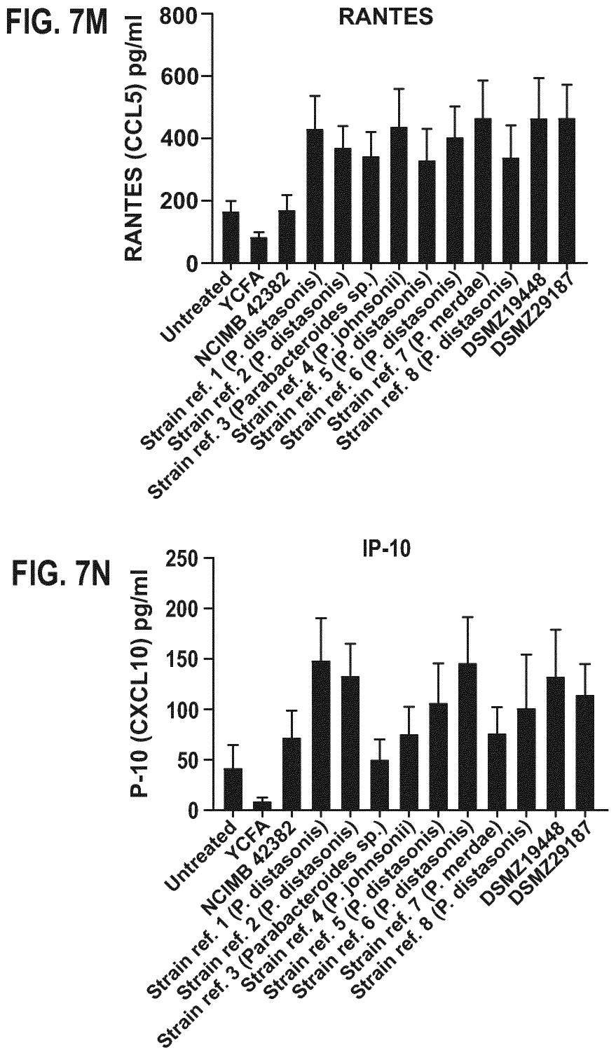

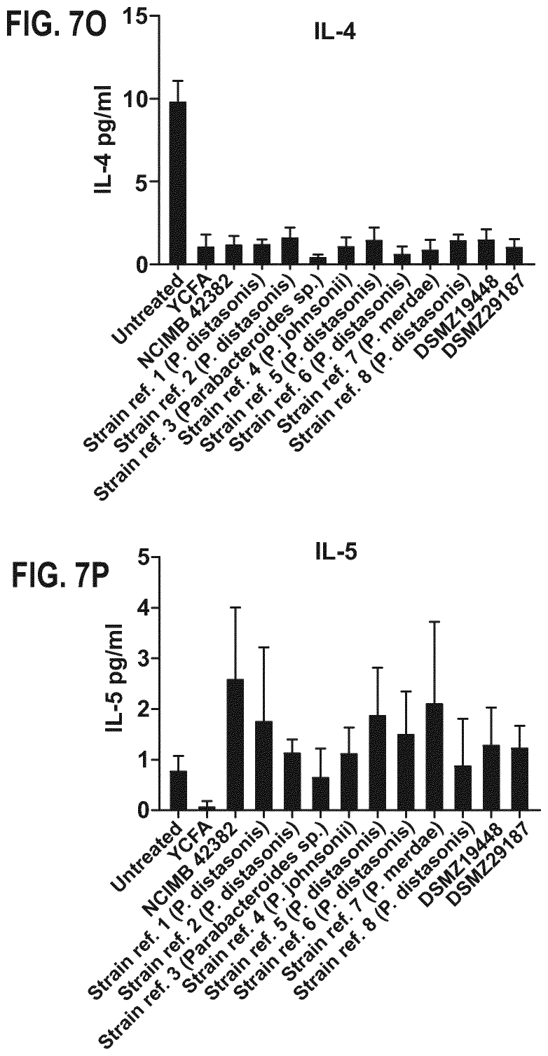

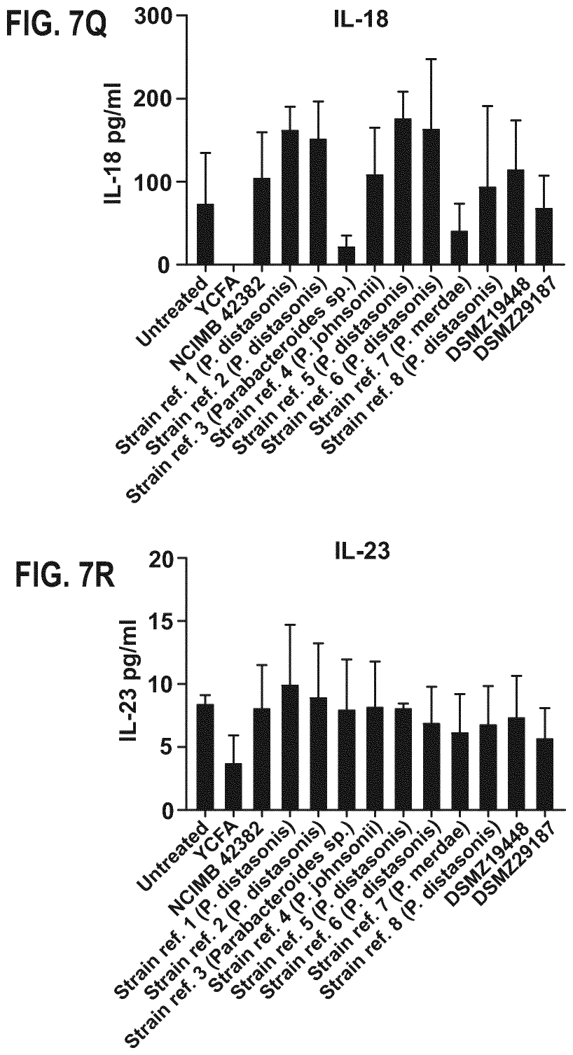

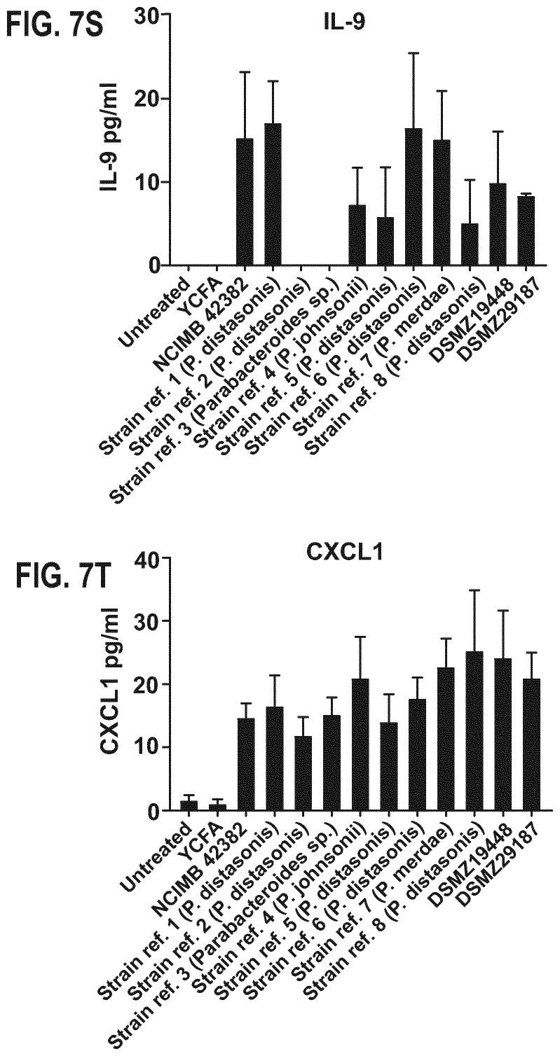

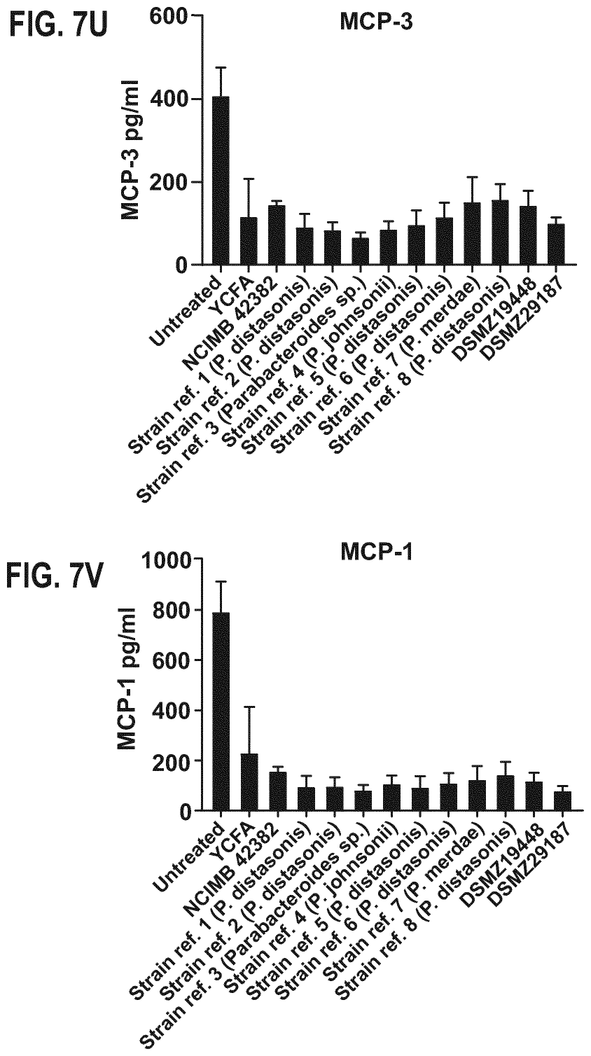

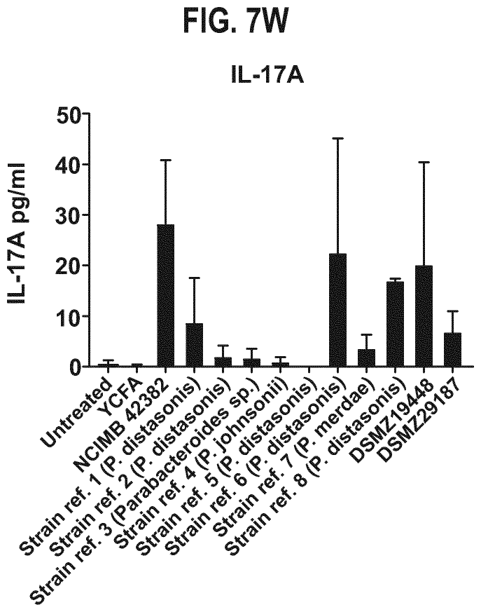

The Parabacteroides strains tested were NCIMB 42382 (P. distasonis), strain ref 1 (P. distasonis), strain ref 2 (P. distasonis), strain ref 3 (Parabacteroides sp.), strain ref 4 (P. johnsonii), strain ref 5 (P. distasonis), strain ref 6 (P. distasonis), strain ref 7 (P. merdae), strain ref 8 (P. distasonis), DSMZ19448 (P. goldsteinii), DSMZ29187 (P. goldsteinii), and the results are shown in FIG. 7. All Parabacteroides strains tested elicited greater secretion of TNF-α, IL-113, IL-27, IL-10, MIP-2, MIP-la, MIP-113, IL-22, IL-5 and CXCL1 than the YCFA+media and untreated controls. Furthermore, all Parabacteroides strains tested elicited greater secretion of IL-2, GM-CSF, IFN-γ, IL-6, IP-10, IL-18, IL-23 and RANTES than the YCFA+media control. Therefore, these data further demonstrate that treatment with Parabacteroides strains elicits immunostimulatory effects, indicating that they may act as vaccine adjuvants, therapeutics for treating, preventing or delaying immunosenescence, or adjunct therapeutics for enhancing a cell therapy such as CAR-T. Many of the upregulated cytokines/chemokines (IL-5, CXCL1, IP-10, RANTES, MIP-1α, MIP-113 and MIP2, for example) can recruit immune cells, and thus may act to create a localised immune response. MF59, an adjuvant already known in the art, is thought to act by this mechanism (inter alia, MIP-1α, MIP-113 and RANTES upregulation) [57]. Furthermore, GM-CSF is known to provide an adjuvant effect for clinically-approved vaccines [58], thereby further indicating utility of Parabacteroides strains as vaccine adjuvants.

### Example 7—Effect of Further Parabacteroides Strains on Cytokine Secretion from Splenocytes

### Method

These experiments were conducted as described in Example 6.

### Results

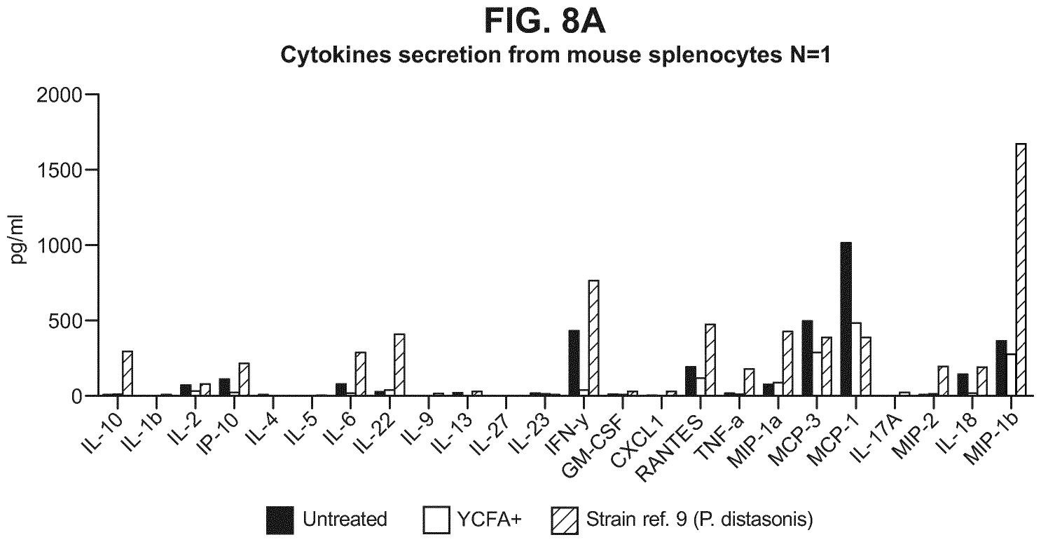

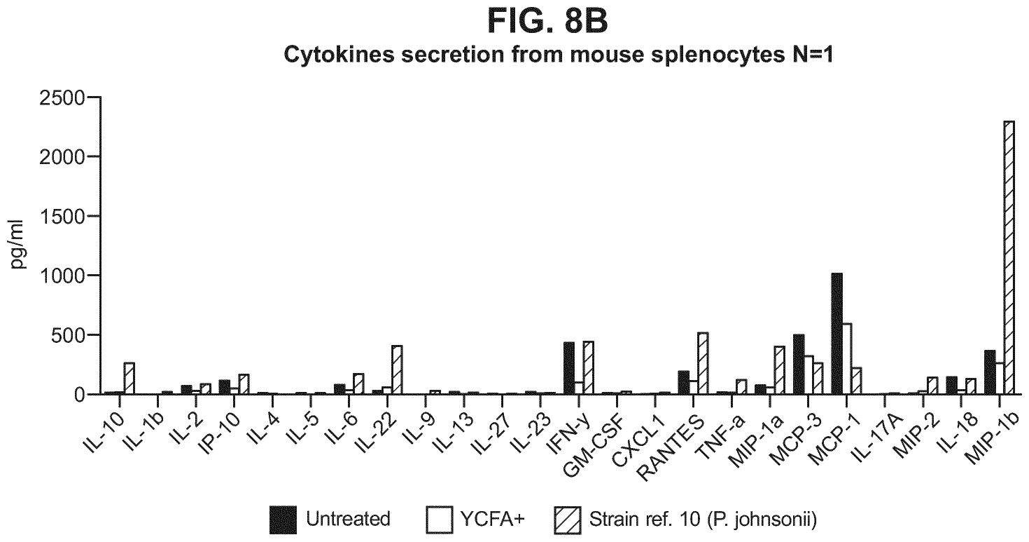

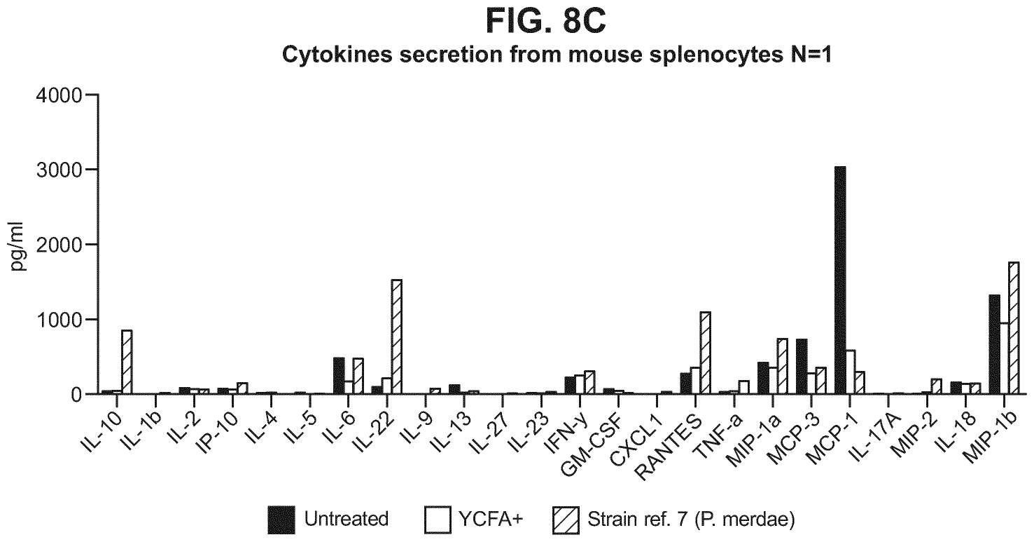

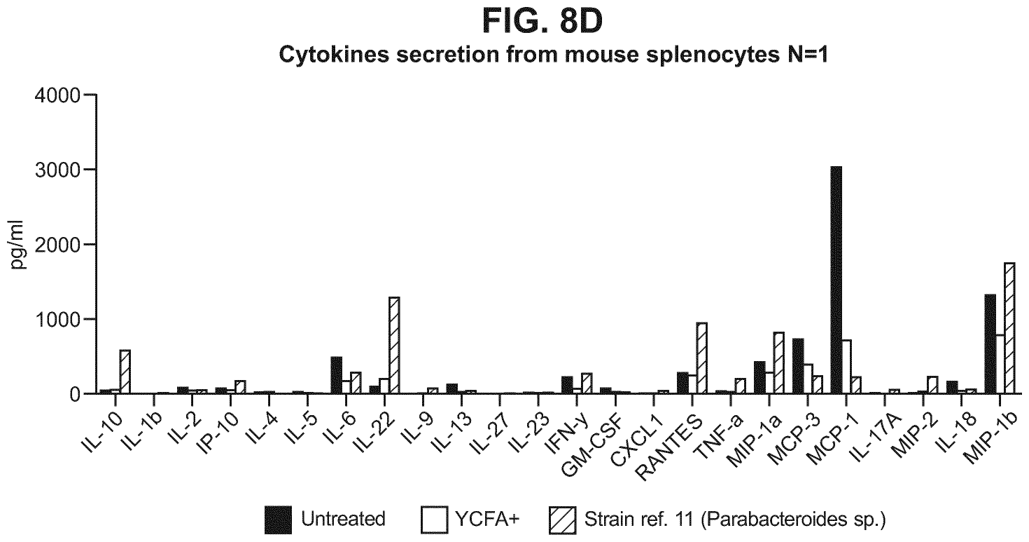

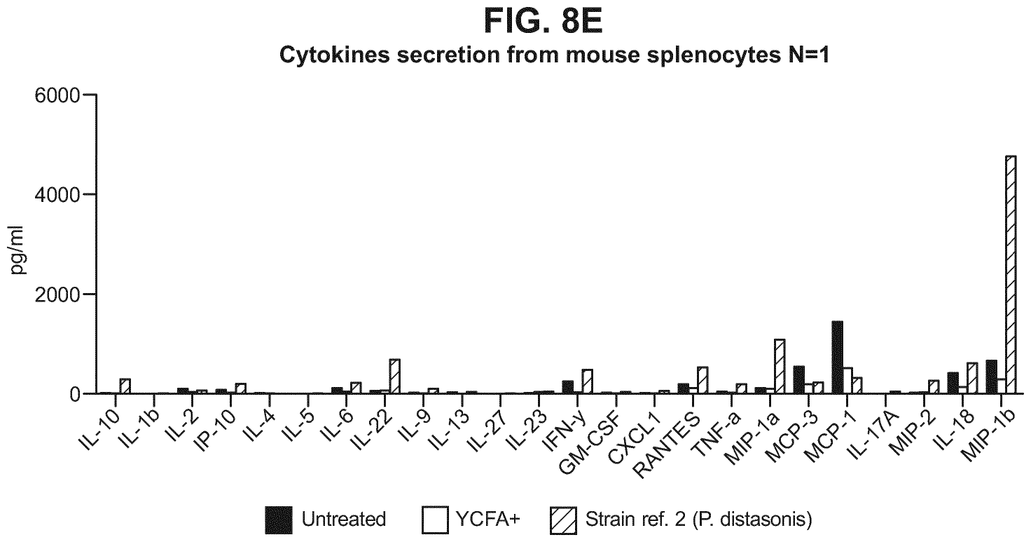

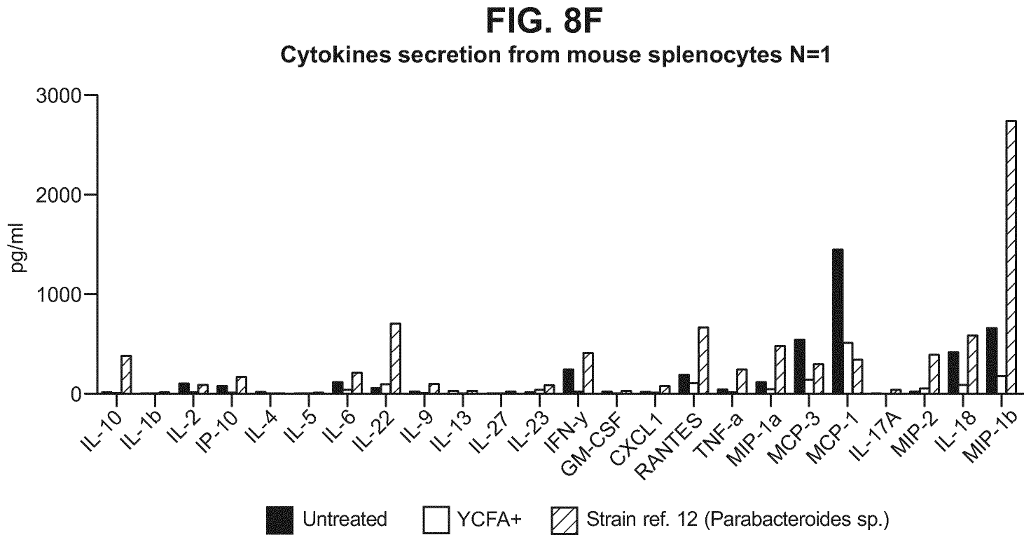

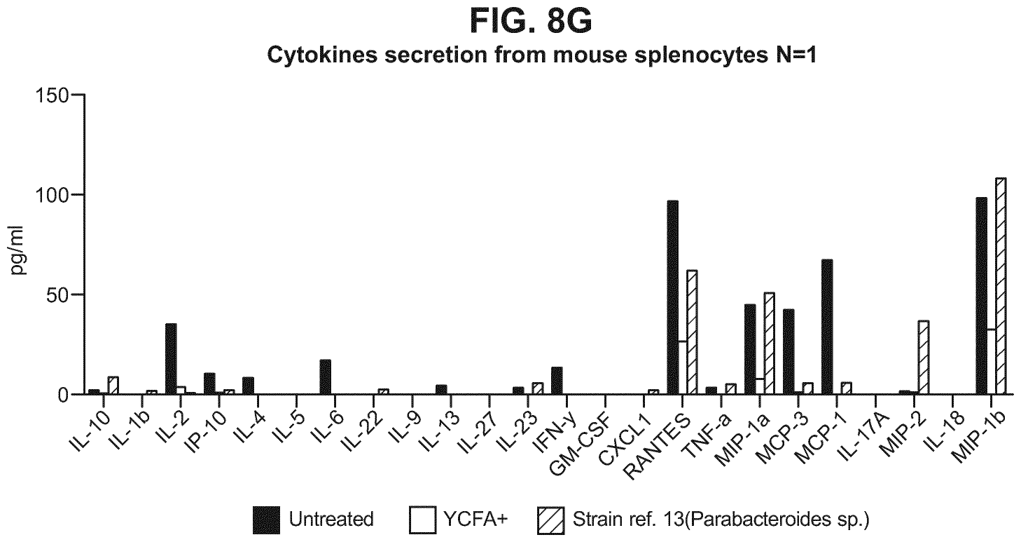

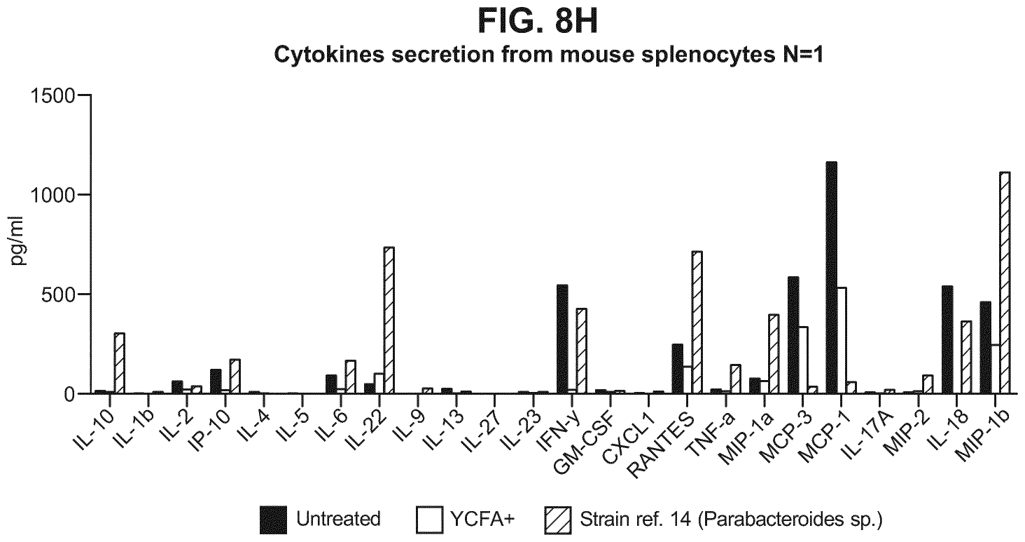

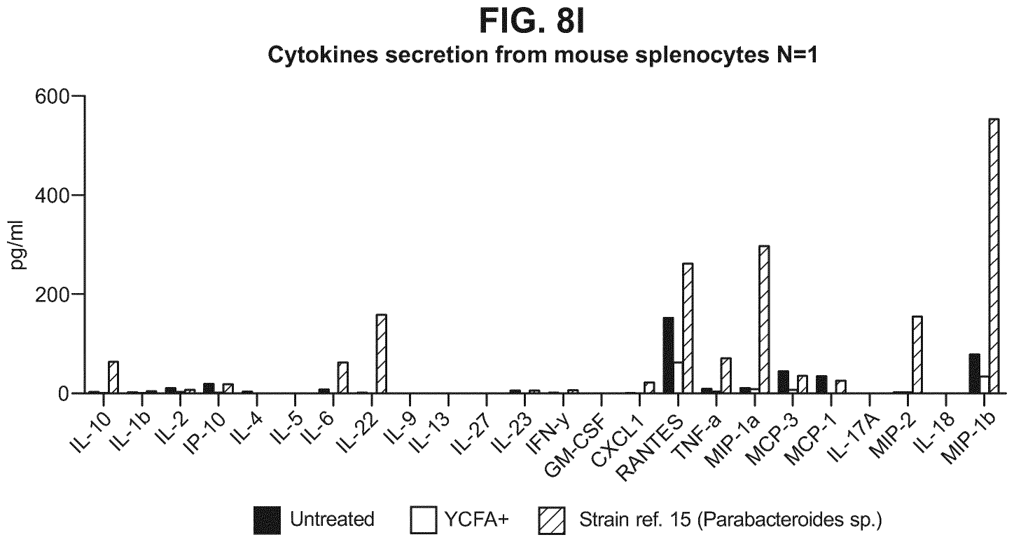

The Parabacteroides strains tested were: strain ref 2 (P. distasonis), strain ref 7 (P. merdae), strain ref 9 (P. distasonis), strain ref 10 (P. johnsonii), strain ref 11 (Parabacteroides sp.), strain ref 12 (Parabacteroides sp.), strain ref 13 (Parabacteroides sp.), strain ref 14 (Parabacteroides sp.) and strain ref 15 (Parabacteroides sp.). The results are shown in FIG. 8. Treatment of mouse splenocytes with supernatants from most Parabacteroides strains tested elicited greater secretion of the cytokines/chemokines IP-10, RANTES, TNF-α, MIP-1α, MIP-1B and MIP2, than the YCFA+media and untreated controls. Overall, these results demonstrate that treatment with Parabacteroides strains elicits immunostimulatory effects, indicating that they may act as vaccine adjuvants, therapeutics for treating, preventing or delaying immunosenescence, or adjunct therapeutics for enhancing a cell therapy such as CAR-T. As noted above in Example 6, many of the upregulated cytokines/chemokines (IP-10, RANTES, MIP-1α, MIP-1B and MIP2) can recruit immune cells, and thus act to create a localised immune response.

### Example 8—Short/Medium Chain Fatty Acid Production Profile of Parabacteroides distasonis Strain DM/120701

### Method

A pure culture of P. distasonis strain DSM 20701 was grown anaerobically in YCFA+broth. Short chain fatty acids (SCFAs) and medium chain fatty acids (MCFAs) from bacterial supernatants were analysed and quantified by MS Omics APS, Denmark. Samples were acidified using hydrochloride acid, and deuterium labelled internal standards were added. All samples were analyzed in a randomized order. Analysis was performed using a high polarity column (Zebron™ ZB-FFAP, GC Cap. Column 30 m×0.25 mm×0.25ⱼun) installed in a gas chromatograph (7890B, Agilent) coupled with a quadropole detector (5977B, Agilent). The system was controlled by ChemStation (Agilent). Raw data was converted to netCDF format using Chemstation (Agilent), before the data was imported and processed in Matlab R2014b (Mathworks, Inc.) using the PARADISe software.

### Results

P. distasonis strain DSM 20701 gave the following profile of short/medium chain fatty acids:

Short/medium chain fatty acid concentration (mM)

| | | | 2-methyl- | | 3-methyl- | | 4-methyl- | | |
|---|---|---|---|---|---|---|---|---|---|
| Acetic | Formic | Propanoic | propanoic | Butanoic | butanoic | Pentanoic | pentanoic | Hexanoic | Heptanoic |
| acid | acid | acid | acid | acid | acid | acid | acid | acid | acid |
| 0.9 | 0.5 | 5.2 | 0.2 | 0.0 | 0.3 | −0.1 | 0.0 | −0.1 | 0.0 |

### Example 9—Short/Medium Chain Fatty Acid Production Profile of Additional Parabacteroides Strains

### Method

Short/medium chain fatty acid production profiles for the strains detailed below were measured as per Example 8.

### Results

| | | Short/medium chain fatty acid concentration (mM) | | | | | | |
|---|---|---|---|---|---|---|---|---|
| Strain | | Succinic | Formic | Acetic | Propionic | Butyric | Valeric | Hexanoic |
| ref. | Species | acid | acid | acid | acid | acid | acid | acid |
| 2 | Parabacteroides sp. | Not | Not | 26.42 | 4.17 | Not | Not | Not |
| | | detected | detected | | | detected | detected | detected |
| 7 | P. merdae | 8.55 | Not | 20.34 | 8.73 | Not | Not | Not |
| | | | detected | | | detected | detected | detected |
| 9 | P. distasonis | Not | Not | 44.10 | 1.41 | Not | Not | Not |
| | | detected | detected | | | detected | detected | detected |
| 10 | P. johnsonii | Not | Not | 45.45 | 5.98 | Not | Not | Not |
| | | detected | detected | | | detected | detected | detected |
| 11 | Parabacteroides sp. | 6.74 | Not | 50.04 | 12.76 | Not | Not | Not |
| | | | detected | | | detected | detected | detected |
| 12 | Parabacteroides sp. | 14.70 | Not | 32.77 | 5.78 | Not | Not | Not |
| | | | detected | | | detected | detected | detected |
| 13 | Parabacteroides sp. | Not | Not | 43.11 | 17.70 | Not | Not | Not |
| | | detected | detected | | | detected | detected | detected |
| 14 | Parabacteroides sp. | 14.43 | Not | 10.99 | 5.96 | Not | Not | Not |
| | | | detected | | | detected | detected | detected |
| 15 | Parabacteroides sp. | 16.63 | 0.98 | 4.36 | 5.36 | Not | Not | Not |
| | | | | | | detected | detected | detected |

As can be seen, the different Parabacteroides strains tested consistently produced both acetic acid and propionic acid.

Sequences
SEQ ID NO: 1
(Parabacteroides distasonis gene for
16S ribosomal RNA, partial sequence, strain:
JCM 5825-AB3238922)

| | |
|---|---|
| 1 | agagtttgat cctggctcag gatgaacgct |
| | agcgacaggc ttaacacatg caagtcgagg |
| 61 | ggcagcgggg tgtagcaata caccgccggc |
| | gaccggcgca cgggtgagta acgcgtatgc |
| 121 | aacttgccta tcagaggggg ataacccggc |
| | gaaagtcgga ctaataccgc atgaagcagg |
| 181 | gatcccgcat gggaatattt gctaaagatt |
| | catcgctgat agataggcat gcgttccatt |
| 241 | aggcagttgg cggggtaacg gcccaccaaa |
| | ccgacgatgg ataggggttc tgagaggaag |
| 301 | gtcccccaca ttggtactga gacacggacc |
| | aaactcctac gggaggcagc agtgaggaat |
| 361 | attggtcaat gggcgtaagc ctgaaccagc |
| | caagtcgcgt gagggatgaa ggttctatgg |
| 421 | atcgtaaacc tcttttataa gggaataaag |
| | tgcgggacgt gtcccgtttt gtatgtacct |
| 481 | tatgaataag gatcggctaa ctccgtgcca |
| | gcagccgcgg taatacggag gatccgagcg |
| 541 | ttatccggat ttattgggtt taaagggtgc |
| | gtaggcggcc ttttaagtca gcggtgaaag |
| 601 | tctgtggctc aaccatagaa ttgccgttga |
| | aactgggggg cttgagtatg tttgaggcag |
| 661 | gcggaatgcg tggtgtagcg gtgaaatgca |
| | tagatatcac gcagaacccc gattgcgaag |
| 721 | gcagcctgcc aagccattac tgacgctgat |
| | gcacgaaagc gtggggatca aacaggatta |
| 781 | gataccctgg tagtccacgc agtaaacgat |
| | gatcactagc tgtttgcgat acactgtaag |
| 841 | cggcacagcg aaagcgttaa gtgatccacc |
| | tggggagtac gccggcaacg gtgaaactca |
| 901 | aaggaattga cgggggcccg cacaagcgga |
| | ggaacatgtg gtttaattcg atgatacgcg |
| 961 | aggaacctta cccgggtttg aacgcattcg |
| | gaccgaggtg gaaacacctt ttctagcaat |
| 1021 | agccgtttgc gaggtgctgc atggttgtcg |
| | tcagctcgtg ccgtgaggtg tcggcttaag |
| 1081 | tgccataacg agcgcaaccc ttgccactag |
| | ttactaacag gttaggctga ggactctggt |
| 1141 | gggactgcca gcgtaagctg cgaggaaggc |
| | ggggatgacg tcaaatcagc acggccctta |
| 1201 | catccggggc gacacacgtg ttacaatggc |
| | gtggacaaag ggaggccacc tggcgacagg |
| 1261 | gagcgaatcc ccaaaccacg tctcagttcg |
| | gatcggagtc tgcaacccga ctccgtgaag |
| 1321 | ctggattcgc tagtaatcgc gcatcagcca |
| | tggcgcggtg aatacgttcc cgggccttgt |
| 1381 | acacaccgcc cgtcaagcca tgggagccgg |
| | gggtacctga agtccgtaac cgaaaggatc |
| 1441 | ggcctagggt aaaactggtg actggggcta |
| | agtcgtaaca aggtaacc |

SEQ ID NO: 2
(Parabacteroides distasonis gene for 16S
ribosomal RNA, partial sequence,
strain: JCM 13400-AB238923)

| | |
|---|---|
| 1 | agagtttgat cctggctcag gatgaacgct |
| | agcgacaggc ttaacacatg caagtcgagg |
| 61 | ggcagcacag gtagcaatac cgggtggcga |
| | ccggcgcacg ggtgagtaac gcgtatgcaa |
| 121 | cttacctatc agagggggat aacccggcga |
| | aagtcggact aataccgcat gaagcagggg |
| 181 | ccccgcatgg ggatatttgc taaagattca |
| | tcgctgatag ataggcatgc gttccattag |
| 241 | gcagttggcg gggtaacggc ccaccaaacc |
| | gacgatggat aggggttctg agaggaaggt |
| 301 | cccccacatt ggtactgaga cacggaccaa |
| | actcctacgg gaggcagcag tgaggaatat |
| 361 | tggtcaatgg gcgtaagcct gaaccagcca |
| | agtcgcgtga gggatgaagg ttctatggat |
| 421 | cgtaaacctc ttttataagg gaataaagtg |
| | cgggacgtgt cctgttttgt atgtacctta |
| 481 | tgaataagga tcggctaact ccgtgccagc |
| | agccgcggta atacggagga tccgagcgtt |
| 541 | atccggattt attgggttta aagggtgcgt |
| | aggcggcctt ttaagtcagc ggtgaaagtc |
| 601 | tgtggctcaa ccatagaatt gccgttgaaa |
| | ctggggggct tgagtatgtt tgaggcaggc |
| 661 | ggaatgcgtg gtgtagcggt gaaatgctta |
| | gatatcacgc agaaccccga ttgcgaaggc |
| 721 | agcctgccaa gccatgactg acgctgatgc |
| | acgaaagcgt ggggatcaaa caggattaga |
| 781 | taccctggta gtccacgcag taaacgatga |
| | tcactagctg tttgcgatac agtgtaagcg |
| 841 | gcacagcgaa agcgttaagt gatccacctg |
| | gggagtacgc cggcaacggt gaaactcaaa |
| 901 | ggaattgacg ggggcccgca caagcggagg |
| | aacatgtggt ttaattcgat gatacgcgag |
| 961 | gaaccttacc cgggtttgaa cgcattcgga |
| | ccgaggtgga aacacctttt ctagcaatag |
| 1021 | ccgtttgcga ggtgctgcat ggttgtcgtc |
| | agctcgtgcc gtgaggtgtc ggcttaagtg |
| 1081 | ccataacgag cgcaaccctt gccactagtt |
| | actaacaggt aaagctgagg actctggtgg |
| 1141 | gactgccagc gtaagctgcg aggaaggcgg |
| | ggatgacgtc aaatcagcac ggcccttaca |
| 1201 | tccggggcga cacacgtgtt acaatggcgt |
| | ggacaaaggg aagccacctg gcgacaggga |
| 1261 | gcgaatcccc aaaccacgtc tcagttcgga |
| | tcggagtctg caacccgact ccgtgaagct |
| 1321 | ggattcgcta gtaatcgcgc atcagccatg |
| | gcgcggtgaa tacgttcccg ggccttgtac |
| 1381 | acaccgcccg tcaagccatg ggagccgggg |
| | gtacctgaag tccgtaaccg aaaggatcgg |
| 1441 | cctagggtaa aactggtgac tggggctaag |
| | tcgtaacaag gtaacc |

SEQ ID NO: 3
(Parabacteroides distasonis gene for 16S
ribosomal RNA, partial sequence,
strain: JCM 13401-AB238924)

| | |
|---|---|
| 1 | agagtttgat cctggctcag gatgaacgct |
| | agcgacaggc ttaacacatg caagtcgagg |
| 61 | ggcagcacag gtagcaatac ccgccggcga |
| | ccggcgcacg ggtgagtaac gcgtatgcaa |
| 121 | cttgcctatc agagggggat aacccggcga |
| | aagtcggact aataccgcat gaagcagggg |
| 181 | ccccgcatgg ggatatttgc taaagattca |
| | tcgctgatag ataggcatgc gttccattag |
| 241 | gcagttggcg gggtaacggc ccaccaaacc |
| | gacgatggat aggggttctg agaggaaggt |
| 301 | cccccacatt ggtactgaga cacggaccaa |
| | actcctacgg gaggcagcag tgaggaatat |
| 361 | tggtcaatgg gcgtaagcct gaaccagcca |
| | agtcgcgtga gggatgaagg ttctatggat |
| 421 | cgtaaacctc ttttataagg gaataaagtg |
| | tgggacgtgt cctgttttgt atgtacctta |
| 481 | tgaataagga tcggctaact ccgtgccagc |
| | agccgcggta atacggagga tccgagcgtt |
| 541 | atccggattt attgggttta aagggtgcgt |
| | aggcggcctt ttaagtcagc ggtgaaagtc |
| 601 | tgtggctcaa ccatagaatt gccgttgaaa |
| | ctgggaggct tgagtatgtt tgaggcaggc |
| 661 | ggaatgcgtg gtgtagcggt gaaatgctta |
| | gatatcacgc agaaccccga ttgcgaaggc |
| 721 | agcctgccaa gccatgactg acgctgatgc |
| | acgaaagcgt ggggatcaaa caggattaga |
| 781 | taccctggta gtccacgcag taaacgatga |
| | tcactagctg tttgcgatac actgtaagcg |
| 841 | gcacagcgaa agcgttaagt gatccacctg |
| | gggagtacgc cggcaacggt gaaactcaaa |
| 901 | ggaattgacg ggggcccgca caagcggagg |
| | aacatgtggt ttaattcgat gatacgcgag |
| 961 | gaaccttacc cgggtttgaa cgcattcgga |
| | ccgaggtgga aacacctttt ctagcaatag |
| 1021 | ccgtttgcga ggtgctgcat ggttgtcgtc |
| | agctcgtgcc gtgaggtgtc ggcttaagtg |
| 1081 | ccataacgag cgcaaccctt gccactagtt |
| | actaacaggt gatgctgagg actctggtgg |
| 1141 | gactgccagc gtaagctgcg aggaaggcgg |
| | ggatgacgtc aaatcagcac ggcccttaca |
| 1201 | tccggggcga cacacgtgtt acaatggcgt |
| | ggacaaaggg atgccacctg gcgacaggga |
| 1261 | gcgaatcccc aaaccacgtc tcagttcgga |
| | tcggagtctg caacccgact ccgtgaagct |
| 1321 | ggattcgcta gtaatcgcgc atcagccatg |
| | gcgcggtgaa tacgttcccg ggccttgtac |
| 1381 | acaccgcccg tcaagccatg ggagccgggg |
| | gtacctgaag tccgtaaccg aaaggatcgg |
| 1441 | cctagggtaa aactggtgac tggggctaag |
| | tcgtaacaag gtaacc |

SEQ ID NO: 4
(Parabacteroides distasonis gene for 16S
ribosomal RNA, partial sequence,
strain: JCM 13402-AB238925)

| | |
|---|---|
| 1 | agagtttgat cctggctcag gatgaacgct |
| | agcgacaggc ttaacacatg caagtcgagg |
| 61 | ggcagcacag gtagcaatac cgggtggcga |
| | ccggcgcacg ggtgagtaac gcgtatgcaa |
| 121 | cttacctatc agagggggat aacccggcga |
| | aagtcggact aataccgcat gaagcagggg |
| 181 | ccccgcatgg ggatatttgc taaagattca |
| | tcgctgatag ataggcatgc gttccattag |
| 241 | gcagttggcg gggtaacggc ccaccaaacc |
| | gacgatggat aggggttctg agaggaaggt |
| 301 | cccccacatt ggtactgaga cacggaccaa |
| | actcctacgg gaggcagcag tgaggaatat |
| 361 | tggtcaatgg gcgtaagcct gaaccagcca |
| | agtcgcgtga gggatgaagg ttctatggat |
| 421 | cgtaaacctc ttttataagg gaataaagtg |
| | cgggacgtgt cccgttttgt atgtacctta |
| 481 | tgaataagga tcggctaact ccgtgccagc |
| | agccgcggta atacggagga tccgagcgtt |
| 541 | atccggattt attgggttta aagggtgcgt |
| | aggcggcctt ttaagtcagc ggtgaaagtc |
| 601 | tgtggctcaa ccatagaatt gccgttgaaa |
| | ctgggaggct tgagtatgtt tgaggcaggc |
| 661 | ggaatgcgtg gtgtagcggt gaaatgctta |
| | gatatcacgc agaaccccga ttgcgaaggc |
| 721 | agcctgccaa gccatgactg acgctgatgc |
| | acgaaagcgt ggggatcaaa caggattaga |
| 781 | taccctggta gtccacgcag taaacgatga |
| | tcactagctg tttgcgatac actgtaagcg |
| 841 | gcacagcgaa agcgttaagt gatccacctg |
| | gggagtacgc cggcaacggt gaaactcaaa |
| 901 | ggaattgacg ggggcccgca caagcggagg |
| | aacatgtggt ttaattcgat gatacgcgag |
| 961 | gaaccttacc cgggtttgaa cgcattcgga |
| | ccgaggtgga aacacctttt ctagcaatag |
| 1021 | ccgtttgcga ggtgctgcat ggttgtcgtc |
| | agctcgtgcc gtgaggtgtc ggcttaagtg |
| 1081 | ccataacgag cgcaaccctt gccactagtt |
| | actaacaggt aaagctgagg actctggtgg |
| 1141 | gactgccagc gtaagctgcg aggaaggcgg |
| | ggatgacgtc aaatcagcac ggcccttaca |
| 1201 | tccggggcga cacacgtgtt acaatggcgt |
| | ggacaaaggg aggccacctg gcgacaggga |
| 1261 | gcgaatcccc aaaccacgtc tcagttcgga |
| | tcggagtctg caacccgact ccgtgaagct |
| 1321 | ggattcgcta gtaatcgcgc atcagccatg |
| | gcgcggtgaa tacgttcccg ggccttgtac |
| 1381 | acaccgcccg tcaagccatg ggagccgggg |
| | gtacctgaag tccgtaaccg aaaggatcgg |
| 1441 | cctagggtaa aactggtgac tggggctaag |
| | tcgtaacaag gtaacc |

SEQ ID NO: 5
(Parabacteroides distasonis gene for 16S
ribosomal RNA, partial sequence,
strain: JCM 13403-AB238926)

| | |
|---|---|
| 1 | agagtttgat cctggctcag gatgaacgct |
| | agcgacaggc ttaacacatg caagtcgagg |
| 61 | ggcagcacag gtagcaatac cgggtggcga |
| | ccggcgcacg ggtgagtaac gcgtatgcaa |
| 121 | cttacctatc agagggggat aacccggcga |
| | aagtcggact aataccgcat gaagcagggg |
| 181 | ccccgcatgg ggatatttgc taaagattca |
| | tcgctgatag ataggcatgc gttccattag |
| 241 | gcagttggcg gggtaacggc ccaccaaacc |
| | gacgatggat aggggttctg agaggaaggt |
| 301 | cccccacatt ggtactgaga cacggaccaa |
| | actcctacgg gaggcagcag tgaggaatat |
| 361 | tggtcaatgg gcgtaagcct gaaccagcca |
| | agtcgcgtga gggatgaagg ttctatggat |
| 421 | cgtaaacctc ttttataagg gaataaagtg |
| | tgggacgtgt cccgttttgt atgtacctta |
| 481 | tgaataagga tcggctaact ccgtgccagc |
| | agccgcggta atacggagga tccgagcgtt |
| 541 | atccggattt attgggttta aagggtgcgt |
| | aggcggcctt ttaagtcagc ggtgaaagtc |
| 601 | tgtggctcaa ccatagaatt gccgttgaaa |
| | ctgggaggct tgagtatgtt tgaggcaggc |
| 661 | ggaatgcgtg gtgtagcggt gaaatgctta |
| | gatatcacgc agaaccccga ttgcgaaggc |
| 721 | agcctgccaa gccatgactg acgctgatgc |
| | acgaaagcgt ggggatcaaa caggattaga |
| 781 | taccctggta gtccacgcag taaacgatga |
| | tcactagctg tttgcgatac attgtaagcg |
| 841 | gcacagcgaa agcgttaagt gatccacctg |
| | gggagtacgc cggcaacggt gaaactcaaa |
| 901 | ggaattgacg ggggcccgca caagcggagg |
| | aacatgtggt ttaattcgat gatacgcgag |
| 961 | gaaccttacc cgggtttgaa cgcattcgga |
| | ccgaggtgga aacacctttt ctagcaatag |
| 1021 | ccgtttgcga ggtgctgcat ggttgtcgtc |
| | agctcgtgcc gtgaggtgtc ggcttaagtg |
| 1081 | ccataacgag cgcaaccctt gccactagtt |
| | actaacaggt aaagctgagg actctggtgg |
| 1141 | gactgccagc gtaagctgcg aggaaggcgg |
| | ggatgacgtc aaatcagcac ggcccttaca |
| 1201 | tccggggcga cacacgtgtt acaatggcgt |
| | ggacaaaggg aggccacctg gcgacaggga |
| 1261 | gcgaatcccc aaaccacgtc tcagttcgga |
| | tcggagtctg caacccgact ccgtgaagct |
| 1321 | ggattcgcta gtaatcgcgc atcagccatg |
| | gcgcggtgaa tacgttcccg ggccttgtac |
| 1381 | acaccgcccg tcaagccatg ggagccgggg |
| | gtacctgaag tccgtaaccg aaaggatcgg |
| 1441 | cctagggtaa aactggtgac tggggctaag |
| | tcgtaacaag gtaacc |

SEQ ID NO: 6
(Parabacteroides distasonis gene for 16S
ribosomal RNA, partial sequence,
strain: JCM 13404-AB238927)

| | |
|---|---|
| 1 | agagtttgat cctggctcag gatgaacgct |
| | agcgacaggc ttaacacatg caagtcgagg |
| 61 | ggcagcacag gtagcaatac cgggtggcga |
| | ccggcgcacg ggtgagtaac gcgtatgcaa |
| 121 | cttacctatc agagggggat aacccggcga |
| | aagtcggact aataccgcat gaagcagggg |
| 181 | ccccgcatgg ggatatttgc taaagattca |
| | tcgctgatag ataggcatgc gttccattag |
| 241 | gcagttggcg gggtaacggc ccaccaaacc |
| | gacgatggat aggggttctg agaggaaggt |
| 301 | cccccacatt ggtactgaga cacggaccaa |
| | actcctacgg gaggcagcag tgaggaatat |
| 361 | tggtcaatgg gcgtaagcct gaaccagcca |
| | agtcgcgtga gggatgaagg ttctatggat |
| 421 | cgtaaacctc ttttataagg gaataaagtg |
| | tgggacgtgt cccgttttgt atgtacctta |
| 481 | tgaataagga tcggctaact ccgtgccagc |
| | agccgcggta atacggagga tccgagcgtt |
| 541 | atccggattt attgggttta aagggtgcgt |
| | aggcggcctt ttaagtcagc ggtgaaagtc |
| 601 | tgtggctcaa ccatagaatt gccgttgaaa |
| | ctgggaggct tgagtatgtt tgaggcaggc |
| 661 | ggaatgcgtg gtgtagcggt gaaatgctta |
| | gatatcacgc agaaccccga ttgcgaaggc |
| 721 | agcctgccaa gccatgactg acgctgatgc |
| | acgaaagcgt ggggatcaaa caggattaga |
| 781 | taccctggta gtccacgcag taaacgatga |
| | tcactagctg tttgcgatac attgtaagcg |
| 841 | gcacagcgaa agcgttaagt gatccacctg |
| | gggagtacgc cggcaacggt gaaactcaaa |
| 901 | ggaattgacg ggggcccgca caagcggagg |
| | aacatgtggt ttaattcgat gatacgcgag |
| 961 | gaaccttacc cgggtttgaa cgcattcgga |
| | ccgaggtgga aacacctttt ctagcaatag |
| 1021 | ccgtttgcga ggtgctgcat ggttgtcgtc |
| | agctcgtgcc gtgaggtgtc ggcttaagtg |
| 1081 | ccataacgag cgcaaccctt gccactagtt |
| | actaacaggt aaagctgagg actctggtgg |
| 1141 | gactgccagc gtaagctgcg aggaaggcgg |
| | ggatgacgtc aaatcagcac ggcccttaca |
| 1201 | tccggggcga cacacgtgtt acaatggcgt |
| | ggacaaaggg aggccacctg gcgacaggga |
| 1261 | gcgaatcccc aaaccacgtc tcagttcgga |
| | tcggagtctg caacccgact ccgtgaagct |
| 1321 | ggattcgcta gtaatcgcgc atcagccatg |
| | gcgcggtgaa tacgttcccg ggccttgtac |
| 1381 | acaccgcccg tcaagccatg ggagccgggg |
| | gtacctgaag tccgtaaccg aaaggatcgg |
| 1441 | cctagggtaa aactggtgac tggggctaag |
| | tcgtaacaag gtaacc |

SEQ ID NO: 7
(Parabacteroides merdae gene for 16S
ribosomal RNA, partial sequence,
strain: JCM 9497-AB238928)

| | |
|---|---|
| 1 | agagtttgat cctggctcag gatgaacgct |
| | agcgacaggc ttaacacatg caagtcgagg |
| 61 | ggcagcatga tttgtagcaa tacagattga |
| | tggcgaccgg cgcacgggtg agtaacgcgt |
| 121 | atgcaactta cctatcagag ggggatagcc |
| | cggcgaaagt cggattaata ccccataaaa |
| 181 | caggggtccc gcatgggaat atttgttaaa |
| | gattcatcgc tgatagatag gcatgcgttc |
| 241 | cattaggcag ttggcggggt aacggcccac |
| | caaaccgacg atggataggg gttctgagag |
| 301 | gaaggtcccc cacattggta ctgagacacg |
| | gaccaaactc ctacgggagg cagcagtgag |
| 361 | gaatattggt caatggccga gaggctgaac |
| | cagccaagtc gcgtgaagga agaaggatct |
| 421 | atggtttgta aacttctttt ataggggaat |
| | aaagtggagg acgtgtcctt ttttgtatgt |
| 481 | accctatgaa taagcatcgg ctaactccgt |
| | gccagcagcc gcggtaatac ggaggatgcg |
| 541 | agcgttatcc ggatttattg ggtttaaagg |
| | gtgcgtaggt ggtgatttaa gtcagcggtg |
| 601 | aaagtttgtg gctcaaccat aaaattgccg |
| | ttgaaactgg gttacttgag tgtgtttgag |
| 661 | gtaggcggaa tgcgtggtgt agcggtgaaa |
| | tgcatagata tcacgcagaa ctccgattgc |
| 721 | gaaggcagct tactaaacca taactgacac |
| | tgaagcacga aagcgtgggg atcaaacagg |
| 781 | attagatacc ctggtagtcc acgcagtaaa |
| | cgatgattac taggagtttg cgatacaatg |
| 841 | taagctctac agcgaaagcg ttaagtaatc |
| | cacctgggga gtacgccggc aacggtgaaa |
| 901 | ctcaaaggaa ttgacggggg cccgcacaag |
| | cggaggaaca tgtggtttaa ttcgatgata |
| 961 | cgcgaggaac cttacccggg tttgaacgta |
| | gtctgaccgg agtggaaaca ctccttctag |
| 1021 | caatagcaga ttacgaggtg ctgcatggtt |
| | gtcgtcagct cgtgccgtga ggtgtcggct |
| 1081 | taagtgccat aacgagcgca acccttatca |
| | ctagttacta acaggtgaag ctgaggactc |
| 1141 | tggtgagact gccagcgtaa gctgtgagga |
| | aggtggggat gacgtcaaat cagcacggcc |
| 1201 | cttacatccg gggcgacaca cgtgttacaa |
| | tggcatggac aaagggcagc tacctggcga |
| 1261 | caggatgcta atctccaaac catgtctcag |
| | ttcggatcgg agtctgcaac tcgactccgt |
| 1321 | gaagctggat tcgctagtaa tcgcgcatca |
| | gccatggcgc ggtgaatacg ttcccgggcc |
| 1381 | ttgtacacac cgcccgtcaa gccatgggag |
| | ccgggggtac ctgaagtccg taaccgcaag |
| 1441 | gatcggccta gggtaaaact ggtgactggg |
| | gctaagtcgt aacaaggtaa cc |

SEQ ID NO: 8
(Parabacteroides merdae gene for 16S
ribosomal RNA, partial sequence,
strain: JCM 13405-AB238929)

| | |
|---|---|
| 1 | agagtttgat cctggctcag gatgaacgct |
| | agcgacaggc ttaacacatg caagtcgagg |
| 61 | ggcagcatga tttgtagcaa tacagattga |
| | tggcgaccgg cgcacgggtg agtaacgcgt |
| 121 | atgcaactta cctatcagag ggggatagcc |
| | cggcgaaagt cggattaata ccccataaaa |
| 181 | caggggttcc gcatgggaat atttgttaaa |
| | gattcatcgc tgatagatag gcatgcgttc |
| 241 | cattaggcag ttggcggggt aacggcccac |
| | caaaccgacg atggataggg gttctgagag |
| 301 | gaaggtcccc cacattggta ctgagacacg |
| | gaccaaactc ctacgggagg cagcagtgag |
| 361 | gaatattggt caatggccga gaggctgaac |
| | cagccaagtc gcgtgaagga agaaggatct |
| 421 | atggtttgta aacttctttt ataggggaat |
| | aaagtggagg acgtgtcctt ttttgtatgt |
| 481 | accctatgaa taagcatcgg ctaactccgt |
| | gccagcagcc gcggtaatac ggaggatgcg |
| 541 | agcgttatcc ggatttattg ggtttaaagg |
| | gtgcgtaggt ggtgatttaa gtcagcggtg |
| 601 | aaagtttgtg gctcaaccat aaaattgccg |
| | ttgaaactgg gttacttgag tgtgtttgag |
| 661 | gtaggcggaa tgcgtggtgt agcggtgaaa |
| | tgcatagata tcacgcagaa ctccgattgc |
| 721 | gaaggcagct tactaaacca taactgacac |
| | tgaagcacga aagcgtgggg atcaaacagg |
| 781 | attagatacc ctggtagtcc acgcagtaaa |
| | cgatgattac taggagtttg cgatacaatg |
| 841 | taagctctac agcgaaagcg ttaagtaatc |
| | cacctgggga gtacgccggc aacggtgaaa |
| 901 | ctcaaaggaa ttgacggggg cccgcacaag |
| | cggaggaaca tgtggtttaa ttcgatgata |
| 961 | cgcgaggaac cttacccggg tttgaacgta |
| | gtctgaccgg agtggaaaca ctccttctag |
| 1021 | caatagcaga ttacgaggtg ctgcatggtt |
| | gtcgtcagct cgtgccgtga ggtgtcggct |
| 1081 | taagtgccat aacgagcgca acccttatca |
| | ctagttacta acaggtgaag ctgaggactc |
| 1141 | tggtgagact gccagcgtaa gctgtgagga |
| | aggtggggat gacgtcaaat cagcacggcc |
| 1201 | cttacatccg gggcgacaca cgtgttacaa |
| | tggcatggac aaagggcagc tacctggcga |
| 1261 | caggatgcta atctccaaac catgtctcag |
| | ttcggatcgg agtctgcaac tcgactccgt |
| 1321 | gaagctggat tcgctagtaa tcgcgcatca |
| | gccatggcgc ggtgaatacg ttcccgggcc |
| 1381 | ttgtacacac cgcccgtcaa gccatgggag |
| | ccgggggtac ctgaagtccg taaccgcaag |
| 1441 | gatcggccta gggtaaaact ggtgactggg |
| | gctaagtcgt aacaaggtaa cc |

SEQ ID NO: 9
(consensus 16S rRNA gene sequence for
Parabacteroides distasonis strain 755/NCIMB
42382)
AMCCGGGTGGCGACCGGCGCACGGGTGAGTAACGCGTATG
CAACTTGCCTATCAGAGGGGGATAACCCGGCGAAAGTCGG
ACTAATACCGCATGAAGCAGGGATCCCGCATGGGAATATT
TGCTAAAGATTCATCGCTGATAGATAGGCATGCGTTCCAT
TAGGCAGTTGGCGGGGTAACGGCCCACCAAACCGACGATG
GATAGGGGTTCTGAGAGGAAGGTCCCCCACATTGGTACTG
AGACACGGACCAAACTCCTACGGGAGGCAGCAGTGAGGAA
TATTGGTCAATGGGCGTGAGCCTGAACCAGCCAAGTCGCG
TGAGGGATGAAGGTTCTATGGATCGTAAACCTCTTTTATA
AGGGAATAAAGTGCGGGACGTGTCCCGTTTTGTATGTACC
TTATGAATAAGGATCGGCTAACTCCGTGCCAGCAGCCGCG
GTAATACGGAGGATCCGAGCGTTATCCGGATTTATTGGGT
TTAAAGGGTGCGTAGGCGGCCTTTTAAGTCAGCGGTGAAA
GTCTGTGGCTCAACCATAGAATTGCCGTTGAAACTGGGAG
GCTTGAGTATGTTTGAGGCAGGCGGAATGCGTGGTGTAGC
GGTGAAATGCATAGATATCACGCAGAACCCCGATTGCGAA
GGCAGCCTGCCAAGCCATTACTGACGCTGATGCACGAAAG
CGTGGGGATCAAACAGGATTAGATACCCTGGTAGTCCACG
CAGTAAACGATGATCACTAGCTGTTTGCGATACACTGTAA
GCGGCACAGCGAAAGCGTTAAGTGATCCACCTGGGGAGTA
CGCCGGCAACGGTGAAACTCAAAGGAATTGACGGGGGCCC
GCACAAGCGGAGGAACATGTGGTTTAATTCGATGATACGC
GAGGAACCTTACCCGGGTTTGAACGCATTCGGACMGAKGT
GGAAACACATTTTCTAGCAATAGCCATTTGCGAGGTGCTG
CATGGTTGTCGTCAGCTCGTGCCGTGAGGTGTCGGCTTAA
GTGCCATAACGAGCGCAACCCTTGCCACTAGTTACTAACA
GGTAAAGCTGAGGACTCTGGTGGGACTGCCAGCGTAAGCT
GCGAGGAAGGCGGGGATGACGTCAAATCAGCACGGCCCTT
ACATCCGGGGCGACACACGTGTTACAATGGCGTGGACAAA
GGGAAGCCACCTGGCGACAGGGAGCGAATCCCCAAACCAC
GTCTCAGTTCGGATCGGAGTCTGCAACCCGACTCCGTGAA
GCTGGATTCGCTAGTAATCGCGCATCAGCCATGGCGCGGT
GAATACGTTCCCGGGCCTTGTACACACCGCCCGTCAAGCC
ATGGGAGCCGGGGGTACCTGAAGTCCGTAACCGCGAGGAT
CGGCCTAGGGTAAAACTGGTGACTGGGGCTAAGTCGTACG
GGG
SEQ ID NO: 10
(Parabacteroides goldsteinii strain
DSMZ 19448/JCM13446, 16S ribosomal RNA
gene, partial sequence-GenBank: EU136697.1):

| | |
|---|---|
| 1 | gtttgatcct ggctcaggat gaacgctagc |
| | gacaggctta acacatgcaa gtcgaggggc |
| 61 | agcacgatgt agcaatacat tggtggcgac |
| | cggcgcacgg gtgagtaacg cgtatgcaac |
| 121 | ctacctatca gaggggaata acccggcgaa |
| | agtcggacta ataccgcata aaacaggggt |
| 181 | tccacatgga aatatttgtt aaagaattat |
| | cgctgataga tgggcatgcg ttccattaga |
| 241 | tagttggtga ggtaacggct caccaagtcc |
| | acgatggata ggggttctga gaggaaggtc |
| 301 | ccccacactg gtactgagac acggaccaga |
| | ctcctacggg aggcagcagt gaggaatatt |
| 361 | ggtcaatggg cgagagcctg aaccagccaa |
| | gtcgcgtgaa ggatgaagga tctatggttt |
| 421 | gtaaacttct tttatatggg aataaagtga |
| | ggaacgtgtt cctttttgta tgtaccatat |
| 481 | gaataagcat cggctaactc cgtgccagca |
| | gccgcggtaa tacggaggat gcgagcgtta |
| 541 | tccggattta ttgggtttaa agggtgcgta |
| | ggtggttaat taagtcagcg gtgaaagttt |
| 601 | gtggctcaac cataaaattg ccgttgaaac |
| | tggttgactt gagtatattt gaggtaggcg |
| 661 | gaatgcgtgg tgtagcggtg aaatgcatag |
| | atatcacgca gaactccgat tgcgaaggca |
| 721 | gcttactaaa ctataactga cactgaagca |
| | cgaaagcgtg gggatcaaac aggattagat |
| 781 | accctggtag tccacgcagt aaacgatgat |
| | tactagctgt ttgcgataca cagtaagcgg |
| 841 | cacagcgaaa gcgttaagta atccacctgg |
| | ggagacgccg gcaacggtga aactcaaagg |
| 901 | aattgacggg ggcccgcaca agcggaggaa |
| | catgtggttt aattcgatga tacgcgagga |
| 961 | accttacccg ggtttgaacg catattgaca |
| | gctctggaaa cagagtctct agtaatagca |
| 1021 | atttgcgagg tgctgcatgg ttgtcgtcag |
| | ctcgtgccgt gaggtgtcgg cttaagtgcc |
| 1081 | ataacgagcg caacccttat cactagttac |
| | taacaggtca tgctgaggac tctagtgaga |
| 1141 | ctgccagcgt aagctgtgag gaaggtgggg |
| | atgacgtcaa atcagcacgg cccttacatc |
| 1201 | cggggcgaca cacgtgttac aatggtgggg |
| | acaaagggca gctaccgtgt gagcggatgc |
| 1261 | gaatctccaa accccatctc agttcggatc |
| | gaagtctgca acccgacttc gtgaagctgg |
| 1321 | attcgctagt aatcgcgcat cagccatggc |
| | gcggtgaata cgttcccggg ccttgtacac |
| 1381 | accacccgtc aagccatggg agttgggggt |
| | acctaaagtc cgtaaccgca aggatcggcc |
| 1441 | tagggtaaaa ccgatgactg gggctaagtc |
| | gtaacaaggt agccgtaccg gaaggtgcgg |
| 1501 | ctggaacacc tcctttctgg agcgcagagt |
| | tcgttatcaa gttgactcag aggtattagt |
| 1561 | taacttgtac tacggttgaa tatgtataaa |
| | atatagatct accggcaata aagtgtcggc |
| 1621 | aagagagaaa aatgatgctg agggaaacca |
| | aggcaaagtt gacagtccta tagctcagtt |
| 1681 | ggttagagcg ctacactgat aatgtagagg |
| | tcggcagttc aactctgcct gggactacag |
| 1741 | aatctctaag agagaatttt gggggattag |
| | ctcagctggc tagagcatct gccttgcacg |
| 1801 | cagagggtca acggttcgaa tccgttattc |
| | tccacaaaaa gttaccgaga catcagaaac |
| 1861 | gtaaagtaac gacaagatct ttgacatgat |
| | ggacaacgta aaataaagta acaagagcaa |
| 1921 | gctgaagata tatcaatccg atttacccct |
| | gtggtaaccg gaaataagaa agtaagcaag |
| 1981 | ggcagacggt ggatgccttg gc |

SEQ ID NO: 11
(Parabacteroides goldsteinii strain
DSMZ29187/BS-C3-2 16S, ribosomal RNA
gene, partial sequence-Genbank GQ456205.2):

| | |
|---|---|
| 1 | ctggctcagg atgaacgcta gcgacaggct |
| | taacacatgc aagtcgaggg gcagcacgat |
| 61 | gtagcaatac attggtggcg accggcgcac |
| | gggtgagtaa cgcgtatgca acctacctat |
| 121 | cagaggggaa taacccggcg aaagtcggac |
| | taataccgca taaaacaggg gttccacatg |
| 181 | gaaatatttg ttaaagaatt atcgctgata |
| | gatgggcatg cgttccatta gatagttggt |
| 241 | gaggtaacgg ctcaccaagt ccacgatgga |
| | taggggttct gagaggaagg tcccccacac |
| 301 | tggtactgag acacggacca gactcctacg |
| | ggaggcagca gtgaggaata ttggtcaatg |
| 361 | ggcgagagcc tgaaccagcc aagtcgcgtg |
| | aaggatgaag gatctatggt ttgtaaactt |
| 421 | cttttatatg ggaataaagt gaggaaacgt |
| | gttccttttt gtatgtacca tatgaataag |
| 481 | catcggctaa cttccgtgcc agcagccgcg |
| | gtaatacgga ggatgcgagc gttatccgga |
| 541 | tttattgggt ttaaagggtg cgtaggtggt |
| | taattaagtc agcggtgaaa gtttgtggct |
| 601 | caaccataaa attgccgttg aaactggttg |
| | acttgagtat atttgaggta ggcggaatgc |
| 661 | gtggtgtagc ggtgaaatgc atagatatca |
| | cgcagaactc cgattgcgaa ggcagcttac |
| 721 | taaactataa ctgacactga agcacgaaag |
| | cgtggggatc aaacaggatt agataccctg |
| 781 | gtagtccacg cagtaaacga tgattactag |
| | ctgtttgcga tacacagtaa gcggcacagc |
| 841 | gaaagcgtta agtaatccac ctggggagta |
| | cgccggcaac ggtgaaactc aaaggaattg |
| 901 | acgggggccc gcacaagcgg aggaacatgt |
| | ggtttaattc gatgatacgc gaggaacctt |
| 961 | acccgggttt gaacgcattc ggaccggagt |
| | ggaaacactt cttctagtaa tagccgtttg |
| 1021 | cgaggtgctg catggttgtc gtcagctcgt |
| | gccgtgaggt gtcggcttaa gtgccataac |
| 1081 | gagcgcaacc cttatcacta gttactaaca |
| | ggtcaagctg aggactctag tgagactgcc |
| 1141 | agcgtaagct gtgaggaagg tggggatgac |
| | gtcaaatcag cacggccctt acatccgggg |
| 1201 | cgacacacgt gttacaatgg tggggacaaa |
| | gggcagctac ctggcgacag gatgctaatc |
| 1261 | tccaaacctc atctcagttc ggatcgaagt |
| | ctgcaacccg acttcgtgaa gctggattcg |
| 1321 | ctagtaatcg cgcatcagcc atggcgcggt |
| | gaatacgttc ccgggccttg tacacaccgc |
| 1381 | ccgtcaagcc atgggagttg ggggtaccta |
| | aagtccgtaa ccgcaagg |

SEQ ID NO: 12:
Strain ref. 1 (P. distasonis) 16S
ribosomal RNA gene, assembled
using Geneious:
AAGGCCGATCCTTGTCGGTTACGGACTTCAGGTACCCCCG
GCTCCCATGGCTTGACGGGCGGTGTGTACAAGGCCCGGGA
ACGTATTCACCGCGCCATGGCTGATGCGCGATTACTAGCG
AATCCAGCTTCACGGAGTCGGGTTGCAGACTCCGATCCGA
ACTGAGACGTGGTTTGGGGATTCGCTCCCTGTCACCAGGT
GGCCTCCCTTTGTCCACGCCATTGTAACACGTGTGTCGCC
CCGGATGTAAGGGCCGTGCTGATTTGACGTCATCCCCGCC
TTCCTCGCAGCTTACGCTGGCAGTCCCACCAGAGTCCTCA
GCTTTACCTGTTAGTAACTAGTGGCAAGGGTTGCGCTCGT
TATGGCACTTAAGCCGACACCTCACGGCACGAGCTGACGA
CAACCATGCAGCACCTCGCAAACGGCTATTGCTAGAAAAG
GTGTTTCCACCTCGGTCCGAATGCGTTCAAACCCGGGTAA
GGTTCCTCGCGTATCATCGAATTAAACCACATGTTCCTCC
GCTTGTGCGGGCCCCCGTCAATTCCTTTGAGTTTCACCGT
TGCCGGCGTACTCCCCAGGTGGATCACTTAACGCTTTCGC
TGTGCCGCTTACAGTGTATCGCAAACAGCTAGTGATCATC
GTTTACTGCGTGGACTACCAGGGTATCTAATCCTGTTTGA
TCCCCACGCTTTCGTGCATCAGCGTCAGTCATGGCTTGGC
AGGCTGCCTTCGCAATCGGGGTTCTGCGTGATATCTATGC
ATTTCACCGCTACACCACGCATTCCGCCTGCCTCAAACAT
ACTCAAGCCCCCCAGTTTCAACGGCAATTCTATGGTTGAG
CCACAGACTTTCACCGCTGACTTAAAAGGCCGCCTACGCA
CCCTTTAAACCCAATAAATCCGGATAACGCTCGGATCCTC
CGTATTACCGCGGCTGCTGGCACGGAGTTAGCCGATCCTT
ATTCATAAGGTACATACAAAACGGGACACGTCCCGCACTT
TATTCCCTTATAAAAGAGGTTTACGATCCATAGAACCTTC
ATCCCTCACGCGACTTGGCTGGTTCAGCCTCTCGGCCATT
GACCAATATTCCTCACTGCTGCCTCCCGTAGGAGTTTGGT
CCGTGTCTCAGTACCAATGTGGGGGACCTTCCTCTCAGAA
CCCCTATCCATCGTCGGTTTGGTGGGCCGTTACCCCGCCA
ACTGCCTAATGGAACGCATGCCTATCTATCAGCGATGAAT
CTTTAGCAAATATCCCCATGCGGGGCCCCTGCTTCATGCG
GTATTAGTCCGACTTTCGCCGGGTTATCCCCCTCTGATAG
GTAAGTTGCATACGCGTTACTCACCCGTGCGCCGGTCGCC
ACCCGGTATTGCTACCTGTGCTGCCGCCTCGACTGCA
SEQ ID NO: 13:
Strain ref. 2 (P. distasonis) 16S
ribosomal RNA gene, assembled using
Geneious:
AGGCCGATCCTTGTCGGTTACGGACTTCAGGTACCCCCGG
CTCCCATGGCTTGACGGGCGGTGTGTACAAGGCCCGGGAA
CGTATTCACCGCGCCATGGCTGATGCGCGATTACTAGCGA
ATCCAGCTTCACGGAGTCGGGTTGCAGACTCCGATCCGAA
CTGAGACGTGGTTTGGGGATTCGCTCCCTGTCGCCAGGTG
GCATCCCTTTGTCCACGCCATTGTAACACGTGTGTCGCCC
CGGATGTAAGGGCCGTGCTGATTTGACGTCATCCCCGCCT
TCCTCGCAGCTTACGCTGGCAGTCCCACCAGAGTCCTCAG
CTTTACCTGTTAGTAACTAGTGGCAAGGGTTGCGCTCGTT
ATGGCACTTAAGCCGACACCTCACGGCACGAGCTGACGAC
AACCATGCAGCACCTCGCAAATCGCTATTGCTAGAGGCTG
TGTTTCCACAGCGGTCCAAATGCGTTCAAACCCGGGTAAG
GTTCCTCGCGTATCATCGAATTAAACCACATGTTCCTCCG
CTTGTGCGGGCCCCCGTCAATTCCTTTGAGTTTCACCGTT
GCCGGCGTACTCCCCAGGTGGATCACTTAACGCTTTCGCT
GTGCCGCTTACCGTGTATCGCAAACAGCTAGTGATCATCG
TTTACTGCGTGGACTACCAGGGTATCTAATCCTGTTTGAT
CCCCACGCTTTCGTGCATCAGCGTCAGTCATGGCTTGGCA
GGCTGCCTTCGCAATCGGGGTTSTGCGTGATATCTAAGCA
TTTCACCGCTACACCACGCATTCCGCCTGCCTCAAACATA
CTCAAGCCCCCCAGTTTCAACGGCAATTCTATGGTTGAGC
CACAGACTTTCACCGCTGACTTAAAAGGCCGCCTACGCAC
CCTTTAAACCCAATAAATCCGGATAACGCTCGGATCCTCC
GTATTACCGCGGCTGCTGGCACGGAGTTAGCCGATCCTTA
TTCATAAGGTACATACAAAACGGGACACGTCCCGCACTTT
ATTCCCTTATAAAAGAGGTTTACGATCCATAGAACCTTCA
TCCCTCACGCGACTTGGCTGGTTCAGGCTTACGCCCATTG
ACCAATATTCCTCACTGCTGCCTCCCGTAGGAGTTTGGTC
CGTGTCTCAGTACCAATGTGGGGGACCTTCCTCTCAGAAC
CCCTATCCATCGTCGGTTTGGTGGGCCGTTACCCCGCCAA
CTGCCTAATGGAACGCATGCCTATCTATCAGCGATGAATC
TTTAGCAAATATCCCCATGCGGGACCCCTGCTTCATGCGG
TATTAGTCCGACTTTCGCCGGGTTATCCCCCTCTGATAGG
TAAGTTGCATACGCGTTACTCACCCGTGCGCCGGTCGCCA
CCCGGTATTGCTACCTGTGCTGCCCCTCGACTTGCATGTG
TAA
SEQ ID NO: 14:
Strain ref. 3 (Parabacteroides sp.) 16S
ribosomal RNA gene, assembled using
Geneious:
GGGCCCAATTTAACTAGGCCGATCCTTGCGGTTACGGACT
TCAGGTACCCCCGGCTCCCATGGCTTGACGGGCGGTGTGT
ACAAGGCCCGGGAACGTATTCACCGCGCCATGGCTGATGC
GCGATTACTAGCGAATCCAGCTTCACGGAGTCGAGTTGCA
GACTCCGATCCGAACTGAGACATGGTTTGGAGATTTGCAT
CACATCGCTGTGTAGCTGCCCTTTGTCCATGCCATTGTAA
CACGTGTGTCGCCCCGGATGTAAGGGCCGTGCTGATTTGA
CGTCATCCCCACCTTCCTCACAGCTTACGCTGGCAGTCTC
ACCAGAGTCCTCAGCTTGACCTGTTAGTAACTAGTGATAA
GGGTTGCGCTCGTTATGGCACTTAAGCCGACACCTCACGG
CACGAGCTGACGACAACCATGCAGCACYTCGCAAACGGTC
ATTGCTGAAAGGAGCGTTTCCACTCCGGTCCGAATGCGTT
CAAACCCGGGTAAGGTTCCTCGCGTATCATCGAATTAAAC
CACATGTTCCTCCGCTTGTGCGGGCCCCCGTCAATTCCTT
TGAGTTTCACCGTTGCCGGCGTACTCCCCAGGTGGATTAC
TTAACGCTTTCGCTGTAGAGCTTACTGTCTATCGCAAACT
CCTAGTAATCATCGTTTACTGCGTGGACTACCAGGGTATC
TAATCCTGTTTGATCCCCACGCTTTCGTGCTTCAGTGTCA
GTTATAGTTTAGTAAGCTGCCTTCGCAATCGGAGTTCTGC
GTGATATCTATGCATTTCACCGCTACACCACGCATTCCGC
CTACCTCAAATATACTCAAGTCATCCAGTTTCAACGGCAA
TTTTATGGTTGAGCCACAAACTTTCACCGCTGACTTAAAC
AACCACCTACGCACCCTTTAAACCCAATAAATCCGGATAA
CGCTCGCATCCTCCGTATTACCGCGGCTGCTGGCACGGAG
TTAGCCGATGCTTATTCATACGGTACATACAAAATGGGAC
ACGTCCCACACTTTATTCCCSKATAAAAGAAGTTTACAAA
CCATAGATCCTTCATCCTTCACGCGACTTGGCTGGTTCAG
CCTCCCGGCCATTGACCAATATTCCTCACTGCTGCCTCCC
GTAGGARTTTGGACCGTGTCTCAGTTCCAATGTGGGGGAC
CTTCCTCTCAGAACCCCTATCCATCGTCGGTTTGGTGGGC
CGTTACCCCGCCAACTGCCTAATGGAACGCATGCCTATCT
ATCAGCGATGAATCTTTAACAAATATTCCCATGCGGGACC
CCTGTTTTATGGAGCATTAATCCGACTTTCGCCGGGCTAT
TCCCCTCTGATAGGCAAGTTGCATACGCGTTACTCACCCG
TGCGCCGGTCGCCGGCAGGCATTGCTGCCCCCGCTGCCCC
TCGACTTGCATGGTTAGCCTCCAATTCCCC
SEQ ID NO: 15:
Strain ref. 4 (P. distasonis) 16S ribosomal
RNA gene, assembled using Geneious:
TAGGCCGATCCTTGCGGTTACGGACTTCAGGTACCCCCGG
CTCCCATGGCTTGACGGGCGGTGTGTACAAGGCCCGGGAA
CGTATTCACCGCGCCATGGCTGATGCGCGATTACTAGCGA
ATCCAGCTTCACGGAGTCGAGTTGCAGACTCCGATCCGAA
CTGAGACATGGTTTGGAGATTTGCATCACATCGCTGTGTA
GCTGCCCTTTGTCCATGCCATTGTAACACGTGTGTCGCCC
CGGATGTAAGGGCCGTGCTGATTTGACGTCATCCCCACCT
TCCTCACAGCTTACGCTGGCAGTCTCACCAGAGTCCTCAG
CTTGACCTGTTAGTAACTAGTGATAAGGGTTGCGCTCGTT
ATGGCACTTAAGCCGACACCTCACGGCACGAGCTGACGAC
AACCATGCAGCACCTCGCAAACGGTCATTGCTGAAAGGAG
CGTTTCCACTCCGGTCCGAATGCGTTCAAACCCGGGTAAG
GTTCCTCGCGTATCATCGAATTAAACCACATGTTCCTCCG
CTTGTGCGGGCCCCYGTCAATTCCTTTGAGTTTCACCGTT
GCCGGCGTACTCCCCAGGTGGATTACTTAACGCTTTCGCT
GTAGAGCTTACTGTCTATCGCAMACTCCTAGTAATCATCG
TTTACTGCGTGGACTACCAGGGTATCTAATCCTGTTTGAT
CCCCACGCTTTCGTGCTTCAGTGTCAGTTATAGTTTAGTA
AGCTGCCTTCGCAATCGGAGTTCTGCGTGATATCTATGCA
TTTCACCGCTACACCACGCATTCCGCCTACCTCAAATATA
CTCAAGTCATCCAGTTTCAACGGCAATTTTATGGTTGAGC
CACAAACTTTCACCGCTGACTTAAACAACCACCTACGCAC
CCTTTAAACCCAATAAATCCGGATAACGCTCGCATCCTCC
GTATTACCGCGGCTGCTGGCACGGAGTTAGCCGATGCTTA
TTCATACGGTACATACAAAATGGGACACGTCCCACACTTT
ATTCCCGTATAAAAGAAGTTTACAAACCATAGATCCTTCA
TCCTTCACGCGACTTGGCTGGTTCAGCCTCCCGGCCATTG
ACCAATATTCCTCACTGCTGCCTCCCGTAGGAGTTTGGAC
CGTGTCTCAGTTCCAATGTGGGGGACCTTCCTCTCAGAAC
CCCTATCCATCGTCGGTTTGGTGGGCCGTTACCCCGCCAA
CTGCCTAATGGAACGCATGCCTATCTATCAGCGATGAATC
TTTAACAAATATTCCCATGCGGGACCCCTGTTTTATGGAG
CATTAATCCGACTTTCGCCGGGCTATTCCCCTCTGATAGG
CAAGTTGCATACGCGTTACTCACCCGTGCGCCGGTCGCCG
GCAGGCATTGCTGCCCCCGCTGCCCCTCGACTTGCATGTG
TT
SEQ ID NO: 16:
Strain ref. 5 (P. distasonis) 16S ribosomal
RNA gene, assembled using Geneious:
GTAGGCCGATCCTCGCGGTTACGGACTTCAGGTACCCCCG
GCTCCCATGGCTTGACGGGCGGTGTGTACAAGGCCCGGGA
ACGTATTCACCGCGCCATGGCTGATGCGCGATTACTAGCG
AATCCAGCTTCACGGAGTCGGGTTGCAGACTCCGATCCGA
ACTGAGACGTGGTTTGGGGATTCGCTCCCTGTCGCCAGGT
GGCTTCCCTTTGTCCACGCCATTGTAACACGTGTGTCGCC
CCGGATGTAAGGGCCGTGCTGATTTGACGTCATCCCCGCC
TTCCTCGCAGCTTACGCTGGCAGTCCCACCAGAGTCCTCA
GCYTWACCTGTTAGTAACTAGTGGCAAGGGTTGCGCTCGT
TATGGCACTTAAGCCGACACCTCACGGCACGAGCTGACGA
CAACCATGCAGCACCTCGCAAACGGCTATTGCTAGAAAAG
GTGTTTCCACCTCGGTCCGAATGCGTTCAAACCCGGGTAA
GGTTCCTCGCGTATCATCGAATTAAACCACATGTTCCTCC
GCTTGTGCGGGCCCCCGTCAATTCCTTTGAGTTTCACCGT
TGCCGGCGTACTCCCCAGGTGGATCACTTAACGCTTTCGC
TGTGCCGCTTACACTGTATCGCAAACAGCTAGTGATCATC
GTTTACTGCGTGGACTACCAGGGTATCTAATCCTGTTTGA
TCCCCACGCTTTCGTGCATCAGCGTCAGTCATGGCTTGGC
AGGCTGCCTTCGCAATCGGGGTTCTGCGTGATATCTATGC
ATTTCACCGCTACACCACGCATTCCGCCTGCCTCAAACAT
ACTCAAGCCCCCCAGTTTCAACGGCAATTCTATGGTTGAG
CCACAGACTTTCACCGCTGACTTAAAAGGCCGCCTACGCA
CCCTTTAAACCCAATAAATCCGGATAACGCTCGGATCCTC
CGTATTACCGCGGCTGCTGGCACGGAGTTAGCCGATCCTT
ATTCATAAGGTACATACAAAACGGGACACGTCCTACACTT
TATTCCCTTATAAAAGAGGTTTACGATCCATAGAACCTTC
ATCCCTCACGCGACTTGGCTGGTTCAGGCTTACGCCCATT
GACCAATATTCCTCACTGCTGCCTCCCGTAGGAGTTTGGT
CCGTGTCTCAGTACCAATGTGGGGGACCTTCCTCTCAGAA
CCCCTATCCATCGTCGGTTTGGTGGGCCGTTACCCCGCCA
ACTGCCTAATGGAACGCATGCMTATMTATCAGCGATGWAT
CTTKMGCAAATATCCCCRTGCGGGGCCCGTGCTTCRTGCG
GTATTAGTCMGACTTTCGCCGGGTTATCCCCCTCTGATAG
GYAAGTTGCATACGCGTTACTCACCCGTGCGCCGGTCGCC
RGCCGCGGTATCTGCTACCCCGCGCTGCCCCTCGACTTGC
ATGGT
SEQ ID NO: 17:
Strain ref. 6 (P. distasonis) 16S ribosomal
RNA gene, assembled using Geneious:
GATCCTCGCGGTTACGGACTTCAGGTACCCCCGGCTCCCA
TGGCTTGACGGGCGGTGTGTACAAGGCCCGGGAACGTATT
CACCGCGCCATGGCTGATGCGCGATTACTAGCGAATCCAG
CTTCACGGAGTCGGGTTGCAGACTCCGATCCGAACTGAGA
CGTGGTTTGGGGATTCGCTCCCTGTCGCCAGGTGGCCTCC
CTTTGTCCACGCCATTGTAACACGTGTGTCGCCCCGGATG
TAAGGGCCGTGCTGATTTGACGTCATCCCCGCCTTCCTCG
CAGCTTACGCTGGCAGTCCCACCAGAGTCCTCAGCATCAC
CTGTTAGTAACTAGTGGCAAGGGTTGCGCTCGTTATGGCA
CTTAAGCCGACACCTCACGGCACGAGCTGACGACAACCAT
GCAGCACCTCGCAAACGGCTATTGCTAGAAAAGGTGTTTC
CACCTCGGTCCGAATGCGTTCAAACCCGGGTAAGGTTCCT
CGCGTATCATCGAATTAAACCACATGTTCCTCCGCTTGTG
CGGGCCCCCGTCAATTCCTTTGAGTTTCACCGTTGCCGGC
GTACTCCCCAGGTGGATCACTTAACGCTTTCGCTGTGCCG
CTTACAGTGTATCGCAAACAGCTAGTGATCATCGTTTACT
GCGTGGACTACCAGGGTATCTAATCCTGTTTGATCCCCAC
GCTTTCGTGCATCAGCGTCAGTCATGGCTTGGCAGGCTGC
CTTCGCAATCGGGGTTCTGCGTGATATCTATGCATTTCAC
CGCTACACCACGCATTCCGCCTGCCTCAAACATACTCAAG
CCCCCCAGTTTCAACGGCAATTCTATGGTTGAGCCACAGA
CTTTCACCGCTGACTTAAAAGGCCGCCTACGCACCCTTTA
AACCCAATAAATCCGGATAACGCTCGGATCCTCCGTATTA
CCGCGGCTGCTGGCACGGAGTTAGCCGATCCTTATTCATA
AGGTACATACAAAACGGGACACGTCCCGCACTTTATTCCC
TTATAAAAGAGGTTTACGATCCATAGAACCTTCATCCCTC
ACGCGACTTGGCTGGTTCAGCCTTTCGGCCATTGACCAAT
ATTCCTCACTGCTGCCTCCCGTAGGAGTTTGGTCCGTGTC
TCAGTACCAATGTGGGGGACCTTCCTCTCAGAACCCCTAT
CCATCGTCGGTTTGGTGGGCCGTTACCCCGCCAACTGCCT
AATGGAACGCATGCCTATCTATCAGCGATGAATCTTTAGC
AAATATCCCCATGCGGGGCCCCTGCTTCATGCGGTATTAG
TCCGACTTTCGCCGGGTTATCCCCCTCTGATAGGTAAGTT
GCATACGCGTTACTCACCCGTGCGCCGGTCGCCACCCGGT
ATTGC
SEQ ID NO: 18:
Strain ref. 7 (P. merdae)16S ribosomal
RNA gene, assembled using Geneious:
TTAAATAGGCCGATCCTTGCGGTTACGGACTTCAGGTACC
CCCGGCTCCCATGGCTTGACGGGCGGTGTGTACAAGGCCC
GGGAACGTATTCACCGCGCCATGGCTGATGCGCGATTACT
AGCGAATCCAGCTTCACGGAGTCGAGTTGCAGACTCCGAT
CCGAACTGAGACATGGTTTGGAGATTAGCATCCTGTCACC
AGGTAGCTGCCCTTTGTCCATGCCATTGTAACACGTGTGT
CGCCCCGGATGTAAGGGCCGTGCTGATTTGACGTCATCCC
CACCTTCCTCACAGCTTACGCTGGCAGTCTCACCAGAGTC
CTCAGCTTCACCTGTTAGTAACTAGTGATAAGGGTTGCGC
TCGTTATGGCACTTAAGCCGACACCTCACGGCACGAGCTG
ACGACAACCATGCAGCACCTCGTAATCTGCTATTGCTAGA
AAGAGTGTTTCCACTCCGGTCAGACTACGTTCAAACCCGG
GTAAGGTTCCTCGCGTATCATCGAATTAAACCACATGTTC
CTCCGCTTGTGCGGGCCCCCGTCAATTCCTTTGAGTTTCA
CCGTTGCCGGCGTACTCCCCAGGTGGATTACTTAACGCTT
TCGCTGTAGAGCTTACATTGTATCGCAAACTCCTAGTAAT
CATCGTTTACTGCGTGGACTACCAGGGTATCTAATCCTGT
TTGATCCCCACGCTTTCGTGCTTCAGTGTCAGTTATGGTT
TAGTAAGCTGCCTTCGCAATCGGAGTTCTGCGTGATATCT
ATGCATTTCACCGCTACACCACGCATTCCGCCTACCTCAA
ACACACTCAAGTAACCCAGTTTCAACGGCAATTTTATGGT
TGAGCCACAAACTTTCACCGCTGACTTAAATCACCACCTA
CGCACCCTTTAAACCCAATAAATCCGGATAACGCTCGCAT
CCTCCGTATTACCGCGGCTGCTGGCACGGAGTTAGCCGAT
GCTTATTCATAGGGTACATACAAAAAAGGACACGTCCTCC
ACTTTATTCCCCTATAAAAGAAGTTTACAAACCATAGATC
CTTCTTCCTTCACGCGACTTGGCTGGTTCAGCCTCTCGGC
CATTGACCAATATTCCTCACTGCTGCCTCCCGTAGGAGTT
TGGTCCGTGTCTCAGTACCAATGTGGGGGACCTTCCTCTC
AGAACCCCTATCCATCGTCGGTTTGGTGGGCCGTTACCCC
GCCAACTGCCTAATGGAACGCATGCCTATCTATCAGCGAT
GAATCTTTAACAAATATTCCCATGCGGGACCCCTGTTTTA
TGGGGTATTAATCCGACTTTCGCCGGGCTATCCCCCTCTG
ATAGGTAAGTTGCATACGCGTTACTCACCCGTGCGCCGGT
CGCCATCAATCTGTATTGCTACAAATCATGCTGCCCCTCG
ACTTGCATGGTTAAG
SEQ ID NO: 19
Strain ref. 9 (P. distasonis) 16S ribosomal
RNA gene, assembled using Geneious:
GATCTCGCGGTTTACGGACTTCAGGTACCCCCGGCTCCCA
TGGCTTGACGGGCGGTGTGTACAAGGCCCGGGAACGTATT
CACCGCGCCATGGCTGATGCGCGATTACTAGCGAATCCAG
CTTCACGGAGTCGGGTTGCAGACTCCGATCCGAACTGAGA
CGTGGTTTGGGGATTCGCTCCCTGTCGCCAGGTGGCCTCC
CTTTGTCCACGCCATTGTAACACGTGTGTCGCCCCGGATG
TAAGGGCCGTGCTGATTTGACGTCATCCCCGCCTTCCTCG
CAGCTTACGCTGGCAGTCCCACCAGAGTCCTCAGCATCAC
CTGTTAGTAACTAGTGGCAAGGGTTGCGCTCGTTATGGCA
CTTAAGCCGACACCTCACGGCACGAGCTGACGACAACCAT
GCAGCACCTCGCAAACGGCTATTGCTAGAAAAGGTGTTTC
CACCTCGGTCCGAATGCGTTCAAACCCGGGTAAGGTTCCT
CGCGTATCATCGAATTAAACCACATGTTCCTCCGCTTGTG
CGGGCCCCCGTCAATTCCTTTGAGTTTCACCGTTGCCGGC
GTACTCCCCAGGTGGATCACTTAACGCTTTCGCTGTGCCG
CTTACAGTGTATCGCAAACAGCTAGTGATCATCGTTTACT
GCGTGGACTACCAGGGTATCTAATCCTGTTTGATCCCCAC
GCTTTCGTGCATCAGCGTCAGTCATGGCTTGGCAGGCTGC
CTTCGCAATCGGGGTTCTGCGTGATATCTATGCATTTCAC
CGCTACACCACGCATTCCGCCTGCCTCAAACATACTCAAG
CCCCCCAGTTTCAACGGCAATTCTATGGTTGAGCCACAGA
CTTTCACCGCTGACTTAAAAGGCCGCCTACGCACCCTTTA
AACCCAATAAATCCGGATAACGCTCGGATCCTCCGTATTA
CCGCGGCTGCTGGCACGGAGTTAGCCGATCCTTATTCATA
AGGTACATACAAAACGGGACACGTCCCGCACTTTATTCCC
TTATAAAAGAGGTTTACGATCCATAGAACCTTCATCCCTC
ACGCGACTTGGCTGGTTCAGCCTTTCGGCCATTGACCAAT
ATTCCTCACTGCTGCCTCCCGTAGGAGTTTGGTCCGTGTC
TCAGTACCAATGTGGGGGACCTTCCTCTCAGAACCCCTAT
CCATCGTCGGTTTGGTGGGCCGTTACCCCGCCAACTGCCT
AATGGAACGCATGCCTATCTATCAGCGATGAATCTTTAGC
AAATATCCCCATGCGGGGCCCCTGCTTCATGCGGTATTAG
TCCGACTTTCGCCGGGTTATCCCCCTCTGATAGGTAAGTT
GCATACGCGTTACTCACCCGTGCGCCGGTCGCCACCCGGT
ATTGCTACCTGGTGCTGCCCCCTCGACTGC
SEQ ID NO: 20
Strain ref. 11 (Parabacteroides sp.) 16S
ribosomal RNA gene, assembled using
Geneious:
CCGATCCTTTCGGTTACGGACTTCAGGTACCCCCGGCTCC
CATGGCTTGACGGGCGGTGTGTACAAGGCCCGGGAACGTA
TTCACCGCGCCATGGCTGATGCGCGATTACTAGCGAATCC
AGCTTCACGGAGTCGGGTTGCAGACTCCGATCCGAACTGA
GACGTGGTTTGGGGATTCGCTCCCTGTCGCCAGGTGGCCT
CCCTTTGTCCACGCCATTGTAACACGTGTGTCGCCCCGGA
TGTAAGGGCCGTGCTGATTTGACGTCATCCCCGCCTTCCT
CGCAGCTTACGCTGGCAGTCCCACCAGAGTCCTCAGCATC
ACCTGTTAGTAACTAGTGGCAAGGGTTGCGCTCGTTATGG
CACTTAAGCCGACACCTCACGGCACGAGCTGACGACAACC
ATGCAGCACCTCGCAAATCGCTATCGCTAGAGACTCTGTT
TCCAGAGCTGTCGAAATGCGTTCAAACCCGGGTAAGGTTC
CTCGCGTATCATCGAATTAAACCACATGTTCCTCCGCTTG
TGCGGGCCCCCGTCAATTCCTTTGAGTTTCACCGTTGCCG
GCGTACTCCCCAGGTGGATCACTTAACGCTTTCGCTGTGC
CGCTTACAGTGTATCGCAAACAGCTAGTGATCATCGTTTA
CTGCGTGGACTACCAGGGTATCTAATCCTGTTTGATCCCC
ACGCTTTCGTGCATCAGCGTCAGTAATGGCTTGGCAGGCT
GCCTTCGCAATCGGGGTTCTGCGTGATATCTATGCATTTC
ACCGCTACACCACGCATTCYGCCTGCCTCAAACATACTCA
AGCCCCCCAGTTTCAACGGCAATTCTATGGTTGAGCCACA
GACTTTCACCGCTGACTTAAAAGGCCGCCTACGCACCCTT
TAAACCCAATAAATCCGGATAACGCTCGGATCCTCCGTAT
TACCGCGGCTGCTGGCACGGAGTTAGCCGATCCTTATTCA
TAAGGTACATACMAAACGGGACACGTCCCGCACTTTATTC
CCTTATAAAAGAGGTTTACGATCCATAGAACCTTCATCCC
TCACGCGACTTGGCTGGTTCAGSCTCTCGCCCATTGACCA
ATATTCCTCACTGCTGCCTCCCGTAGGAGTTTGGTCCGTG
TCTCAGTACCAATGTGGGGGACCTTCCTCTCAGAACCCCT
ATCCATCGTCGGTTTGGTGGGCCGTTACCCCGCCAACTGC
CTAATGGAACGCMWGCCKATYTATCAGCGAWGAATCTTTA
GCAAATATCCCCATGCGGGGCCCCTGCTTCMTGCGGTATT
AGTCCGACTTTCGCCGGGTTATCCCCCTCTGATAGGCAAG
TWGCATACGCGTTACTCACCCGTGCGCCGGTCGCCACCCG
GTATTGCTACCCTGYGCTGCCCCTCGACTTGCATGKTAA
SEQ ID NO: 21
Strain ref. 12 (Parabacteroides sp.) 16S
ribosomal RNA gene, assembled using
Geneious:
GCGCGGTTTAACTAGGCCGATCCTTTCGGTTACGGACTTC
AGGTACCCCCGGCTCCCATGGCTTGACGGGCGGTGTGTAC
AAGGCCCGGGAACGTATTCACCGCGCCATGGCTGATGCGC
GATTACTAGCGAATCCAGCTTCACGGAGTCGGGTTGCAGA
CTCCGATCCGAACTGAGACGTGGTTTGGGGATTCGCTCCC
TGTCGCCAGGTGGCCTCCCTTTGTCCACGCCATTGTAACA
CGTGTGTCGCCCCGGATGTAAGGGCCGTGCTGATTTGACG
TCATCCCCGCCTTCCTCGCAGCTTACGCTGGCAGTCCCAC
CAGAGTCCTCAGCATCACCTGTTAGTAACTAGTGGCAAGG
GTTGCGCTCGTTATGGCACTTAAGCCGACACCTCACGGCA
CGAGCTGACGACAACCATGCAGCACCTCGCAAATCGCTAT
CGCTAGAGACTCTGTTTCCAGAGCTGTCGAAATGCGTTCA
AACCCGGGTAAGGTTCCTCGCGTATCATCGAATTAAACCA
CATGTTCCTCCGCTTGTGNCGGGCCCCCGTCAATTCCTTT
GAGTTTCACCGTTGCCGGCGTAYTCCCCAGGTGGATCACT
TAACGCTTTCGCTGTGCCGCTTACAGTGTATCGCAAACAG
CTAGTGATCATCGTTTACTGCGTGGACTACCAGGGTATCT
AATCCTGTTTGATCCCCACGCTTTCGTGCATCAGCGTCAG
TAATGGCTTGGCAGGCTGCCTTCGCAATCGGGGTTCTGCG
TGATATCTATGCATTTCACCGCTACACCACGCATTCCGCC
TGCCTCAAACATACTCAAGCCCCCCANTTTCAACGGCAAT
TCTATGGTTGAGCCACAGACTTTCACCGCTGACTTAAAAG
GCCGCCTACGCACCCTTTAAACCCAATAAATCCGGATAAC
GCTCGGATCCTCCGTATTACCGCGGCTGCTGGCACGGAGT
TAGCCGATCCTTATTCATAAGGTACATACAAAACGGGACA
CGTCCCGCACTTTATTCCCTTATAAAAGAGGTTTACGATC
CATAGAACCTTCATCCCTCACGCGACTTGGCTGGTTCAGC
CTTTCGGCCATTGACCAATATTCCTCACTGCTGCCTCCCG
TAGGAGTTTGGTCCGTGTCTCAGTACCAATGTGGGGGACC
TTCCTCTCAGAACCCCTATCCATTGTCGGTTTGGTGGGCC
GTTACCCCGCCAACTGCCTAATGGAACGCATGCCTATCTA
TCAGCGATGAATCTTTAGCAAATATCCCCATGCGGGGCCC
CTGCTTCATGCGGTATTAGTCCGACTTTCGCCGGGTTATC
CCCCTCTGATAGGCAAGTTGCATACGCGTTACTCACCCGT
GCGCCGGTCGCCGAGCCGCGGTATTGCTACCCTCGTGCTG
CCCCTCGACTTGCATGGTTAGCCTCCATCCC
SEQ ID NO: 22
Strain ref. 14 (Parabacteroides sp.) 16S
ribosomal RNA gene, assembled using
Geneious:
CTTAGGCCGATCCCTCGCGGTTCGGACTTCAGGTACCCCC
GGCTCCCATGGCTTGACGGGCGGTGTGTACAAGGCCCGGG
AACGTATTCACCGCGCCATGGCTGATGCGCGATTACTAGC
GAATCCAGCTTCACGGAGTCGGGTTGCAGACTCCGATCCG
AACTGAGACGTGGTTTGGGGATTCGCTCCCTGTCGCCAGG
TGGCCTCCCTTTGTCCACGCCATTGTAACACGTGTGTCGC
CCCGGATGTAAGGGCCGTGCTGATTTGACGTCATCCCCGC
CTTCCTCGCAGCTTACGCTGGCAGTCCCACCAGAGTCCTC
AGCATCACCTGTTAGTAACTAGTGGCAAGGGTTGCGCTCG
TTATGGCACTTAAGCCGACACCTCACGGCACGAGCTGACG
ACAACCATGCAGCACCTCGCAAACGGCTATTGCTAGAAAA
GGTGTTTCCACCTCGGTCCGAATGCGTTCAAACCCGGGTA
AGGTTCCTCGCGTATCATCGAATTAAACCACATGTTCCTC
CGCTTGTGCGGGCCCCCGTCAATTCCTTTGAGTTTCACCG
TTGCCGGCGTACTCCCCAGGTGGATCACTTAACGCTTTCG
CTGTGCCGCTTACAGTGTATCGCAAACAGCTAGTGATCAT
CGTTTACTGCGTGGACTACCAGGGTATCTAATCCTGTTTG
ATCCCCACGCTTTCGTGCATCAGCGTCAGTCATGGCTTGG
CAGGCTGCCTTCGCAATCGGGGTTCTGCGTGATATCTATG
CATTTCACCGCTACACCACGCATTCCGCCTGCCTCAAACA
TACTCAAGCCCCCCAGTTTCAACGGCAATTCTATGGTTGA
GCCACAGACTTTCACCGCTGACTTAAAAGGCCGCCTACGC
ACCCTTTAAACCCAATAAATCCGGATAACGCTCGGATCCT
CCGTATTACCGCGGCTGCTGGCACGGAGTTAGCCGATCCT
TATTCATAAGGTACATACAAAACGGGACACGTCCCGCACT
TTATTCCCTTATAAAAGAGGTTTACGATCCATAGAACCTT
CATCCCTCACGCGACTTGGCTGGTTCAGCCTTTCGGCCAT
TGACCAATATTCCTCACTGCTGCCTCCCGTAGGAGTTTGG
TCCGTGTCTCAGTACCAATGTGGGGGACCTTCCTCTCAGA
ACCCCTATCCATCGTCGGTTTGGTGGGCCGTTACCCCGCC
AACTGCCTAATGGAACGCATGCCTATCTATCAGCGATGAA
TCTTTAGCAAATATCCCCATGCGGGGCCCCTGCTTCATGC
GGTATTAGTCCGACTTTCGCCGGGTTATCCCCCTCTGATA
GGTAAGTTGCATACGCGTTACTCACCCGTGCGCCGGTCGC
CACCCGGTATTGCTACCTGGTGCTGCCCCTCGACTGCAT
SEQ ID NO: 23
Strain ref. 15 (Parabacteroides sp.) 16S
ribosomal RNA gene, assembled using
Geneious:
GCGAGGTATCGAGACTACTAGGTACCCCCGGCTCCCATGG
CTTGACGGGCGGTGTGTACAAGGCCCGGGAACGTATTCAC
CGCGCCATGGCTGATGCGCGATTACTAGCGAATCCAGCTT
CACGGAGTCGGGTTGCAGACTCCGATCCGAACTGAGACGT
GGTTTGGGGATTCGCTCCCTGTCGCCAGGTGGCATCCCTT
TGTCCACGCCATTGTAACACGTGTGTCGCCCCGGATGTAA
GGGCCGTGCTGATTTGACGTCATCCCCGCCTTCCTCGCAG
CTTACGCTGGCAGTCCCACCAGAGTCCTCAGCTTTACCTG
TTAGTAACTAGTGGCAAGGGTTGCGCTCGTTATGGCACTT
AAGCCGACACCTCACGGCACGAGCTGACGACAACCATGCA
GCACCTCGCAAATCGCTATTGCTAGAGGCTCTGTTTCCAC
ATCGGTCCAAATGCGTTCAAACCCGGGTAAGGTTCCTCGC
GTATCATCGAATTAAACCACATGTTCCTCCGCTTGTGCGG
GCCCCCGTCAATTCCTTTGAGTTTCACCGTTGCCGGCGTA
CTCCCCAGGTGGATCACTTAACGCTTTCGCTGTGCCGCTT
ACCGTGTATCGCAAACAGCTAGTGATCATCGTTTACTGCG
TGGACTACCAGGGTATCTAATCCTGTTTGATCCCCACGCT
TTCGTGCATCAGCGTCAGTCATGGCTTGGCAGGCTGCCTT
CGCAATCGAGGTTCTGCGTGATATCTAAGCATTTCACCGC
TACACCACGCATTCCGCCTGCCTCAAACATACTCAAGCCC
CCCAGTTTCAACGGCAATTCTATGGTTGAGCCACAGACTT
TCACCGCTGACTTAAAAGGCCGCCTACGCACCCTTTAAAC
CCAATAAATCCGGATAACGCTCGGATCCTCCGTATTACCG
CGGCTGCTGGCACGGAGTTAGCCGATCCTTATTCATAAGG
TACATACAAAACRGGACACGTCCCGCACTTTATTCCCTTA
TAAAAGAGGTTTACGATCCATAGAACCTTCATCCCTCACG
CGACTTGGCTGGTTCAGGCTTACGCCCATTGACCAATATT
CCTCACTGCTGCCTCCCGTTGGAGTTTGGTCCGTGTCTCA
GTACCAATGTGGGGGACCTTCCTCTCAGAACCCCTATCCA
TCGTCGGTTTGGTGGGCCGTTACCCCGCCAACTGCATAAT
GGAACGCATGCCTATCTATCAGCGATGAATCTTTAGCAAA
TATCCCCATGCGGGACCCCTGCTTCATGCGGTATTAGTCC
GACTTTCGCCGGGTTATCCCCCTCTGATAGGTAAGTTGCA
TACGCGTTACTCACCCGTGCGCCGGTCGCCACCCGGTATT
GCTACGGGTGA

### REFERENCES

[1] Spor et al. (2011) Nat Rev Microbiol. 9(4):279-90.
[2] Eckburg et al. (2005) Science. 10; 308(5728):1635-8.
[3] Macpherson et al. (2001) Microbes Infect. 3(12):1021-35
[4] Macpherson et al. (2002) Cell Mol Life Sci. 59(12):2088-96.
[5] Mazmanian et al. (2005) Cell 15; 122(1):107-18.
[6] Frank et al. (2007) PNAS 104(34):13780-5.
[7] Scanlan et al. (2006) J Clin Microbiol. 44(11):3980-8.
[8] Kang et al. (2010) Inflamm Bowel Dis. 16(12):2034-42.
[9] Machiels et al. (2013) Gut. 63(8):1275-83.
[10] WO 2013/050792
[11] WO 03/046580
[12] WO 2013/008039
[13] WO 2014/167338
[14] Goldin and Gorbach (2008) Clin Infect Dis. 46 Suppl 2:S96-100.
[15] Azad et al. (2013) BMJ. 347:f6471.
[16] WO2016/203220
[17] Koh et al., International Journal of Cancer, volume 143, issue 7, pages 1797-1805
[18] Wu et al., Gut, doi: 10.1136/gutjnl-2017-315458
[19] Sakamoto and Benno (2006) Int J Syst Evol Microbiol. 56(Pt 7):1599-605.
[20] Masco et al. (2003) Systematic and Applied Microbiology, 26:557-563.
[21] Srůtková et al. (2011) J. Microbiol. Methods, 87(1):10-6.
[22] Seubert et al, JI, 2008, 180:5402-5412
[23] Mohan et al, 2018, Immunobiology, 223 (6-7): 477-485
[24] Glenn and Whartenby (2014) World J Stem Cells; 6(5): 526-539.
[25] Heng et al. (2004) Cardiovasc Res. 2004 Apr. 1; 62(1):34-42.
[26] Rashidi et al. (2018) Biology of Blood and Marrow Transplantation 24, Issue 6, Pages 1260-1263
[27] Fulop et al (2013) Crit Rev Oncog. 2013; 18(6):489-513.
[28] Bektas et al. (2017) J Leukoc Biol.;102(4):977-988.
[29] Fulop et al (2016) Rev Invest Clin.;68(2):84-91.
[30] Fulop et al. (2018) Front Immunol.; 8:1960.
[31] Kubica & Brewer (2012), Mayo Clin Proc 87(1): 991-1003
[32] Viguer et al. (2004), J Immunol 173: 1444-1453
[33] McCarter et al. (2007). Arm Surg Oncol. 14(10): 2854-60
[34] Miyamoto-Shinohara et al. (2008) J. Gen. Appl. Microbiol., 54, 9-24.
[35] Cryopreservation and Freeze-Drying Protocols, ed. by Day and McLellan, Humana Press.
[36] Leslie et al. (1995) Appl. Environ. Microbiol. 61, 3592-3597.
[37] Mitropoulou et al. (2013) J Nutr Metab. (2013) 716861.
[38] Kailasapathy et al. (2002) Curr Issues Intest Microbiol. 3(2):39-48.
[39] Handbook of Pharmaceutical Excipients, 2nd Edition, (1994), Edited by A Wade and PJ Weller
[40] Remington's Pharmaceutical Sciences, Mack Publishing Co. (A. R. Gennaro edit. 1985)
[41] Handbook of Microbiological Media, Fourth Edition (2010) Ronald Atlas, CRC Press.
[42] Maintaining Cultures for Biotechnology and Industry (1996) Jennie C. Hunter-Cevera, Academic Press
[43] Strobel (2009) Methods Mol Biol. 581:247-61.
[44] Gennaro (2000) Remington: The Science and Practice of Pharmacy. 20th edition, ISBN: 0683306472.
[45] Molecular Biology Techniques: An Intensive Laboratory Course, (Ream et al., eds., 1998, Academic Press).
[46] Methods In Enzymology (S. Colowick and N. Kaplan, eds., Academic Press, Inc.)
[47] Handbook of Experimental Immunology, Vols. I-IV (D. M. Weir and C. C. Blackwell, eds, 1986, Blackwell Scientific Publications)
[48] Sambrook et al. (2001) Molecular Cloning: A Laboratory Manual, 3rd edition (Cold Spring Harbor Laboratory Press).
[49] Handbook of Surface and Colloidal Chemistry (Birdi, K. S. ed., CRC Press, 1997)
[50] Ausubel et al. (eds) (2002) Short protocols in molecular biology, 5th edition (Current Protocols).
[51] PCR (Introduction to Biotechniques Series), 2nd ed. (Newton & Graham eds., 1997, Springer Verlag)
[52] Current Protocols in Molecular Biology (F. M. Ausubel et al., eds., 1987) Supplement 30
[53] Smith & Waterman (1981) Adv. Appl. Math. 2: 482-489
[54] Okemoto et al. (2006). J. Immunol. 176, 2: 1203-1208
[55] Su et al. (2008). Vaccine. 26, 40; 5111-22
[56] Bronte et al. (2013) Immunity. 2013 14; 39(5): 806-818
[57] Seubert et al, JI, 2008, 180:5402-5412
[58] Berraondo (2019) British Journal of Cancer 120:6-15

1.-27. (canceled)

28. A method of treating, preventing, or delaying the progression of a disease or a condition associated with immunosenescence in a subject in need thereof, the method comprising administering to the subject a therapeutically effective amount of a pharmaceutical composition comprising a bacterial strain of the genus Parabacteroides and a pharmaceutically acceptable excipient, diluent, or carrier, wherein the bacterial strain comprises a 16s rRNA gene sequence that has at least 95% sequence identity to SEQ ID NO: 9, 10, 11, 12, 13, 15, 16, 17, 18, or 19.

29. The method of claim 28, wherein the immunosenescence is characterized by a decrease in the number of B cells.

30. The method of claim 28, wherein the administering increases the number of B cells.

31. The method of claim 30, wherein the B cells comprise CD19+CD3−B cells.

32. The method of claim 28, wherein the disease or the condition associated with immunosenescence comprises a cardiovascular disease, a neurodegenerative disease, a cancer, type 2 diabetes, or an autoimmune disease.

33. The method of claim 32, wherein the neurodegenerative disease comprises Alzheimer's disease or Parkinson's disease.

34. The method of claim 28, wherein the composition is formulated for oral administration.

35. The method of claim 28, wherein the bacterial strain is lyophilized.

36. The method of claim 28, wherein the therapeutically effective amount comprises from about 1×10³ to about 1×10¹¹ colony forming units per gram (CFU/g) with respect to the weight of the pharmaceutical composition.

37. The method of claim 28, wherein the bacterial strain comprises a bacterial strain of the species Parabacteroides distasonis, Parabacteroides goldsteinii, or Parabacteroides merdae.

38. The method of claim 28, wherein the bacterial strain comprises a 16s rRNA gene sequence that has at least 97% sequence identity to SEQ ID NO: 9, 10, 11, 12, 13, 16, 17, 18, or 19.

39. The method of claim 28, wherein the bacterial strain comprises a 16s rRNA gene sequence that has at least 98% sequence identity to SEQ ID NO: 9, 10, 11, 12, 13, 15, 16, 17, 18, or 19.

40. The method of claim 28, wherein the bacterial strain comprises a 16s rRNA gene sequence represented by SEQ ID NO: 9, 10, 11, 12, 13, 15, 16, 17, 18, or 19.

41. The method of claim 28, wherein the bacterial strain is the bacterial strain deposited under accession number 42382 at NCIMB.

42. A method of enhancing an immune response against an antigen in a subject in need thereof, the method comprising administering to the subject a therapeutically effective amount of a pharmaceutical composition comprising a bacterial strain of the genus Parabacteroides and a pharmaceutically acceptable excipient, diluent, or carrier, wherein the bacterial strain comprises a 16s rRNA gene sequence that has at least 95% sequence identity to SEQ ID NO: 9, 10, 11, 12, 13, 15, 16, 17, 18, or 19.

43. The method of claim 42, wherein the administering increases the expression level and/or activity of one or more of MCP-1, IL-5, CXCL1, IP-10, RANTES, MIP-1α, MIP-1β, MIP-2, GM-CSF and TNFα.

44. The method of claim 42, wherein the antigen comprises a pathogen antigen, a neoantigen, a glycoprotein antigen, an archaea antigen, or a tumor antigen.

45. The method of claim 42, wherein the pathogen antigen comprises a viral antigen, a bacterial antigen, a fungal antigen, or a parasite antigen.

46. The method of claim 42, wherein the bacterial strain comprises a 16s rRNA gene sequence that has at least 97% sequence identity to SEQ ID NO: 9, 10, 11, 12, 13, 15, 16, 17, 18, or 19.

47. The method of claim 42, wherein the bacterial strain is the bacterial strain deposited under accession number 42382 at NCIMB.


SEQUENCE LISTING (23 sequences)

SEQ ID NO: 1 - DNA, 1488 bp, Parabacteroides distasonis
agagtttgatcctggctcaggatgaacgctagcgacaggcttaacacatgcaagtcgaggggcagcggggtgtagcaatacaccgccggcgaccggcgcacgggtgagtaacgcgtatgcaacttgcctatcagagggggataacccggcgaaagtcggactaataccgcatgaagcagggatcccgcatgggaatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtaagcctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggttaggctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactggtgactggggctaagtcgtaacaaggtaacc

SEQ ID NO: 2 - DNA, 1486 bp, Parabacteroides distasonis
agagtttgatcctggctcaggatgaacgctagcgacaggcttaacacatgcaagtcgaggggcagcacaggtagcaataccgggtggcgaccggcgcacgggtgagtaacgcgtatgcaacttacctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtaagcctgaaccagccaagtcgcgtgagggatgaaggttctatggatcgtaaacctcttttataagggaataaagtgcgggacgtgtcctgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcttagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacagtgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactggtgactggggctaagtcgtaacaaggtaacc

SEQ ID NO: 3 - DNA, 1486 bp, Parabacteroides distasonis
agagtttgatcctggctcaggatgaacgctagcgacaggcttaacacatgcaagtcgaggggcagcacaggtagcaatacccgccggcgaccggcgcacgggtgagtaacgcgtatgcaacttgcctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtaagcctgaaccagccaagtcgcgtgagggatgaaggttctatggatcgtaaacctcttttataagggaataaagtgtgggacgtgtcctgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactgggaggcttgagtatgtttgaggcaggcggaatgcgtggtgtagcggtgaaatgcttagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtgatgctgaggactctggtgggactgccagcgtaagctgcgaggaaggcggggatgacgtcaaatcagcacggcccttacatccggggcgacacacgtgttacaatggcgtggacaaagggatgccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactggtgactggggctaagtcgtaacaaggtaacc

SEQ ID NO: 4 - DNA, 1486 bp, Parabacteroides distasonis
agagtttgatcctggctcaggatgaacgctagcgacaggcttaacacatgcaagtcgaggggcagcacaggtagcaataccgggtggcgaccggcgcacgggtgagtaacgcgtatgcaacttacctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtaagcctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactgggaggcttgagtatgtttgaggcaggcggaatgcgtggtgtagcggtgaaatgcttagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactggtgactggggctaagtcgtaacaaggtaacc

SEQ ID NO: 5 - DNA, 1486 bp, Parabacteroides distasonis
agagtttgatcctggctcaggatgaacgctagcgacaggcttaacacatgcaagtcgaggggcagcacaggtagcaataccgggtggcgaccggcgcacgggtgagtaacgcgtatgcaacttacctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtaagcctgaaccagccaagtcgcgtgagggatgaaggttctatggatcgtaaacctcttttataagggaataaagtgtgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactgggaggcttgagtatgtttgaggcaggcggaatgcgtggtgtagcggtgaaatgcttagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacattgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaggccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgaaaggatcggcctagggtaaaactggtgactggggctaagtcgtaacaaggtaacc

SEQ ID NO: 6 - DNA, 1486 bp, Parabacteroides distasonis; identical to SEQ ID NO: 5

SEQ ID NO: 7 - DNA, 1492 bp, Parabacteroides merdae
agagtttgatcctggctcaggatgaacgctagcgacaggcttaacacatgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactccttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtaacc

SEQ ID NO: 8 - DNA, 1492 bp, Parabacteroides merdae
agagtttgatcctggctcaggatgaacgctagcgacaggcttaacacatgcaagtcgaggggcagcatgatttgtagcaatacagattgatggcgaccggcgcacgggtgagtaacgcgtatgcaacttacctatcagagggggatagcccggcgaaagtcggattaataccccataaaacaggggttccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactccttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtaacc

SEQ ID NO: 9 - DNA, 1403 bp, Parabacteroides distasonis
  Parabacteroides distasonis strain 755 or NCIMB 42382
amccgggtggcgaccggcgcacgggtgagtaacgcgtatgcaacttgcctatcagagggggataacccggcgaaagtcggactaataccgcatgaagcagggatcccgcatgggaatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtgagcctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactgggaggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggacmgakgtggaaacacattttctagcaatagccatttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgcgaggatcggcctagggtaaaactggtgactggggctaagtcgtacgggg

SEQ ID NO: 10 - DNA, 2002 bp, Parabacteroides goldsteinii
  Parabacteroides goldsteinii strain DSMZ 19448 or JCM13446
gtttgatcctggctcaggatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagacgccggcaacggtgaaactcaaaggaattgacgggggcccgcacaagcggaggaacatgtggtttaattcgatgatacgcgaggaaccttacccgggtttgaacgcatattgacagctctggaaacagagtctctagtaatagcaatttgcgaggtgctgcatggttgtcgtcagctcgtgccgtgaggtgtcggcttaagtgccataacgagcgcaacccttatcactagttactaacaggtcatgctgaggactctagtgagactgccagcgtaagctgtgaggaaggtggggatgacgtcaaatcagcacggcccttacatccggggcgacacacgtgttacaatggtggggacaaagggcagctaccgtgtgagcggatgcgaatctccaaaccccatctcagttcggatcgaagtctgcaacccgacttcgtgaagctggattcgctagtaatcgcgcatcagccatggcgcggtgaatacgttcccgggccttgtacacaccacccgtcaagccatgggagttgggggtacctaaagtccgtaaccgcaaggatcggcctagggtaaaaccgatgactggggctaagtcgtaacaaggtagccgtaccggaaggtgcggctggaacacctcctttctggagcgcagagttcgttatcaagttgactcagaggtattagttaacttgtactacggttgaatatgtataaaatatagatctaccggcaataaagtgtcggcaagagagaaaaatgatgctgagggaaaccaaggcaaagttgacagtcctatagctcagttggttagagcgctacactgataatgtagaggtcggcagttcaactctgcctgggactacagaatctctaagagagaattttgggggattagctcagctggctagagcatctgccttgcacgcagagggtcaacggttcgaatccgttattctccacaaaaagttaccgagacatcagaaacgtaaagtaacgacaagatctttgacatgatggacaacgtaaaataaagtaacaagagcaagctgaagatatatcaatccgatttacccctgtggtaaccggaaataagaaagtaagcaagggcagacggtggatgccttggc

SEQ ID NO: 11 - DNA, 1428 bp, Parabacteroides goldsteinii
  Parabacteroides goldsteinii strain DSMZ29187 or BS-C3-2 16S
ctggctcaggatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaaacgtgttcctttttgtatgtaccatatgaataagcatcggctaacttccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgcattcggaccggagtggaaacacttcttctagtaatagccgtttgcgaggtgctgcatggttgtcgtcagctcgtgccgtgaggtgtcggcttaagtgccataacgagcgcaacccttatcactagttactaacaggtcaagctgaggactctagtgagactgccagcgtaagctgtgaggaaggtggggatgacgtcaaatcagcacggcccttacatccggggcgacacacgtgttacaatggtggggacaaagggcagctacctggcgacaggatgctaatctccaaacctcatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaagg

SEQ ID NO: 12 - DNA, 1397 bp, Parabacteroides distasonis
aaggccgatccttgtcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcaccaggtggcctccctttgtccacgccattgtaacacgtgtgtcgccccggatgtaagggccgtgctgatttgacgtcatccccgccttcctcgcagcttacgctggcagtcccaccagagtcctcagctttacctgttagtaactagtggcaagggttgcgctcgttatggcacttaagccgacacctcacggcacgagctgacgacaaccatgcagcacctcgcaaacggctattgctagaaaaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtcatggcttggcaggctgccttcgcaatcggggttctgcgtgatatctatgcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcagcctctcggccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttagcaaatatccccatgcggggcccctgcttcatgcggtattagtccgactttcgccgggttatccccctctgataggtaagttgcatacgcgttactcacccgtgcgccggtcgccacccggtattgctacctgtgctgccgcctcgactgca

SEQ ID NO: 13 - DNA, 1403 bp, Parabacteroides distasonis
aggccgatccttgtcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcatccctttgtccacgccattgtaacacgtgtgtcgccccggatgtaagggccgtgctgatttgacgtcatccccgccttcctcgcagcttacgctggcagtcccaccagagtcctcagctttacctgttagtaactagtggcaagggttgcgctcgttatggcacttaagccgacacctcacggcacgagctgacgacaaccatgcagcacctcgcaaatcgctattgctagaggctgtgtttccacagcggtccaaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttaccgtgtatcgcaaacagctagtgatcatcgtttactgcgtggactaccagggtatctaatcctgtttgatccccacgctttcgtgcatcagcgtcagtcatggcttggcaggctgccttcgcaatcggggttstgcgtgatatctaagcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcaggcttacgcccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttagcaaatatccccatgcgggacccctgcttcatgcggtattagtccgactttcgccgggttatccccctctgataggtaagttgcatacgcgttactcacccgtgcgccggtcgccacccggtattgctacctgtgctgcccctcgacttgcatgtgtaa

SEQ ID NO: 14 - DNA, 1430 bp, Parabacteroides sp.
gggcccaatttaactaggccgatccttgcggttacggacttcaggtacccccggctcccatggcttgacgggcggtgtgtacaaggcccgggaacgtattcaccgcgccatggctgatgcgcgattactagcgaatccagcttcacggagtcgagttgcagactccgatccgaactgagacatggtttggagatttgcatcacatcgctgtgtagctgccctttgtccatgccattgtaacacgtgtgtcgccccggatgtaagggccgtgctgatttgacgtcatccccaccttcctcacagcttacgctggcagtctcaccagagtcctcagcttgacctgttagtaactagtgataagggttgcgctcgttatggcacttaagccgacacctcacggcacgagctgacgacaaccatgcagcacytcgcaaacggtcattgctgaaaggagcgtttccactccggtccgaatgcgttcaaacccgggtaaggttcctcgcgtatcatcgaattaaaccacatgttcctccgcttgtgcgggcccccgtcaattcctttgagtttcaccgttgccggcgtactccccaggtggattacttaacgctttcgctgtagagcttactgtctatcgcaaactcctagtaatcatcgtttactgcgtggactaccagggtatctaatcctgtttgatccccacgctttcgtgcttcagtgtcagttatagtttagtaagctgccttcgcaatcggagttctgcgtgatatctatgcatttcaccgctacaccacgcattccgcctacctcaaatatactcaagtcatccagtttcaacggcaattttatggttgagccacaaactttcaccgctgacttaaacaaccacctacgcaccctttaaacccaataaatccggataacgctcgcatcctccgtattaccgcggctgctggcacggagttagccgatgcttattcatacggtacatacaaaatgggacacgtcccacactttattcccskataaaagaagtttacaaaccatagatccttcatccttcacgcgacttggctggttcagcctcccggccattgaccaatattcctcactgctgcctcccgtaggartttggaccgtgtctcagttccaatgtgggggaccttcctctcagaacccctatccatcgtcggtttggtgggccgttaccccgccaactgcctaatggaacgcatgcctatctatcagcgatgaatctttaacaaatattcccatgcgggacccctgttttatggagcattaatccgactttcgccgggctattcccctctgataggcaagttgcatacgcgttactcacccgtgcgccggtcgccggcaggcattgctgcccccgctgcccctcgacttgcatggttagcctccaattcccc

SEQ ID NO: 15 - DNA, 1402 bp, Parabacteroides distasonis
taggccgatccttgcggttacggacttcaggtacccccggctcccatggcttgacgggcggtgtgtacaaggcccgggaacgtattcaccgcgccatggctgatgcgcgattactagcgaatccagcttcacggagtcgagttgcagactccgatccgaactgagacatggtttggagatttgcatcacatcgctgtgtagctgccctttgtccatgccattgtaacacgtgtgtcgccccggatgtaagggccgtgctgatttgacgtcatccccaccttcctcacagcttacgctggcagtctcaccagagtcctcagcttgacctgttagtaactagtgataagggttgcgctcgttatggcacttaagccgacacctcacggcacgagctgacgacaaccatgcagcacctcgcaaacggtcattgctgaaaggagcgtttccactccggtccgaatgcgttcaaacccgggtaaggttcctcgcgtatcatcgaattaaaccacatgttcctccgcttgtgcgggccccygtcaattcctttgagtttcaccgttgccggcgtactccccaggtggattacttaacgctttcgctgtagagcttactgtctatcgcamactcctagtaatcatcgtttactgcgtggactaccagggtatctaatcctgtttgatccccacgctttcgtgcttcagtgtcagttatagtttagtaagctgccttcgcaatcggagttctgcgtgatatctatgcatttcaccgctacaccacgcattccgcctacctcaaatatactcaagtcatccagtttcaacggcaattttatggttgagccacaaactttcaccgctgacttaaacaaccacctacgcaccctttaaacccaataaatccggataacgctcgcatcctccgtattaccgcggctgctggcacggagttagccgatgcttattcatacggtacatacaaaatgggacacgtcccacactttattcccgtataaaagaagtttacaaaccatagatccttcatccttcacgcgacttggctggttcagcctcccggccattgaccaatattcctcactgctgcctcccgtaggagtttggaccgtgtctcagttccaatgtgggggaccttcctctcagaacccctatccatcgtcggtttggtgggccgttaccccgccaactgcctaatggaacgcatgcctatctatcagcgatgaatctttaacaaatattcccatgcgggacccctgttttatggagcattaatccgactttcgccgggctattcccctctgataggcaagttgcatacgcgttactcacccgtgcgccggtcgccggcaggcattgctgcccccgctgcccctcgacttgcatgtgtt

SEQ ID NO: 16 - DNA, 1405 bp, Parabacteroides distasonis
gtaggccgatcctcgcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcttccctttgtccacgccattgtaacacgtgtgtcgccccggatgtaagggccgtgctgatttgacgtcatccccgccttcctcgcagcttacgctggcagtcccaccagagtcctcagcytwacctgttagtaactagtggcaagggttgcgctcgttatggcacttaagccgacacctcacggcacgagctgacgacaaccatgcagcacctcgcaaacggctattgctagaaaaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacactgtatcgcaaacagctagtgatcatcgtttactgcgtggactaccagggtatctaatcctgtttgatccccacgctttcgtgcatcagcgtcagtcatggcttggcaggctgccttcgcaatcggggttctgcgtgatatctatgcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcctacactttattcccttataaaagaggtttacgatccatagaaccttcatccctcacgcgacttggctggttcaggcttacgcccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcmtatmtatcagcgatgwatcttkmgcaaatatccccrtgcggggcccgtgcttcrtgcggtattagtcmgactttcgccgggttatccccctctgataggyaagttgcatacgcgttactcacccgtgcgccggtcgccrgccgcggtatctgctaccccgcgctgcccctcgacttgcatggt

SEQ ID NO: 17 - DNA, 1365 bp, Parabacteroides distasonis
gatcctcgcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcctccctttgtccacgccattgtaacacgtgtgtcgccccggatgtaagggccgtgctgatttgacgtcatccccgccttcctcgcagcttacgctggcagtcccaccagagtcctcagcatcacctgttagtaactagtggcaagggttgcgctcgttatggcacttaagccgacacctcacggcacgagctgacgacaaccatgcagcacctcgcaaacggctattgctagaaaaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtcatggcttggcaggctgccttcgcaatcggggttctgcgtgatatctatgcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcagcctttcggccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttagcaaatatccccatgcggggcccctgcttcatgcggtattagtccgactttcgccgggttatccccctctgataggtaagttgcatacgcgttactcacccgtgcgccggtcgccacccggtattgc

SEQ ID NO: 18 - DNA, 1415 bp, Parabacteroides merdae
ttaaataggccgatccttgcggttacggacttcaggtacccccggctcccatggcttgacgggcggtgtgtacaaggcccgggaacgtattcaccgcgccatggctgatgcgcgattactagcgaatccagcttcacggagtcgagttgcagactccgatccgaactgagacatggtttggagattagcatcctgtcaccaggtagctgccctttgtccatgccattgtaacacgtgtgtcgccccggatgtaagggccgtgctgatttgacgtcatccccaccttcctcacagcttacgctggcagtctcaccagagtcctcagcttcacctgttagtaactagtgataagggttgcgctcgttatggcacttaagccgacacctcacggcacgagctgacgacaaccatgcagcacctcgtaatctgctattgctagaaagagtgtttccactccggtcagactacgttcaaacccgggtaaggttcctcgcgtatcatcgaattaaaccacatgttcctccgcttgtgcgggcccccgtcaattcctttgagtttcaccgttgccggcgtactccccaggtggattacttaacgctttcgctgtagagcttacattgtatcgcaaactcctagtaatcatcgtttactgcgtggactaccagggtatctaatcctgtttgatccccacgctttcgtgcttcagtgtcagttatggtttagtaagctgccttcgcaatcggagttctgcgtgatatctatgcatttcaccgctacaccacgcattccgcctacctcaaacacactcaagtaacccagtttcaacggcaattttatggttgagccacaaactttcaccgctgacttaaatcaccacctacgcaccctttaaacccaataaatccggataacgctcgcatcctccgtattaccgcggctgctggcacggagttagccgatgcttattcatagggtacatacaaaaaaggacacgtcctccactttattcccctataaaagaagtttacaaaccatagatccttcttccttcacgcgacttggctggttcagcctctcggccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttaacaaatattcccatgcgggacccctgttttatggggtattaatccgactttcgccgggctatccccctctgataggtaagttgcatacgcgttactcacccgtgcgccggtcgccatcaatctgtattgctacaaatcatgctgcccctcgacttgcatggttaag

SEQ ID NO: 19 - DNA, 1390 bp, Parabacteroides distasonis
gatctcgcggtttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcctccctttgtccacgccattgtaacacgtgtgtcgccccggatgtaagggccgtgctgatttgacgtcatccccgccttcctcgcagcttacgctggcagtcccaccagagtcctcagcatcacctgttagtaactagtggcaagggttgcgctcgttatggcacttaagccgacacctcacggcacgagctgacgacaaccatgcagcacctcgcaaacggctattgctagaaaaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtcatggcttggcaggctgccttcgcaatcggggttctgcgtgatatctatgcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcagcctttcggccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttagcaaatatccccatgcggggcccctgcttcatgcggtattagtccgactttcgccgggttatccccctctgataggtaagttgcatacgcgttactcacccgtgcgccggtcgccacccggtattgctacctggtgctgccccctcgactgc

SEQ ID NO: 20 - DNA, 1399 bp, Parabacteroides sp.
ccgatcctttcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcctccctttgtccacgccattgtaacacgtgtgtcgccccggatgtaagggccgtgctgatttgacgtcatccccgccttcctcgcagcttacgctggcagtcccaccagagtcctcagcatcacctgttagtaactagtggcaagggttgcgctcgttatggcacttaagccgacacctcacggcacgagctgacgacaaccatgcagcacctcgcaaatcgctatcgctagagactctgtttccagagctgtcgaaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtaatggcttggcaggctgccttcgcaatcggggttctgcgtgatatctatgcatttcaccgctacaccacgcattcygcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacmaaacgggacacgtcccgcactttattcccttataaaagaggtttacgatccatagaaccttcatccctcacgcgacttggctggttcagsctctcgcccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcmwgcckatytatcagcgawgaatctttagcaaatatccccatgcggggcccctgcttcmtgcggtattagtccgactttcgccgggttatccccctctgataggcaagtwgcatacgcgttactcacccgtgcgccggtcgccacccggtattgctaccctgygctgcccctcgacttgcatgktaa

SEQ ID NO: 21 - DNA, 1431 bp, Parabacteroides sp.
  n 539: c, g, t or a
  n 827: c, g, t or a
gcgcggtttaactaggccgatcctttcggttacggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcctccctttgtccacgccattgtaacacgtgtgtcgccccggatgtaagggccgtgctgatttgacgtcatccccgccttcctcgcagcttacgctggcagtcccaccagagtcctcagcatcacctgttagtaactagtggcaagggttgcgctcgttatggcacttaagccgacacctcacggcacgagctgacgacaaccatgcagcacctcgcaaatcgctatcgctagagactctgtttccagagctgtcgaaatgcgttcaaacccgggtaaggttcctcgcgtatcatcgaattaaaccacatgttcctccgcttgtgncgggcccccgtcaattcctttgagtttcaccgttgccggcgtaytccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtaatggcttggcaggctgccttcgcaatcggggttctgcgtgatatctatgcatttcaccgctacaccacgcattccgcctgcctcaaacatactcaagccccccantttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcagcctttcggccattgaccaatattcctcactgctgcctcccgtaggagtttggtccgtgtctcagtaccaatgtgggggaccttcctctcagaacccctatccattgtcggtttggtgggccgttaccccgccaactgcctaatggaacgcatgcctatctatcagcgatgaatctttagcaaatatccccatgcggggcccctgcttcatgcggtattagtccgactttcgccgggttatccccctctgataggcaagttgcatacgcgttactcacccgtgcgccggtcgccgagccgcggtattgctaccctcgtgctgcccctcgacttgcatggttagcctccatccc

SEQ ID NO: 22 - DNA, 1399 bp, Parabacteroides sp.
cttaggccgatccctcgcggttcggacttcaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcctccctttgtccacgccattgtaacacgtgtgtcgccccggatgtaagggccgtgctgatttgacgtcatccccgccttcctcgcagcttacgctggcagtcccaccagagtcctcagcatcacctgttagtaactagtggcaagggttgcgctcgttatggcacttaagccgacacctcacggcacgagctgacgacaaccatgcagcacctcgcaaacggctattgctagaaaaggtgtttccacctcggtccgaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttacagtgtatcgcaaacagctagtgatcatcgtttactgcgtggactaccagggtatctaatcctgtttgatccccacgctttcgtgcatcagcgtcagtcatggcttggcaggctgccttcgcaatcggggttctgcgtgatatctatgcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcagcctttcggccattgaccaatattcctcactgctgcctcccgtaggagtttggtccgtgtctcagtaccaatgtgggggaccttcctctcagaacccctatccatcgtcggtttggtgggccgttaccccgccaactgcctaatggaacgcatgcctatctatcagcgatgaatctttagcaaatatccccatgcggggcccctgcttcatgcggtattagtccgactttcgccgggttatccccctctgataggtaagttgcatacgcgttactcacccgtgcgccggtcgccacccggtattgctacctggtgctgcccctcgactgcat

SEQ ID NO: 23 - DNA, 1371 bp, Parabacteroides sp.
gcgaggtatcgagactactaggtacccccggctcccatggcttgacgggcggtgtgtacaaggcccgggaacgtattcaccgcgccatggctgatgcgcgattactagcgaatccagcttcacggagtcgggttgcagactccgatccgaactgagacgtggtttggggattcgctccctgtcgccaggtggcatccctttgtccacgccattgtaacacgtgtgtcgccccggatgtaagggccgtgctgatttgacgtcatccccgccttcctcgcagcttacgctggcagtcccaccagagtcctcagctttacctgttagtaactagtggcaagggttgcgctcgttatggcacttaagccgacacctcacggcacgagctgacgacaaccatgcagcacctcgcaaatcgctattgctagaggctctgtttccacatcggtccaaatgcgttcaaacccgggtaaggttcctcgcgtatcatcgaattaaaccacatgttcctccgcttgtgcgggcccccgtcaattcctttgagtttcaccgttgccggcgtactccccaggtggatcacttaacgctttcgctgtgccgcttaccgtgtatcgcaaacagctagtgatcatcgtttactgcgtggactaccagggtatctaatcctgtttgatccccacgctttcgtgcatcagcgtcagtcatggcttggcaggctgccttcgcaatcgaggttctgcgtgatatctaagcatttcaccgctacaccacgcattccgcctgcctcaaacatactcaagccccccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacrggacacgtcccgcactttattcccttataaaagaggtttacgatccatagaaccttcatccctcacgcgacttggctggttcaggcttacgcccattgaccaatattcctcactgctgcctcccgttggagtttggtccgtgtctcagtaccaatgtgggggaccttcctctcagaacccctatccatcgtcggtttggtgggccgttaccccgccaactgcataatggaacgcatgcctatctatcagcgatgaatctttagcaaatatccccatgcgggacccctgcttcatgcggtattagtccgactttcgccgggttatccccctctgataggtaagttgcatacgcgttactcacccgtgcgccggtcgccacccggtattgctacgggtga